(19) United States
(12) Patent Application Publication
(10) Pub. No.: US 2025/0205326 A1
(43) Pub. Date: Jun. 26, 2025

(54) COMPOSITIONS OF VACCINES AGAINST CLOSTRIDIAL DERMATITIS AND METHODS OF USE THEREOF

(71) Applicant: NORTH CAROLINA STATE UNIVERSITY, Raleigh, NC (US)

(72) Inventors: Raveendra Rangarao Kulkarni, Raleigh, NC (US); Feba Ann John, Raleigh, NC (US)

(21) Appl. No.: 18/941,161

(22) Filed: Nov. 8, 2024

Related U.S. Application Data
(60) Provisional application No. 63/597,112, filed on Nov. 8, 2023.
(60) Provisional application No. 63/608,491, filed on Dec. 11, 2023.
(60) Provisional application No. 63/609,022, filed on Dec. 12, 2023.

(51) Int. Cl. A61K 39/08; A61K 39/00; A61P 37/04

(52) U.S. Cl. CPC A61K 39/08; A61P 37/04; A61K 2039/523; A61K 2039/552

(57) ABSTRACT

The present disclosure provides vaccine compositions, kits, and/or vectors for preventing, decreasing, reducing, and/or ameliorating spread of bacterial pathogens causing Clostridial dermatitis/Gangrenous dermatitis/Cellulitis in poultry animals, and methods of use thereof.

### CROSS REFERENCE TO RELATED APPLICATIONS

This application claims priority to, and the benefit of, U.S. Provisional Patent Application No. 63/597,112, filed Nov. 8, 2023, No. 63/608,491, filed Dec. 11, 2023, and No. 63/609,022, filed Dec. 12, 2023, which is incorporated by reference herein in its entirety.

### REFERENCE TO SEQUENCE LISTING

The sequence listing submitted on Nov. 8, 2024, as an.XML file entitled 10620-114US1_ST26.xml created on Oct. 24, 2024, and having a file size of 36,513 bytes is hereby incorporated by reference pursuant to 37 C.F.R. § 1.52(e)(5).

### FIELD

The present disclosure relates to composition of vaccines against clostridial dermatitis and methods of use thereof.

### BACKGROUND

Clostridial dermatitis (CD), also referred to as necrotic dermatitis, gangrenous cellulitis, gangrenous dermatomyositis, gas edema disease, and blue wing disease, occurs in poultry. It has been an economically challenging disease with its prevalence in the United States being on the rise in the last 20 years. During the past two decades, the poultry industry, including the turkey industry, has experienced an increase in the frequency and severity of CD. CD affects about 40-50% of U.S. turkey grower farms, causing losses due to poor production rate, sudden spikes in mortality, therapeutic costs, and carcass condemnations. Despite its serious impact on turkey health and on the economy, no effective vaccines are currently available.

Given limitations of effective preventative measures to combat CD in poultry, there is need to address the aforementioned problems mentioned above by developing therapeutic and/or prophylactic compositions to prevent infections and spread of CD in the poultry industry.

### SUMMARY

Disclosed herein are vaccine composition, kits and methods of use thereof in treating clostridial dermatitis.

Accordingly, in one aspect, a vaccine composition comprising a non-toxic domain of an alpha toxin (ATX) protein and a pharmaceutically acceptable carrier, wherein the non-toxic domain comprises ntATX-D1, ntATX-D2, or a combination thereof. In some embodiments, the ntATX-D1 comprises at least 80% sequence identity to SEQ ID NO: 2. In some embodiments, the ntATX-D1 comprises SEQ ID NO: 2. In some embodiments, the ntATX-D2 comprises at least 80% sequence identity to SEQ ID NO: 4. In some embodiments, the ntATX-D2 comprises SEQ ID NO: 4. In some embodiments, the vaccine composition comprises at least one immunogenic fragment of SEQ ID NO: 2 and/or SEQ ID NO: 4. In some embodiments, the vaccine composition further comprising an additional toxin, or fragments thereof. In some embodiments, the vaccine composition further comprising an adjuvant. In some embodiments, the pharmaceutically acceptable carrier comprises an oil-in-water emulsion, a nano-emulsion, a nanoparticle, an excipient, a diluent, a salt, a buffer, a stabilizer, a lipid, or combinations thereof.

In one aspect, disclosed herein is a vector comprising a nucleic acid sequence encoding a non-toxic domain of an alpha toxin (ATX) protein, wherein the non-toxic domain comprises ntATX-D1, ntATX-D2, or a combination thereof. In some embodiments, ntATX-D1, ntATX-D2, or a combination thereof are expressed on the same vector or different vectors. In some embodiments, the nucleic acid sequence encoding ntATX-D1 comprises at least 80% sequence identity to SEQ ID NO: 1. In some embodiments, the nucleic acid sequence encoding ntATX-D1 comprises SEQ ID NO: 1. In some embodiments, the nucleic acid sequence encoding ntATX-D2 comprises at least 80% sequence identity to SEQ ID NO: 3. In some embodiments, the nucleic acid sequence encoding ntATX-D2 comprises SEQ ID NO: 3. In some embodiments, the nucleic acid sequence encodes an immunogenic fragment of ntATX-D1 or ntATX-D2. In some embodiments, the vector comprises a Nisin Controlled gene Expression (NICE) system. In some embodiments, the Nisin Controlled gene Expression (NICE) system comprises pNZ8124. In some embodiments, the vector serves as a vaccine.

In one aspect disclosed herein is a method of preventing clostridial dermatitis (CD) in a subject, the method comprises administering to the subject a pharmaceutically effective amount of a vaccine composition, wherein the vaccine composition comprises a non-toxic domain of an alpha toxin (ATX) protein and a pharmaceutically acceptable carrier, wherein the non-toxic domain comprises ntATX-D1, ntATX-D2, or a combination thereof.

In one aspects, disclosed herein is a method of preventing clostridial dermatitis (CD) in a subject, the method comprises administering a pharmaceutically effective amount of a vaccine composition, wherein the vaccine composition comprises a non-toxic domain of an alpha toxin (ATX) protein and a pharmaceutically acceptable carrier, wherein the non-toxic domain comprises ntATX-D1, ntATX-D2, or a combination thereof. In some embodiments, the vaccine composition comprises a vector, wherein the vector comprises a nucleic acid sequence encoding a non-toxic domain of an alpha toxin (ATX) protein, wherein the non-toxic domain comprises ntATX-D1, ntATX-D2, or a combination thereof. In some embodiments, CD is caused by a Clostridium bacterium.

In some embodiments, the vaccine composition is administered subcutaneously. In some embodiments, the vaccine composition is administered orally. In some embodiments, the vector comprises a Nisin Controlled gene Expression (NICE) system. In some embodiments, the Nisin Controlled gene Expression (NICE) system comprises pNZ8124. In some embodiments, the NICE system is derived from a food-grade Lactococcus lactis. In some embodiments, the food-grade Lactococcus lactis comprises expression strain, L. lactis-NZ9000. In some embodiments, the L. lactis-NZ9000 is at a concentration of about 1×10⁹ CFU/mL.

In some embodiments, the vaccine composition comprises at least two doses. In some embodiments, the vaccine composition comprises at least three doses comprising a first dose, second dose and a third dose. In some embodiments, the first dose comprises 3 ml of the vaccine composition, when the L. lactis-NZ9000 is at a concentration at least 1×10⁹ CFU/mL. In some embodiments, the second dose comprises 4 ml of the vaccine composition, when the L. lactis-NZ9000 is at a concentration at least 1×10⁹ CFU/mL. In some embodiments, the third dose comprises 5 ml of the vaccine composition, when the L. lactis-NZ9000 is at a concentration of 1×10⁹ CFU/mL. In some embodiments, the vaccine composition comprises G+M17 media.

In some embodiments, the vaccine composition produces at least a 50% decrease in gene expression level of at least one of TLR21, IL-1B, IL-6, and IFNγ genes in a tissue sample from an immunized subject compared to an unimmunized subject. In some embodiments, the tissue sample comprises skin, muscle, spleen or cecal tonsil (CT). In some embodiments, the vaccine composition produces at least a 20% increase in frequency of splenic CD4+ T cells, circulatory γδ T cells, B cells or a combination thereof in whole sera of an immunized subject compared to an unimmunized subject. In some embodiments, the method produces at least a 10% increase in body weight of an immunized subject exposed to a Clostridium bacterium compared to an unimmunized subject exposed to a Clostridium bacterium. In some embodiments, the method produces at least a 50% decrease in disease severity of an immunized subject exposed to a Clostridium bacterium compared to an unimmunized subject exposed to a Clostridium bacterium. In some embodiments, the vaccine composition produces at least a 40% decrease in mortality and/or at least 20% decrease in gross pathology of an immunized subject exposed to a Clostridium bacterium compared to an unimmunized subject exposed to a Clostridium bacterium. In some embodiments, the subject is a poultry animal. In some embodiments, the poultry animal is chicken or turkey.

Also disclosed herein is a kit comprising the vaccine composition of any of the preceding aspects.

### BRIEF DESCRIPTION OF FIGURES

The accompanying figures, which are incorporated in and constitute a part of this specification, illustrate several aspects described below.

FIGS. 1A-1E show the Construction and cloning of non-toxic domains of Clostridium septicum alpha toxin. Based on the C. septicum alpha toxin (referred to hereafter as ‘ATX’) sequence information, two non-toxic (nt) regions devoid of critical sites for toxicity were identified; the ntATX-D1 (912 bp) devoid of proteolytic cleavage site (Arg-367-Ser-368) and ntATX-D2 (531 bp) devoid of a signal peptide sequence (residue 1-31) and the region corresponding to cytolytic, pore-forming domain of ATX (residue 203-232), as shown in FIG. 1A. Genes encoding ntATX-D1 and ntATX-D2 were amplified from C. septicum genomic DNA (FIG. 1B) and were cloned first into the TA cloning vector (pCR2.1) and transformed into TOP10 competent E. coli cells followed by cloning into pET14b expression vector between the XhoI and BamHI sites, followed by transformation of plasmids into the E. coli DH5α (shuttle host). The histidine-tagged recombinant ntATX-D1 (˜45 kDa) or ntATX-D2 (˜25 kDa) proteins were expressed and purified from E. coli BL21-DE3 (expression host) as recombinant proteins and immunoreacted in Western blot assays with anti-His tag antibodies (FIG. 1C) as well as with the serum collected from turkeys experimentally infected with C. septicum (FIG. 1D). NCx-negative control; D1-ntATX-D1; D2-ntATX-D2; M-Marker (protein ladder). FIG. 1E shows the evaluation of toxicity (hemolytic activity), is any, associated with the purified ntATX-D1 and ntATX-D2 along with a positive control, using blood agar plates.

FIGS. 2A-2B shows the mortality and gross pathology lesions in turkeys following C. septicum challenge. Turkeys were immunized with subunit recombinant proteins at 7, 8 and 9 weeks of age followed by a C. septicum challenge at two weeks post-last immunization. Mortality was monitored for a period of 72 hours, and the gross lesions were scored, as described in the materials and methods section. NCx-negative control, unchallenged (n=10); PCx-positive control, unimmunized and challenged (n=13); D1-immunized with ntATX-D1 protein and challenged (n=15); D2-immunized with ntATX-D2 protein and challenged (n=15). Bars represent median score with range and the asterisks above the bars indicate statistical significance; *P<0.05, **P<0.01, or ***P<0.001.

FIGS. 3A-3C show the histopathological changes in immunized turkeys following C. septicum challenge. FIG. 3A represents changes in the skin tissue indicating lesion scores evaluated based on the criteria of inflammation (including fibrin/edema, heterophils, and lymphocytes/histocyte lesions), gangrenous dermatitis (including lesions of fibrin/edema, cell lysis, and bacteria) and caseous necrosis/granuloma lesions. FIG. 3B represents changes in the muscle tissue indicating lesion scores evaluated based on the criteria of inflammation (including fibrin/edema, heterophils, and lymphocytes/histocyte lesions), gangrenous dermatitis (including lesion scores for fibrin/edema, cell lysis, and bacteria, caseous necrosis and granuloma lesions, and muscle myopathy lesions. FIG. 3C represents changes in the spleen tissue indicating lesion scores evaluated based on the criteria of splenic necrosis and cell lysis, and splenic lymphoid depletion and hyperplasia. NCx-negative control, unchallenged (n=6); PCx-positive control, unimmunized and challenged (n=11); D1-immunized with ntATX-D1 protein and challenged (n=8); D2-immunized with ntATX-D2 protein and challenged (n=10). Bars represent median score with range and the asterisks above the bars indicate statistical significance; *P<0.05, **P<0.01 or ***P<0.001.

FIGS. 4A-4G shows the immune gene expression in skin tissue of immunized turkeys. Turkeys were immunized with subunit recombinant proteins at 7, 8 and 9 weeks of age followed by a C. septicum challenge at two weeks post-last immunization. Tissue samples from birds (n=8) in the experimental groups were collected at termination for RNA extraction and cDNA synthesis. Real-time PCR to quantify the expression of genes indicated in the figure was performed and the expression levels are shown as relative to β-actin reference gene. Negative Ctrl—Control, unchallenged; Positive Ctrl—Control, unimmunized and challenged; D1-immunized with ntATX-D1 protein and challenged; D2-immunized with ntATX-D2 protein and challenged. Graphs show standard error of mean bars with the asterisks above the bars indicating statistical significance; *P<0.05, **P<0.01 or ***P<0.001.

FIGS. 5A-5H shows the immune gene expression in muscle tissue of immunized turkeys. Turkeys were immunized with subunit recombinant proteins at 7, 8 and 9 weeks of age followed by a C. septicum challenge at two weeks post-last immunization. Tissue samples from birds (n=8) in the experimental groups were collected at termination for RNA extraction and cDNA synthesis. Real-time PCR to quantify the expression of genes indicated in the figure was performed and the expression levels are shown as relative to B-actin reference gene. Negative Ctrl-Control, unchallenged; Positive Ctrl-Control, unimmunized and challenged; D1-immunized with ntATX-D1 protein and challenged; D2-immunized with ntATX-D2 protein and challenged. Graphs show standard error of mean bars with the asterisks above the bars indicating statistical significance; *P<0.05, or ***P<0.001.

FIGS. 6A-6F shows the immune gene expression in cecal tonsil tissue of immunized turkeys. Turkeys were immunized with subunit recombinant proteins at 7, 8 and 9 weeks of age followed by a C. septicum challenge at two weeks post-last immunization. Tissue samples from birds (n=8) in the experimental groups were collected at termination for RNA extraction and cDNA synthesis. Real-time PCR to quantify the expression of genes indicated in the figure was performed and the expression levels are shown as relative to β-actin reference gene. CT-Cecal tonsil; Negative Ctrl-Control, unchallenged; Positive Ctrl-Control, unimmunized and challenged; D1-immunized with ntATX-D1 protein and challenged; D2-immunized with ntATX-D2 protein and challenged. Graphs show standard error of mean bars with the asterisks above the bars indicating statistical significance; *P<0.05 or **P<0.01.

FIGS. 7A-7G shows the immune gene expression in spleen tissue of immunized turkeys. Turkeys were immunized with subunit recombinant proteins at 7, 8 and 9 weeks of age followed by a C. septicum challenge at two weeks post-last immunization. Tissue samples from birds (n=8) in the experimental groups were collected at termination for RNA extraction and cDNA synthesis. Real-time PCR to quantify the expression of genes indicated in the figure was performed and the expression levels are shown as relative to β-actin reference gene. Negative Ctrl-Control, unchallenged; Positive Ctrl-Control, unimmunized and challenged; D1-immunized with ntATX-D1 protein and challenged; D2-immunized with ntATX-D2 protein and challenged. Graphs show standard error of mean bars with the asterisks above the bars indicating statistical significance; P<0.05 or **P<0.01.

FIGS. 8A-8G show the peripheral blood cellular response in immunized turkeys. Turkeys were immunized with subunit recombinant proteins at 7, 8 and 9 weeks of age followed by a C. septicum challenge at two weeks post-last immunization. Peripheral blood samples from birds (n=8) in the experimental groups were collected at termination for the preparation of single cell suspensions. Cells were stained with antibodies against CD4, CD8, MHC-II, CD44, and CD28 molecules for flow cytometry-assisted immunophenotyping analysis. The gating strategy included exclusion of doublet cells through forward and side scatters, height and width followed by gating on live cells (panel A). Live cell gating was furthermore used as a backbone population to obtain CD4+ and CD8+ cells (panel B), CD4+CD44+ cells (panel C), CD8+CD44+ cells (panel D), CD4+CD28+ cells (panel E), CD8+CD28+ cells (panel F), and MHC-II+ cells (panel G). Negative Ctrl-Control, unchallenged; Positive Ctrl-Control, unimmunized and challenged; D1-immunized with ntATX-D1 protein and challenged; D2—immunized with ntATX-D2 protein and challenged. Graphs show standard error of mean bars with the asterisks above the bars indicating statistical significance; *P<0.05, **P<0.01 or ***P<0.001.

FIGS. 9A-9B shows the serum antibody response in immunized turkeys. Turkeys were immunized with subunit recombinant proteins at 7, 8 and 9 weeks of age and the serum samples from birds (n=8) in the experimental groups were collected at two weeks post-last immunization prior to the C. septicum infection-challenge. The antigen-specific turkey IgY antibody levels were measured by ELISA using a two-fold serum dilution method. Negative Ctrl—Control; D1-immunized with ntATX-D1 protein; D2-immunized with ntATX-D2 protein.

FIGS. 10A-10F shows the histopathological changes in the skin of turkeys representing experimental groups. (FIG. 10A) Negative control. Occasional samples had focal, mild, often perivascular lymphocytic and heterophilic inflammation in the superficial dermis (arrowhead), affecting less than 5% of the tissue (lesion scores=2). Bar=150 μm. (FIG. 10B) Positive control. The dermis is markedly expanded by fibrin and edema (arrowhead), consistent with a lesion score of 4. Bar=325 μm. (FIG. 10C) ntATX-D1 vaccinated bird. Skin shows cell lysis of erythrocytes (arrowhead) multifocally affecting 30-75% of the tissue, consistent with a lesion score of 4. Bar=100 μm. (FIG. 10D) ntATX-D1 vaccinated bird. The deep dermis contains a discrete caseous granuloma characterized by an outer rim of macrophages and heterophils surrounding an inner, hypereosinophilic core of caseous necrosis. Bar=100 μm. (FIG. 10E) ntATX-D2 vaccinated bird. The deep dermal collagen is separated by abundant fibrin and edema, with small numbers of scattered heterophils. Bar=50 μm. (FIG. 10F) ntATX-D2 vaccinated bird. Severe cell lysis of erythrocytes within blood vessels, with marked expansion of the adjacent deep dermis by abundant fibrin and edema. Bar=50 μm.

FIGS. 11A-11F show the histopathological changes in the muscle of turkeys representing experimental groups. (FIG. 11A) Negative control. No lesions were present within any of the negative control birds, with scores equivalent to 1 for all categories. Bar=50 μm. (FIG. 11B) Positive control. Skeletal myofibers often exhibit monophasic sarcoplasmic fragmentation and hypereosinophilia with loss of cross-striations, affecting over 75% of the myofibers (lesion score=5). Myofibers are often separated by fibrin and edema (lesion score=4) admixed with scattered cellular lysis, affecting 30-75% of the tissue. An inflammatory infiltrate is absent. Bar=50 μm. (FIG. 11C) Positive control. Foci of cell lysis, fibrin, and edema frequently contain abundant large bacterial rods. Lesion score was 4 for bacteria, with bacteria scattered extensively throughout 30-75% of the section. Bar=10 μm. (FIG. 11D) ntATX-D1 vaccinated bird. Skeletal myofibers are replaced by a focally extensive caseous granuloma, characterized by an outer rim of abundant macrophages, multinucleated giant cells, and fewer heterophils and lymphocytes (granuloma lesion score=4). The rim surrounds an inner core of caseous necrosis of skeletal muscle, with abundant cellular debris and brightly eosinophilic, necrotic myofibers (caseous necrosis lesion score=4). Bar=50 μm. (FIG. 11E) ntATX-D1 vaccinated bird. Erythrocytes within and surrounding vessels exhibit cell lysis multifocally in up to 25% of the section, consistent with a cell lysis lesion score of 3 (arrowhead). Myofibers exhibit similar degeneration and necrosis as in the positive controls. Fibrin and edema multifocally separate myofibers. Bar=50 μm. (FIG. 11F) ntATX-D2 vaccinated bird. Similar cell lysis, fibrin, edema, and myopathy are visualized as described in the positive control and D1 vaccinated bird. Bar=50 μm.

FIGS. 12A-12D show the histopathological changes in the spleen of turkeys representing experimental groups. (FIG. 12A) Negative control. No lesions are identified with the negative control group. The arrowhead represents a bursa dependent lymphoid nodule. (FIG. 12B) Positive control. A subset of birds had unapparent bursal dependent nodules, depletion of lymphocytes around sheathed venules, and reticular hyperplasia. Scores in this animal for lymphoid depletion and reticular hyperplasia (arrow) were a 4 with 30-75% of the tissue involved by multifocal lesions. (FIG. 12C) ntATX-D1 vaccinated bird. Occasional birds had atrophy of bursal dependent lymphoid nodules (arrowhead) and of lymphocytes around sheathed venules (lymphoid depletion lesion score=3). Reticular hyperplasia was equivalent of a lesion score 4, affecting 30-75% of the tissue. (FIG. 12D) ntATX-D2 vaccinated bird. Occasional birds had lymphoid depletion (arrowhead) and reticular hyperplasia (arrow). Bars=50 μm.

FIG. 13 shows the cloning strategy. Two non-toxic regions of the alpha toxin (ATX) sequence, devoid of critical sites for toxicity, were identified and amplified from Clostridium septicum using specific primers. The resulting gene fragments ntATX-D1 and ntATX-D2, were digested with restriction enzymes and subsequently cloned into the NICE expression vector pNZ8124. The recombinant pNZ8124 plasmids were initially transformed into the recA+ E. coli strain MC106, and then introduced into Lactococcus lactis subsp. cremoris strain NZ9000 for Nisin-induced gene expression.

FIGS. 14A-14C show cloning, expression and identification of ntATX-D1 and ntATX-D2 in Lactococcus lactis. Panel A: Genes encoding the ntATX-D1 and ntATX-D2 domains were amplified from C. septicum and cloned into the pNZ8124 vector. The recombinant pNZ8124 constructs were then transformed into L. lactis NZ9000. To verify successful transformation, colony PCR was performed using the ntATX-D1/D2 domain-specific primers. L-1Kb+ DNA ladder, D1-Amplicon from ntATX-D1+pNZ8124+L.lactis (912 bp), D2-Amplicon from ntATX-D2+ pNZ8124+L.lactis (531 bp), NCx-Amplicon from empty pNZ8124 vector+L. lactis. Panel B: RNA extracted from the ntATX-D1/D2_pNZ8124 transformed L. lactis cells was reverse transcribed to cDNA, and PCR was performed with specific internal primers to verify the expression of the ntATX domains. L-100 bp DNA ladder, D1-t-Amplicon from ntATX-D1 cDNA, D1-p-Positive Control (Amplicon from rpNZ8124_ntATX-D1), D2-t-Amplicon from ntATX-D2 cDNA, D2-p-Positive Control (Amplicon from rpNZ8124_ntATX-D2), NCx-PCR negative control. Panel C: Western Blot analysis of concentrated supernatants after nisin induction, confirms the surface expression of ntATX-D1 (˜42 kDa) and ntATX-D2 (˜25 kDa) in L. lactis. L-Protein ladder, Lane 1—ntATX-D1+pNZ8124+L.lactis concentrated supernatant after nisin induction, Lane 2-ntATX D1 protein (PCx), Lane 3-empty pNZ8124 vector+L. lactis concentrated supernatant (NCx), Lane 4-ntATX-D2+pNZ8124+L.lactis concentrated supernatant after nisin induction, Lane 5-ntATX D2 protein (PCx).

FIGS. 15A-15B show mortality and gross pathology lesions in turkeys following C. septicum challenge. The birds were immunized with the oral probiotic vaccine at 7, 8, and 9 weeks of age. One week following the final immunization, the birds were experimentally infected with Clostridium septicum Strain B1. Clinical signs and mortality were monitored over a 72-hours post infection, and gross lesions were assessed and scored during necropsy. The study groups included: NCx-negative control, unchallenged (n=10); PCx-positive control, which received pNZ8124 (empty vector without insert)+Lactococcus lactis and challenged (n=11); D1-immunized with L. lactis+pNZ8124+ntATX-D1 and challenged (n=13); D2-immunized L. lactis+pNZ8124+ntATX-D2 and challenged (n=12). Bars represent the median score with range, and asterisks above the bars denote statistical significance compared to the PCx group: ***P<0.001.

FIGS. 16A-16C show histopathological changes observed in turkeys immunized and subsequently challenged with C. septicum. Panel A represents cutaneous lesions characterized by inflammation (including fibrin or edema, heterophils, and lymphocytic or histiocytic inflammation), gangrenous dermatitis (including fibrin or edema, cell lysis, and bacteria), and caseous necrosis/granuloma lesions. Panel B represents skeletal muscle lesions composed of inflammation, gangrenous dermatitis, caseous necrosis/granuloma lesions, or myopathy. Panel C represents splenic histopathologic lesions characterized by splenic necrosis and cell lysis or lymphoid depletion and reticular hyperplasia. NCx-negative control, unchallenged (n=10); PCx-positive control, which received pNZ8124 (empty vector without insert)+L. lactis and challenged (n=11); D1-immunized with L. lactis+pNZ8124+ntATX-D1 (n=13); D2-immunized with L. lactis+pNZ8124+ntATX-D2 (n=12). Groups are represented with bars indicating median scores with range. Asterisks denote statistical significance: *P<0.05, **P<0.01, or ***P<0.001.

FIG. 17 shows immune gene expression in skin tissue of immunized turkeys. Turkeys were immunized with food-grade oral probiotic vaccine at 7, 8, and 9 weeks of age, followed by a Clostridium septicum challenge one week after the final immunization. Tissue samples from birds in the experimental groups were collected at termination for RNA extraction and cDNA synthesis.

Real-time PCR was performed to quantify the expression of the genes indicated in the figure, with expression levels shown relative to the β-actin reference gene. The experimental groups were as follows: Negative Control-unchallenged; Positive Control-received pNZ8124 (empty vector without insert)+Lactococcus lactis and challenged; D1-immunized with pNZ8124+ntATX-D1+Lactococcus lactis and challenged; D2-immunized with pNZ8124+ntATX-D2+Lactococcus lactis and challenged. The graphs display standard error of the mean bars, with asterisks above the bars indicating statistical significance (*P<0.05 and ***P<0.001).

FIG. 18 shows immune gene expression in muscle tissue of immunized turkeys. Turkeys were immunized with food-grade oral probiotic vaccine at 7, 8 and 9 weeks of age followed by a C. septicum challenge one-week post-last immunization. Tissue samples from birds in the experimental groups were collected at termination for RNA extraction and cDNA synthesis. Real-time PCR to quantify the expression of genes indicated in the figure was performed and the expression levels are shown as relative to β-actin reference gene. Negative Control-unchallenged; Positive Control-received pNZ8124 (empty vector without insert)+Lactococcus lactis and challenged; D1-immunized with pNZ8124+ntATX-D1+Lactococcus lactis and challenged; D2-immunized with pNZ8124+ntATX-D2+Lactococcus lactis and challenged. Graphs show standard error of mean bars with the asterisks above the bars indicating statistical significance: *P<0.05, or **P<0.01.

FIG. 19 shows immune gene expression in spleen tissue of immunized turkeys. Turkeys were immunized with food-grade oral probiotic vaccine at 7, 8, and 9 weeks of age, followed by a Clostridium septicum challenge one week after the final immunization. Tissue samples from birds in the experimental groups were collected at termination for RNA extraction and cDNA synthesis. Real-time PCR was performed to quantify the expression of the genes indicated in the figure, with expression levels shown relative to the β-actin reference gene. The experimental groups were as follows: Negative Control-unchallenged; Positive Control-received pNZ8124 (empty vector without insert)+Lactococcus lactis and challenged; D1-immunized with pNZ8124+ntATX-D1+Lactococcus lactis and challenged; D2-immunized with pNZ8124+ntATX-D2+Lactococcus lactis and challenged. The graphs display standard error of the mean bars, with asterisks above the bars indicating statistical significance (*P<0.05).

FIG. 20 shows immune gene expression in Cecal Tonsil tissue of immunized turkeys. Turkeys were immunized with food-grade oral probiotic vaccine at 7, 8 and 9 weeks of age followed by a C. septicum challenge at one-week post-last immunization. Tissue samples from birds in the experimental groups were collected at termination for RNA extraction and cDNA synthesis. Real-time PCR to quantify the expression of genes indicated in the figure was performed and the expression levels are shown as relative to β-actin reference gene. Negative Ctrl-Control, unchallenged; Positive Ctrl-received pNZ8124 (empty vector without insert)+Lactococcus lactis and challenged; D1-immunized with pNZ8124+ntATX-D1+Lactococcus lactis and challenged; D2-immunized with pNZ8124+ntATX-D2+Lactococcus lactis and challenged. Graphs show standard error of mean bars with the asterisks above the bars indicating statistical significance: *P<0.05, **P<0.01 or ***P<0.001.

FIGS. 21A-21B show serum antibody responses in immunized turkeys. Turkeys were immunized with food-grade oral probiotic vaccine at 7, 8 and 9 weeks of age and the serum samples from birds in the experimental groups were collected at one-week post-last immunization prior to the C. septicum challenge. The antigen-specific turkey IgY antibody levels were measured by ELISA using a two-fold serum dilution method. Control-Negative control, unchallenged; D1-immunized with pNZ8124+ntATX-D1+Lactococcus lactis and challenged; D2-immunized with pNZ8124+ntATX-D2+Lactococcus lactis and challenged. Statistical significance is indicated as: *P<0.05 and **P<0.01.

FIGS. 22A-22C show the recovery of Lactococcus lactis from immunized turkeys; The ntATX-D1 (Panel A) and ntATX-D2 (Panel B) domains were successfully amplified from isolated Lactococcus lactis colonies obtained from the intestinal contents of vaccinated birds. To verify the strain of the isolated colonies cultured on selective agar, 16S rRNA PCR was performed. This yielded a 348 bp amplicon on the agarose gel, confirming the strain as L. lactis (Panel C). Panel A: L-100 bp DNA ladder, NCx—PCR Negative Ctrl, D1 Ctrl—PCR ntATX-D1 Ctrl, D1-ntATX-D1 (912 bp) amplified from isolated Lactococcus lactis colonies obtained from the intestinal contents of pNZ8124+ntATX-D1+Lactococcus lactis vaccinated birds. Panel B: L-100 bp DNA ladder, NCx-PCR Negative Ctrl, D2-ntATX-D2 (531 bp) amplified from isolated Lactococcus lactis colonies obtained from the intestinal contents of pNZ8124+ntATX-D2+Lactococcus lactis vaccinated birds, D2 Ctrl-PCR D2 Ctrl. Panel C: L-100 bp DNA ladder, Lane 1,2-Amplicon from colonies obtained from the intestinal contents of pNZ8124+ntATX-D1+Lactococcus lactis vaccinated birds, Lane 3,4-Amplicon from colonies obtained from the intestinal contents of pNZ8124+ntATX-D2+Lactococcus lactis vaccinated birds, Lane 5,6-Amplicon from colonies obtained from the intestinal contents of positive control birds which received empty pNZ8124+Lactococcus lactis, Lane 7: PCR negative control.

FIGS. 23A-23D show photomicrographs of skin tissues from turkeys representing different experimental groups. A) NCx bird with no histologic abnormalities. Bar=900 μm. B) D1-immunized bird with mild edema and perivascular aggregates of heterophils, lymphocytes, and histiocytes. C) D2-immunized bird with mild edema and rare perivascular lymphocytes and histocytes. Bar=900 μm. D) PCx bird with moderate edema and heterophils scattered throughout the subcutis with multifocal perivascular aggregates of lymphocytes and histiocytes. Bar=400 μm. NCx-negative control, unchallenged (n=10); PCx—positive control, which received pNZ8124 (empty vector without insert)+L. lactis and challenged (n=11); D1-immunized with L. lactis+pNZ8124+ntATX-D1 (n=13); D2-immunized with L. lactis+pNZ8124+ntATX-D2 (n=12).

FIGS. 24A-24D show photomicrographs of skeletal muscle tissues from turkeys representing different experimental groups. A) Negative control bird with normal skeletal myofibers and no lesions identified. Bar=100 μm. B) D1-immunized bird with focally extensive granuloma formation surrounded by a rim of heterophils, macrophages, and reactive fibroblasts and vessels. Bar=50 μm. C) D2-immunized bird with scattered perivascular aggregates of lymphocytes and macrophages. Bar=100 μm. D) Positive control bird with degenerate skeletal myofibers, edema, and cell lysis. Bar=50 μm. NCx-negative control, unchallenged (n=10); PCx-positive control, which received pNZ8124 (empty vector without insert)+L. lactis and challenged (n=11); D1-immunized with L. lactis+pNZ8124+ntATX-D1 (n=13); D2-immunized with L. lactis+pNZ8124+ntATX-D2 (n=12).

FIGS. 25A-25D show photomicrographs of spleen tissues from turkeys representing different experimental groups. Bars=50 μm. A) Negative control, B) D1-immunized, and C) D2-immunized birds had no lesions identified within their groups (lesion scores=1). D) Positive control birds occasionally had atrophy of lymphocytes surrounding sheathed vessels and of bursal dependent lymphoid nodules as pictured here. Scores in this spleen were 4 for lymphoid depletion and reticular hyperplasia with 30-75% of the tissue involved. NCx-negative control, unchallenged (n=10); PCx-positive control, which received pNZ8124 (empty vector without insert)+L. lactis and challenged (n=11); D1-immunized with L. lactis+pNZ8124+ntATX-D1 (n=13); D2-immunized with L. lactis+pNZ8124+ntATX-D2 (n=12).

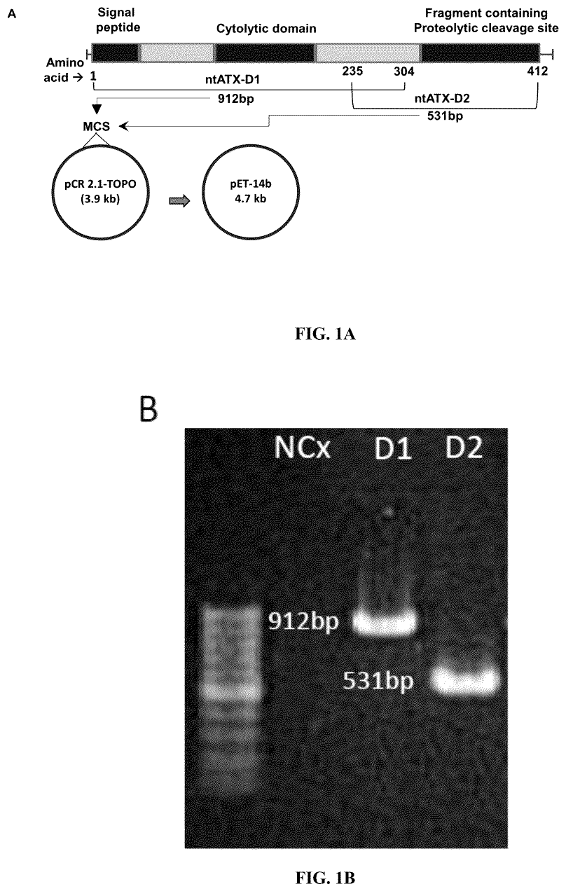

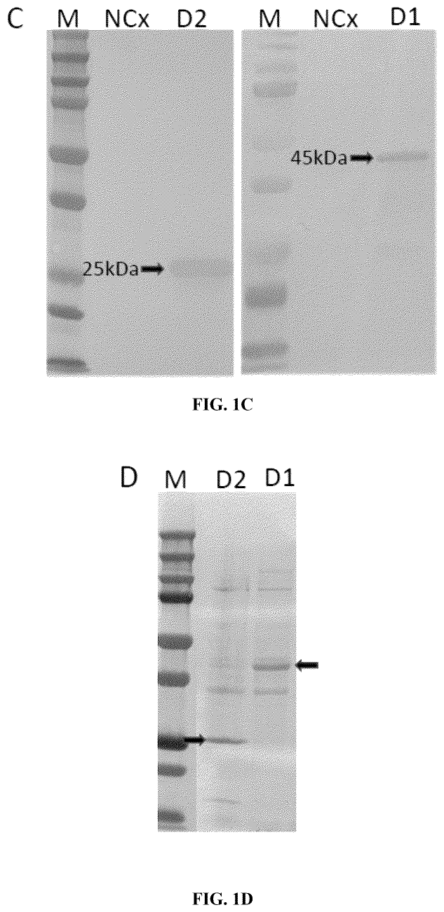

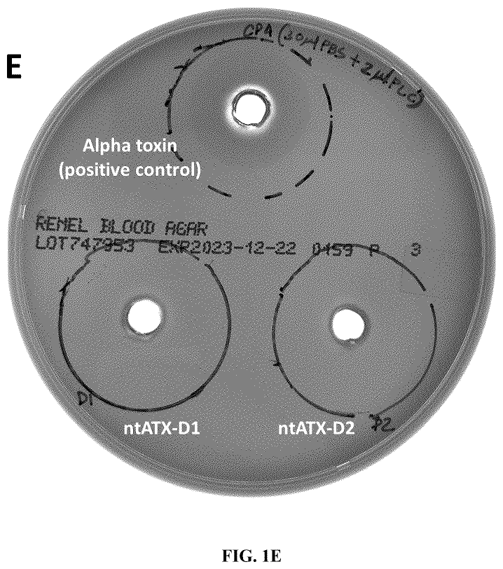

FIG. 26 shows the study design. Three experimental groups received different treatments as shown in the table in FIG. 26. Broiler chickens were immunized at days 7 (prime) and 14 (boost) of age and infected/challenged with virulent C. septicum Str. B1 at one week following the booster immunization. Birds were monitored for 72 hours for mortality and clinical signs, followed by necropsy. Protection assessment included evaluating changes in body weights during the challenge period and gross pathology lesions followed by immune gene expression in tissues, cellular responses and serology, as indicated in FIG. 1.

FIGS. 27A-27B shows body weight changes and gross pathology lesions in chickens following C. septicum challenge. Broiler chickens were immunized with subunit recombinant ntATX-D2 protein at 7 and 14 days of age followed by a C. septicum challenge at one-week post-last immunization. Changes in the body weight gain during the 72 hours challenge period and the gross lesions were scored at necropsy. Neg Ctrl-negative control, unvaccinated and unchallenged (n=10); Pos Ctrl-positive control, unimmunized and challenged (n=15); D2-immunized with ntATX-D2 protein and challenged (n=15). Bars represent median score with range and the asterisks above the bars indicate statistical significance, when compared to PCx group: *P<0.05, P<0.01 or ***P<0.001.

FIGS. 28A-28B shows gross pathology lesions in unvaccinated versus vaccinated chickens.

FIG. 29 shows immune gene expression in skin tissue of immunized chickens following C. septicum challenge. Broiler chickens were immunized with subunit recombinant ntATX-D2 protein at 7 and 14 days of age followed by a C. septicum challenge at one-week post-last immunization. Tissue samples from birds in the experimental groups were collected at termination (day 24 of age) for RNA extraction and cDNA synthesis. Real-time PCR to quantify the expression of genes indicated in the figure was performed and the expression levels are shown as fold changes relative to β-actin reference gene. Neg Ctrl-negative control, unvaccinated and unchallenged (n=8); Pos Ctrl-positive control, unimmunized and challenged (n=10); D2-immunized with ntATX-D2 protein and challenged (n=10). Graphs show standard error of mean bars with the asterisks above the bars indicating statistical significance. *P<0.05 or **P<0.01.

FIG. 30 shows immune gene expression in muscle tissue of immunized in chickens following C. septicum challenge. Broiler chickens were immunized with subunit recombinant ntATX-D2 protein at 7 and 14 days of age followed by a C. septicum challenge at one-week post-last immunization. Skeletal muscle tissue samples from birds in the experimental groups were collected at termination (day 24 of age) for RNA extraction and cDNA synthesis. Real-time PCR to quantify the expression of genes indicated in the figure was performed and the expression levels are shown as fold changes relative to β-actin reference gene. Neg Ctrl-negative control, unvaccinated and unchallenged (n=8); Pos Ctrl-positive control, unimmunized and challenged (n=10); D2-immunized with ntATX-D2 protein and challenged (n=10). Graphs show standard error of mean bars with the asterisks above the bars indicating statistical significance. *P<0.05 or **P<0.01.

FIG. 31 shows immune gene expression in spleen tissue of immunized in chickens following C. septicum challenge. Broiler chickens were immunized with subunit recombinant ntATX-D2 protein at 7 and 14 days of age followed by a C. septicum challenge at one-week post-last immunization. Tissue samples from birds in the experimental groups were collected at termination (day 24 of age) for RNA extraction and cDNA synthesis. Real-time PCR to quantify the expression of genes indicated in the figure was performed and the expression levels are shown as fold changes relative to β-actin reference gene. Neg Ctrl-negative control, unvaccinated and unchallenged (n=8); Pos Ctrl-positive control, unimmunized and challenged (n=10); D2-immunized with ntATX-D2 protein and challenged (n=10). Graphs show standard error of mean bars with the asterisks above the bars indicating statistical significance. *P<0.05.

FIGS. 32A-32B shows the serum antibody response in immunized chickens. Broiler chickens were immunized with subunit recombinant ntATX-D2 protein at 7 and 14 days of age followed by a C. septicum challenge at one-week post-last immunization and serum samples from birds in the experimental groups were collected. The antigen-specific turkey IgY antibody levels were measured by ELISA using a two-fold serum dilution method. Neg Ctrl-negative control, unvaccinated and unchallenged (n=8); Pos Ctrl-positive control, unimmunized and challenged (n=10); D2—immunized with ntATX-D2 protein and challenged (n=10). Bars represent median score with range and the asterisks above the bars indicate statistical significance, when compared to PCx group: *P<0.05.

FIG. 33 shows gating strategy used for cellular immunophenotyping. The gating strategy included exclusion of doublet cells through forward and side scatters, height and width followed by gating on live cells. Live cell gating was furthermore used as a backbone population to obtain CD3+TCRγδ cells, IgM+ B cells and KUL-01+MHC-II+ macrophages. The backbone gate for T cells was derived from the live CD3+ population, which was used to obtain CD4+ and CD8+ T cells, as well as CD4+CD28+ T and CD8+CD28+ T cells.

FIG. 34 shows peripheral blood and splenic cellular responses in immunized chickens following C. septicum challenge. Broiler chickens were immunized with subunit recombinant ntATX-D2 protein at 7 and 14 days of age followed by a C. septicum challenge at one-week post-last immunization. Blood and spleen samples from birds in the experimental groups were collected at termination (day 24 of age) for the preparation of single cell suspensions. Cells were stained with antibodies against chicken CD3, CD4, CD8, KUL-01, MHC-II, TCRγδ, IgM and CD28 molecules for flow cytometry-assisted immunophenotyping analysis. Neg Ctrl-negative control, unvaccinated and unchallenged (n=8); Pos Ctrl-positive control, unimmunized and challenged (n=10); D2—immunized with ntATX-D2 protein and challenged (n=10). Graphs show standard error of mean bars with the asterisks above the bars indicating statistical significance. *P<0.05, **P<0.01 or ****P<0.0001.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

The term “variant” means a polypeptide derived from a parent albumin by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X−1’ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit or more biological activities associated with binding a ligand and/or binding DNA at a specific binding site.

Variants comprising a fragment of a reference amino acid sequence are contemplated herein. A “fragment” is a portion of an amino acid sequence which is identical in sequence to but shorter in length than the reference sequence. A fragment may comprise up to the entire length of the reference sequence, minus at least amino acid residue. For example, a fragment may comprise from 5 to 1000 contiguous amino acid residues of a reference polypeptide. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous amino acid residues of a reference polypeptide. Fragments may be preferentially selected from certain regions of a molecule, for example the N-terminal region and/or the C-terminal region of a polypeptide. The term “at least a fragment” encompasses the full-length polypeptide.

The word “vector” refers to any vehicle that carries a polynucleotide into a cell for the expression of the polynucleotide in the cell. The vector may be, for example, a plasmid, a virus, a phage particle, or a nanoparticle. Once transformed into a suitable host, the vector may replicate and function independently of the host genome, or may in some instances, integrate into the genome itself. In some embodiments, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation. In other embodiments, the vector is a lipid nanoparticle. Lipid nanoparticles can be used to deliver mRNA to a host cell for expression of the mRNA in the host cell. In some embodiments, the expression vector comprises a plasmid or a virus or viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding a chimeric polypeptide is a naked DNA or is comprised in a nanoparticle (e.g., liposomal vesicle, porous silicon nanoparticle, gold-DNA conjugate particle, polyethyleneimine polymer particle, cationic peptides, etc.).

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

A “vaccine” refers to a biological preparation that provides active acquired immunity to a particular infectious diseases caused by a virus, bacteria, parasite, or any other microorganisms. Vaccines typically comprise an agent or several agents, also referred to as antigens, resembling the disease-causing microorganism and is often made from weakened or killed forms of the microbe, its toxins, or its surface proteins/peptides. Vaccines are also made to comprise additional components, such as adjuvants, preservatives, and/or stabilizers to boost the immune response, improve safety, and improve vaccine storage.

The term “kit” describes a wide variety of bags, containers, carrying cases, and other portable enclosures which may be used to carry and store solid substances, liquid substances, and other accessories necessary to administer the vaccine composition to a subject or express the nucleic acid sequence encoding the non-toxic domains disclosed herein.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

An “adjuvant” refers to a drug, molecule, substance, or a combination thereof that is used to increase the efficacy or potency of certain therapeutic agents, such as for example vaccines and/or antibodies. “Adjuvant(s)” are often at least one ingredient used in some vaccines that help create a stronger immune response in the host receiving said vaccine.

### Vaccine Compositions, Kits, and Vectors

The present invention provides vaccine compositions and kits comprising a non-toxic domain of a toxin. The present disclosure also provides vectors comprising nucleic acid sequences encoding the non-toxic domain. Also disclosed herein are methods of using these vaccines and vectors, which are described in more detail below.

Clostridial/gangrenous dermatitis, or cellulitis. is a disease of poultry animals that causes severe economic losses in the poultry industry worldwide. This disease, also called blue wing disease in chickens and cellulitis in turkeys, is primarily caused by Clostridium septicum or Clostridium perfringens type A. These bacterial pathogens can act individually to cause disease or in combination with other bacterial pathogens including, but not limited to Clostridium sordellii, Clostridium novyi, Staphylococcus aureus, Staphylococcus xylosus, Staphylococcus epidermidis, Escherichia coli, Pasteurella multocida, Pseudomonas aeruginosa, Enterococcus faecalis, Proteus spp., Bacillus spp., Erysipelothrix rhusiopathiae, and Gallibacterium anatis var. haemolytica.

In poultry animals, clostridial/gangrenous dermatitis is characterized by congestion, hemorrhage, necrosis of the skin and subcutaneous tissue, edema and/or emphysema. In poultry animals, Clostridial/Gangrenous dermatitis primarily affects the breast, back, abdomen, thighs, tails, and wings areas. Thus, there remains a need to develop a vaccine composition, kit, and/or vector for preventing, decreasing, reducing, and/or ameliorating the spread of bacterial pathogens causing Clostridial/Gangrenous dermatitis and other related diseases in poultry animals.

In one aspect, disclosed herein is a vaccine composition comprising a non-toxic domain of an alpha toxin (ATX) protein and a pharmaceutically acceptable carrier, wherein the non-toxic domain comprises ntATX-D1, ntATX-D2, or a combination thereof. Also described herein is the use of immunogenic fragments of these domains. By “immunogenic fragment” is meant a fragment which is of sufficient length and composition to cause an immune response in an individual to which it is administered.

Clostridium septicum is a Gram-positive, anaerobic, spore forming and toxin-producing bacterium that expresses an alpha-toxin (ATX) protein that has been implicated as the key virulence factor in gangrenous dermatitis pathogenesis. Alpha-toxin (ATX) is a protein toxin that can be produced by bacteria (such as, for example, Clostridium septicum). ATX is a pore-forming protein that forms hexamers and inserts into cell membranes, thereby disrupting the cell membrane of cells and damaging tissue in infected subjects which leads to diseases (such as, for example, Clostridial dermatitis).

Two regions of ATX have been identified which were predicted to lack cellular toxicity. The first region, referred to as non-toxic ATX Domain 1 (ntATX-D1), a 912 bp segment (SEQ ID NO: 2), was devoid of the proteolytic cleavage site (Arg-367-Ser-368), which is critical for toxin's activity (Example 1). The second region, referred to as non-toxic ATX Domain 2 (ntATX-D2), a 531 bp segment (SEQ ID NO: 4), was devoid of a signal peptide sequence (residue 1-31) and the region corresponding to cytolytic, pore-forming region of ATX (residue 203-232) (Example 1).

In some embodiments, the non-toxic domain ntATX-DI comprises at least 50% sequence identity to SEQ ID NO: 2. In some embodiments, the non-toxic domain ntATX-D1 comprises at least 60% sequence identity to SEQ ID NO: 2. In some embodiments, the non-toxic domain ntATX-D1 comprises at least 70% sequence identity to SEQ ID NO: 2. In some embodiments, the non-toxic domain ntATX-D1 comprises at least 75% sequence identity to SEQ ID NO: 2. In some embodiments, the non-toxic domain ntATX-DI comprises at least 80% sequence identity to SEQ ID NO: 2. In some embodiments, the non-toxic domain ntATX-DI comprises at least 90% sequence identity to SEQ ID NO: 2. In some embodiments, the non-toxic domain ntATX-D1 comprises at least 95% sequence identity to SEQ ID NO: 2. In some embodiments, the non-toxic domain ntATX-DI comprises at least 99% sequence identity to SEQ ID NO: 2. In some embodiments, the non-toxic domain ntATX-D1 comprises SEQ ID NO: 2. Specifically, ntATX-D1 domain can be 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2, or any amount in-between, below, or above these ranges.

In some embodiments, the non-toxic domain ntATX-D2 comprises at least 50% sequence identity to SEQ ID NO: 4. In some embodiments, the non-toxic domain ntATX-D2 comprises at least 60% sequence identity to SEQ ID NO: 4. In some embodiments, the non-toxic domain ntATX-D2 comprises at least 70% sequence identity to SEQ ID NO: 4. In some embodiments, the non-toxic domain ntATX-D2 comprises at least 75% sequence identity to SEQ ID NO: 4. In some embodiments, the non-toxic domain ntATX-D2 comprises at least 80% sequence identity to SEQ ID NO: 4. In some embodiments, the non-toxic domain ntATX-D2 comprises at least 90% sequence identity to SEQ ID NO: 4. In some embodiments, the non-toxic domain ntATX-D2 comprises at least 95% sequence identity to SEQ ID NO: 4. In some embodiments, the non-toxic domain ntATX-D2 comprises at least 99% sequence identity to SEQ ID NO: 4. In some embodiments, the non-toxic domain ntATX-D2 comprises SEQ ID NO: 4. Specifically, ntATX-D2 domain can be 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4, or any amount in-between, below, or above these ranges.

In some embodiments, the vaccine composition comprises SEQ ID NO: 2 and SEQ ID NO: 4, or fragments thereof. In some embodiments, the vaccine composition comprises ntATX-D1 or ntATX-D2, or fragments thereof. In some embodiments, the vaccine composition comprises ntATX-D1 and ntATX-D2, or fragments thereof.

Herein, vaccine compositions can comprise at least one or more non-toxic domains of ATX, or immunogenic fragments thereof. The vaccine compositions disclosed herein can also be combined with additional live attenuated, fragmented, or inactivated pathogenic components to prevent, decrease, reduce, and/or ameliorate the spread of bacterial pathogens causing Clostridial/Gangrenous dermatitis and other related diseases in subjects, wherein the subject can be a poultry animal, such as, for example, chicken or turkey.

In some embodiments, the vaccine composition further comprises an additional toxin, or fragments thereof. In some embodiments, the vaccine composition further comprises an adjuvant. In some embodiments, the adjuvants include, but are not limited to mineral oil, vegetable oil, aluminum hydroxide, calcium phosphate, alginate, chitosan, or combinations thereof.

In some embodiments, the pharmaceutically acceptable carrier comprises an oil-in-water emulsion, a nano-emulsion, a nanoparticle, an excipient, a diluent, a salt, a buffer, a stabilizer, a lipid, or combinations thereof.

In one aspect, disclosed herein is a vector comprising a nucleic acid sequence encoding the non-toxic domain of any preceding aspect.

In some embodiments, the nucleic acid sequence encodes an alpha-toxin (ATX) protein. In some embodiments, the nucleic acid sequence encodes ntATX-D1, ntATX-D2, or a combination thereof. In some embodiments, the nucleic acid sequence encodes at least 50% sequence identity to SEQ ID NO: 1. In some embodiments, the nucleic acid sequence encodes at least 60% sequence identity to SEQ ID NO: 1. In some embodiments, the nucleic acid sequence encodes at least 70% sequence identity to SEQ ID NO: 1. In some embodiments, the nucleic acid sequence encodes at least 75% sequence identity to SEQ ID NO: 1. In some embodiments, the nucleic acid sequence encodes at least 80% sequence identity to SEQ ID NO: 1. In some embodiments, the nucleic acid sequence encodes at least 90% sequence identity to SEQ ID NO: 1. In some embodiments, the nucleic acid sequence encodes at least 95% sequence identity to SEQ ID NO: 1. In some embodiments, the nucleic acid sequence encodes at least 99% sequence identity to SEQ ID NO: 1. In some embodiments, the nucleic acid sequence encodes SEQ ID NO: 1. Specifically, the nucleic acid sequence can encode 80%, 81%, 82%, 83%, 84%, 85%, 86%, 87%, 88%, 89%, 90%, 91%, 92%, 93%, 94%, 95%, 96%, 97%, 98%, or 99% of SEQ ID NO: 1, or any amount in-between, below, or above these ranges.

In some embodiments, the nucleic acid sequence encodes at least 50% sequence identity to SEQ ID NO: 3. In some embodiments, the nucleic acid sequence encodes at least 60% sequence identity to SEQ ID NO: 3. In some embodiments, the nucleic acid sequence encodes at least 70% sequence identity to SEQ ID NO: 3. In some embodiments, the nucleic acid sequence encodes at least 75% sequence identity to SEQ ID NO: 3. In some embodiments, the nucleic acid sequence encodes at least 80% sequence identity to SEQ ID NO: 3. In some embodiments, the nucleic acid sequence encodes at least 90% sequence identity to SEQ ID NO: 3. In some embodiments, the nucleic acid sequence encodes at least 95% sequence identity to SEQ ID NO: 3. In some embodiments, the nucleic acid sequence encodes at least 99% sequence identity to SEQ ID NO: 3. In some embodiments, the nucleic acid sequence encodes SEQ ID NO: 3. Specifically, the nucleic acid sequence can encode 80%, 81%, 82%, 83%, 84%, 85%, 86%, 87%, 88%, 89%, 90%, 91%, 92%, 93%, 94%, 95%, 96%, 97%, 98%, or 99% of SEQ ID NO: 3, or any amount in-between, below, or above these ranges.

In some embodiments, the vector comprises a Nisin-Controlled gene Expression system (the NICE system). The NICE system is an efficient and promising gene expression system. It is based on the autoregulation mechanism of nisin biosynthesis in Lactococcus lactis. In this system, a membrane-located histidine kinase, NisK, senses the inducing signal by nisin and autophosphorylates, then transfers phosphorous group to an intracellular response regulator protein, NisR, which in turn activates the nisA promoter to express the downstream gene(s). The NICE system allows regulated overproduction of a variety of interest proteins by several Gram-positive bacteria, especially L. lactis. In some embodiments, disclosed herein the NICE system comprises pNZ8124. In some embodiments, the NICE system is food-grade. In some embodiments, the L. lactis expression strain, as disclosed herein, is L. lactis-NZ9000.

In some embodiments, the vector comprises a plasmid, a virus, a viral vector, or a nanoparticle. In some embodiments, the vector is naked DNA. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding a chimeric polypeptide is a naked DNA or is comprised in a nanoparticle (e.g., liposomal vesicle, porous silicon nanoparticle, gold-DNA conjugate particle, polyethyleneimine polymer particle, cationic peptides, etc.).

In one aspect, disclosed herein is a kit comprising the vaccine composition or vector of any preceding aspect.

### Methods of Preventing Clostridial/Gangrenous Dermatitis

The present invention provides methods of preventing Clostridial/Gangrenous dermatitis in a subject, including, but not limited to, poultry animals.

In one aspect, disclosed herein is a method of preventing Clostridial/Gangrenous dermatitis in a subject, the method comprises administering a pharmaceutically effective amount of the vaccine composition of any preceding aspect, wherein the vaccine composition comprises a non-toxic domain of an alpha toxin (ATX) protein and a pharmaceutically acceptable carrier, wherein the non-toxic domain comprises ntATX-D1, ntATX-D2, or a combination thereof.

In one aspect, disclosed herein is a method of preventing Clostridial/Gangrenous dermatitis in a subject, the method comprises administering a pharmaceutically effective amount of the vaccine composition of any preceding aspect, wherein the vaccine composition comprises a non-toxic domain of an alpha toxin (ATX) protein and a pharmaceutically acceptable carrier, wherein the non-toxic domain comprises ntATX-D1, ntATX-D2, or a combination thereof.

In some embodiments, as in any of the preceding aspects the vaccine composition comprises a vector, wherein the vector comprises a nucleic acid sequence encoding a non-toxic domain of an alpha toxin (ATX) protein, wherein the non-toxic domain comprises ntATX-D1, ntATX-D2, or a combination thereof.

In some embodiments, Clostridial/Gangrenous dermatitis is caused by a Clostridium bacterium including, but not limited to Clostridium septicum, Clostridium perfringens type A, Clostridium sordellii, or Clostridium novyi. In some embodiments, Clostridial/Gangrenous dermatitis is caused by a bacterium including, but not limited to Staphylococcus aureus, Staphylococcus xylosus, Staphylococcus epidermidis, Escherichia coli, Pasteurella multocida, Pseudomonas aeruginosa, Enterococcus faecalis, Proteus spp., Bacillus spp., Erysipelothrix rhusiopathiae, and Gallibacterium anatis var. haemolytica.

In some embodiments, the vaccine composition is administered subcutaneously. In some embodiments, the vaccine composition is administered orally. As disclosed herein, the vector comprises a Nisin-Controlled gene Expression system (the NICE system), wherein the NICE system comprises pNZ8124. In some embodiments, the NICE system is food-grade. In some embodiments, the L. lactis expression strain, as disclosed herein, is L. lactis-NZ9000. As used herein, L. lactis-NZ9000 is at a concentration of about 1×10⁹ CFU/mL.

In some embodiments, when administered orally, the vaccine composition comprises two doses. In some embodiments, when administered orally, the vaccine composition comprises three doses (first dose, second dose and a third dose). In some embodiments, the first dose comprises 3 ml of the vaccine composition. In some embodiments, the second dose comprises 4 ml of the vaccine composition. In some embodiments, the third dose comprises 5 ml of the vaccine composition. In some embodiments, the vaccine composition comprises the G+M17 media.

Further disclosed herein, administration of the vaccine composition produces at least 50% decrease in gene expression level of TLR21, IL-1β, IL-6, and IFNγ genes in a tissue sample from an immunized subject compared to an unimmunized control. In some embodiments, the vaccine composition produces at least 60% decrease in gene expression level of TLR21, IL-1β, IL-6, and IFNγ genes in a tissue sample from an immunized subject compared to an unimmunized control. In some embodiments, the vaccine composition produces at least 70% decrease in gene expression level of TLR21, IL-1β, IL-6, and IFNγ genes in a tissue sample from an immunized subject compared to an unimmunized control. In some embodiments, the vaccine composition produces at least 80% decrease in gene expression level of TLR21, IL-1β, IL-6, and IFNγ genes in a tissue sample from an immunized subject compared to an unimmunized control. In some embodiments, the vaccine composition produces at least 90% decrease in gene expression level of TLR21, IL-1β, IL-6, and IFNγ genes in a tissue sample from an immunized subject compared to an unimmunized control. In some embodiments, the vaccine composition produces at least 100% decrease in gene expression level of TLR21, IL-1β, IL-6, and IFNγ genes in a tissue sample from an immunized subject compared to an unimmunized control. Specifically, the vaccine composition produces at least 50%, 60%, 70%, 80%, 90%, or 100% decrease in gene expression level of TLR21, IL-1β, IL-6, and IFNγ genes in a tissue sample from an immunized subject compared to an unimmunized control, or any amount in-between, below, or above these ranges. In some embodiments, the tissue sample comprises skin, muscle, spleen or cecal tonsil (CT).

In some embodiments, the vaccine composition produces at least 20% increase in frequency of splenic CD4+ T cells, circulatory γδ T cells, B cells or a combination thereof in whole sera of an immunized subject compared to an unimmunized subject. In some embodiments, the vaccine composition produces at least 30% increase in frequency of splenic CD4+ T cells, circulatory γδ T cells, B cells or a combination thereof in whole sera of an immunized subject compared to an unimmunized subject. In some embodiments, the vaccine composition produces at least 40% increase in frequency of splenic CD4+ T cells, circulatory γδ T cells, B cells or a combination thereof in whole sera of an immunized subject compared to an unimmunized subject. In some embodiments, the vaccine composition produces at least 50% increase in frequency of splenic CD4+ T cells, circulatory γδ T cells, B cells or a combination thereof in whole sera of an immunized subject compared to an unimmunized subject. In some embodiments, the vaccine composition produces at least 60% increase in frequency of splenic CD4+ T cells, circulatory γδ T cells, B cells or a combination thereof in whole sera of an immunized subject compared to an unimmunized subject. In some embodiments, the vaccine composition produces at least 70% increase in frequency of splenic CD4+ T cells, circulatory γδ T cells, B cells or a combination thereof in whole sera of an immunized subject compared to an unimmunized subject. In some embodiments, the vaccine composition produces at least 80% increase in frequency of splenic CD4+ T cells, circulatory γδ T cells, B cells or a combination thereof in whole sera of an immunized subject compared to an unimmunized subject. In some embodiments, the vaccine composition produces at least 90% increase in frequency of splenic CD4+ T cells, circulatory γδ T cells, B cells or a combination thereof in whole sera of an immunized subject compared to an unimmunized subject. In some embodiments, the vaccine composition produces at least 100% increase in frequency of splenic CD4+ T cells, circulatory γδ T cells, B cells or a combination thereof in whole sera of an immunized subject compared to an unimmunized subject. Specifically, the vaccine composition produces at least 20%, 30%, 40%, 50%, 60%, 70%, 80%, 90% or 100% increase in frequency of splenic CD4+ T cells, circulatory γδ T cells, B cells or a combination thereof in whole sera of an immunized subject compared to an unimmunized subject, or any amount in-between, below, or above these ranges.

In some embodiments, the method produces at least 10% increase in body weight of an immunized subject compared to an unimmunized subject. In some embodiments, the method produces a 20% increase in body weight of an immunized subject compared to an unimmunized subject. In some embodiments, the method produces a 30% increase in body weight of an immunized subject compared to an unimmunized subject. Specifically, the method produces at least 10% 20%, or 30% increase in body weight of an immunized subject compared to an unimmunized subject, or any amount in-between, below, or above these ranges.

In some embodiments, the method produces at least 50% decrease in disease severity of an immunized subject compared to an unimmunized subject. In some embodiments, the method produces at least 60% decrease in disease severity of an immunized subject compared to an unimmunized subject. In some embodiments, the method produces at least 70% decrease in disease severity of an immunized subject compared to an unimmunized subject. In some embodiments, the method produces at least 80% decrease in disease severity of an immunized subject compared to an unimmunized subject. In some embodiments, the method produces at least 90% decrease in disease severity of an immunized subject compared to an unimmunized subject. In some embodiments, the method produces at least 100% decrease in disease severity of an immunized subject compared to an unimmunized subject. Specifically, the method produces at least 50%, 60%, 70%, 80%, 90% or 100% decrease in disease severity of an immunized subject compared to an unimmunized subject, or any amount in-between, below, or above these ranges.

In some embodiments, the vaccine composition produces at least 40% decrease in mortality and/or at least 20% decrease in gross pathology of an immunized subject compared to an unimmunized subject. In some embodiments, the vaccine composition produces at least 40% decrease in mortality of an immunized subject compared to an unimmunized subject. In some embodiments, the vaccine composition produces at least 50% decrease in mortality of an immunized subject compared to an unimmunized subject. In some embodiments, the vaccine composition produces at least 60% decrease in mortality of an immunized subject compared to an unimmunized subject. In some embodiments, the vaccine composition produces at least 70% decrease in mortality of an immunized subject compared to an unimmunized subject. In some embodiments, the vaccine composition produces at least 80% decrease in mortality of an immunized subject compared to an unimmunized subject. In some embodiments, the vaccine composition produces at least 90% decrease in mortality of an immunized subject compared to an unimmunized subject. In some embodiments, the vaccine composition produces at least 100% decrease in mortality of an immunized subject compared to an unimmunized subject. Specifically, the vaccine composition produces at least 40%, 50%, 60%, 70%, 80%, 90% or 100% decrease in mortality of an immunized subject compared to an unimmunized subject, or any amount in-between, below, or above these ranges.

In some embodiments, the vaccine composition produces at least 20% decrease in gross pathology of an immunized subject compared to an unimmunized subject. In some embodiments, the vaccine composition produces at least 30% decrease in gross pathology of an immunized subject compared to an unimmunized subject. In some embodiments, the vaccine composition produces at least 40% decrease in gross pathology of an immunized subject compared to an unimmunized subject. In some embodiments, the vaccine composition produces at least 50% decrease in gross pathology of an immunized subject compared to an unimmunized subject. In some embodiments, the vaccine composition produces at least 60% decrease in gross pathology of an immunized subject compared to an unimmunized subject. In some embodiments, the vaccine composition produces at least 70% decrease in gross pathology of an immunized subject compared to an unimmunized subject. In some embodiments, the vaccine composition produces at least 80% decrease in gross pathology of an immunized subject compared to an unimmunized subject. In some embodiments, the vaccine composition produces at least 90% decrease in gross pathology of an immunized subject compared to an unimmunized subject. In some embodiments, the vaccine composition produces at least 100% decrease in gross pathology of an immunized subject compared to an unimmunized subject. Specifically, the vaccine composition produces at least 20%, 30%, 40%, 50%, 60%, 70%, 80%, 90% or 100% decrease in gross pathology of an immunized subject compared to an unimmunized subject, or any amount in-between, below, or above these ranges. In some embodiments, the subject is but not limited to a poultry animal. In some embodiments, the poultry animal comprises turkeys, chickens, ducks, quail, or geese.

Also disclosed are methods of diagnosing the subject before treatment. A number of methods to diagnose CD are known to those of skill in the art.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Immunization of Turkeys with Clostridium septicum Non-Toxic Alpha Toxin Recombinant Subunit Protein can Confer Protection Against Experimental Clostridial Dermatitis

Clostridial dermatitis (CD), caused by Clostridium septicum, is an economically important emerging disease of turkeys. Currently, there are no effective vaccines for CD control. Here, two non-toxic domains of C. septicum alpha toxin, namely ntATX-D1 and ntATX-D2, were identified, cloned, and expressed in E. coli as recombinant proteins to investigate their use as potential vaccine candidates. Turkeys were divided into four groups: a negative control (NCx) that did not receive C. septicum challenge, while an adjuvant-only positive control (PCx) and two immunization groups, ntATX-D1 immunization (D1) and ntATX-D2 immunization (D2), received C. septicum challenge. Turkeys were immunized subcutaneously with 100 μg of protein at 7, 8 and 9 weeks of age along with an oil-in-water nano-emulsion adjuvant, followed by a challenge at 11 weeks of age. Protection assessment included monitoring mortality, examining gross and histopathological lesions in the skin, muscle and spleen tissues, and evaluating immune responses. Results showed that the PCx group had a 50% mortality rate, while only 13.3% of birds in the D1 and D2 groups died post-challenge. The severity of CD lesions was highest in the PCx group, while the D2-immunized birds had significantly lower lesions scores when compared to PCx. Gene expression analysis revealed that the birds in the PCx had significantly higher expression of pro-inflammatory cytokine genes in the skin, muscle and spleen than the NCx group, while the D2 group had significantly lower expression of these genes compared to PCx. Peripheral blood cellular analysis showed increased frequencies of activated CD4+ and CD8+ cells in the immunized groups, particularly in the D2 group. Additionally, D1 and D2-immunized turkeys developed antigen-specific serum IgY antibodies. These findings indicate that ntATX-D2 can be a promising vaccine candidate for protecting turkeys against CD, with immune protection involving activation of CD4+ and/or CD8+ cells and downregulation of CD-induced inflammation.

Introduction: Clostridial Dermatitis (CD), also referred to as Gangrenous dermatitis or Cellulitis, first reported in 1993, is a re-emerging economically devastating disease of poultry with its prevalence being on the rise in the past decade or so. In turkeys, CD usually peaks around 13-18 weeks of age and is characterized by subcutaneous edema and emphysema with skin lesions in the breast/inguinal area, sometime extending to underlying muscle tissue followed by necrotic dermatitis and sudden deaths. Although several etiological agents have been implicated, the two known Clostridial pathogens are C. perfringens and C. septicum; however, C. septicum has more often been isolated and identified as the primary etiological agent for CD in commercial turkeys. Several predisposing factors such as overcrowding, poor ventilation and immunosuppression are implicated in CD development. Despite good management practices coupled with antibiotic treatment, CD control has remained a challenge for the poultry industry. Unfortunately, there are no effective vaccines currently available; this is partly because of the complexity of CD pathogenesis, while largely due to a poor understanding of immunity to CD in poultry.

Clostridium septicum is a Gram-positive, anaerobic, spore forming and toxin-producing bacterium with alpha-toxin (ATX) being implicated as the key virulence factor in the disease pathogenesis. Two theories are currently available to explain CD pathogenesis; The ‘Inside out’ theory proposes that Clostridia, being ubiquitous and opportunistic in nature, can cause intestinal inflammation resulting in gut leakage, which in turn facilitate them to gain access, via the circulation, to the sites of skin abrasions or hypoxia, where they can multiply and produce toxins. The ‘outside in’ theory shows the entry of the agent into the subcutaneous tissues via skin wounds, where they multiply and produce CD. In support of the latter theory, it has been previously shown that turkeys inoculated with C. septicum subcutaneously can cause clinical CD, characterized by typical dermatitis lesions and related mortality.

Although several managemental practices such as good ventilation control, reducing overcrowding or timely vaccination of turkeys against immunosuppressive diseases have been tried with a variable degree of success, vaccination seems to offer a promising CD control strategy. Much of the vaccine work has focused on inducing antibodies against C. septicum ATX. Previous work showed that turkeys immunized subcutaneously with C. septicum bacterin-toxoid vaccine, under both laboratory and commercial setting, had significantly higher anti-C. septicum antibodies and reduced morbidity and mortality. In one of the two filed trials, C. septicum toxoid vaccine was shown to reduce CD-related mortality by about 50%. More recently, another study showed that adjuvantation of C. septicum bacterin-toxoid with a water-in-oil Montanide emulsion administered turkeys subcutaneously in commercial farm setting can reduce CD morbidity and mortality along with increased serum antibodies. Considering the critical role of alpha-toxin in CD pathogenesis and immunity, a previous study evaluated the efficacy of a noncytolytic C. septicum alpha-toxin (NCAT) peptide as a vaccine candidate in turkeys against CD when immunized subcutaneously. The immunization groups consisted of 1) purified NCAT, 2) crude NCAT, 3) Purified NCAT+C. septicum bacterin+alpha-toxoid, 4) Bacterin+alpha-toxoid, and 5) Unimmunized-challenged control. Results showed that while the group #4 showed highest efficacy, other treatment groups also showed a significant reduction in mortality to suggest that NCAT can be used as a vaccine candidate.

Herein, two non-toxic domains in ATX, namely ntATX-D1 and ntATX-D2, were cloned, expressed, and purified from E. coli as recombinant proteins. Turkeys were subcutaneously immunized with ntATX-D1 and ntATX-D2 proteins along with an oil-in-water nano emulsion to assess protection against CD using an experimental infection model. The protection assessment parameters included 1) Mortality and Gross pathology, 2) Histopathology of skin, muscle and spleen tissues, 3) Immune gene expression in skin, muscle, spleen and cecal tonsil (CT), 4) Peripheral blood cellular responses, and 5) Serum antibody evaluation.

### Materials and Methods

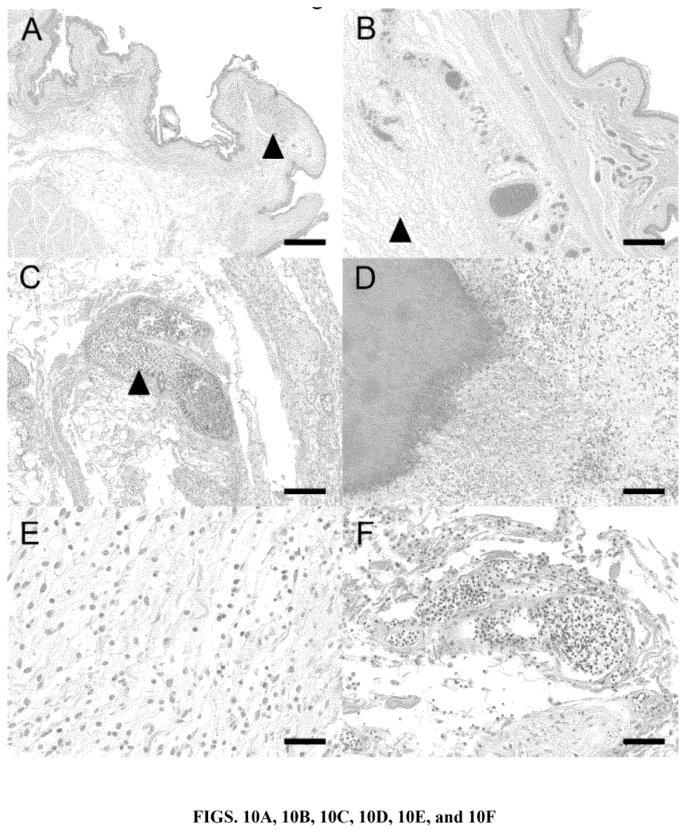

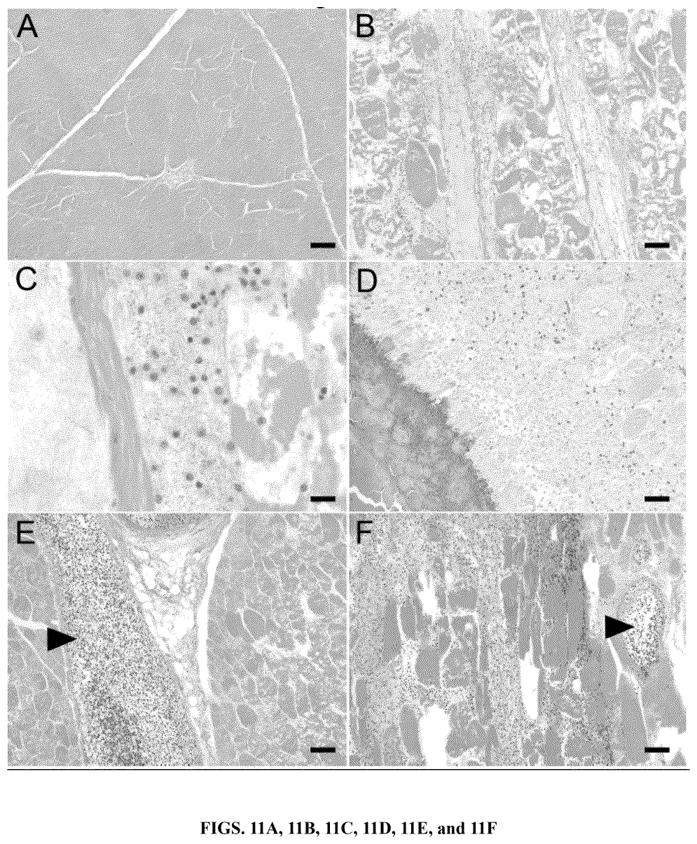

Cloning, expression and purification of recombinant proteins: Based on ATX sequence information, two regions were identified and predicted to lack cellular toxicity if cloned and expressed as recombinant proteins. The first region, referred to as non-toxic ATX Domain 1 (ntATX-D1), a 912 bp segment, was devoid of the proteolytic cleavage site (Arg-367-Ser-368), which is critical for toxin's activity, while the second region, referred to as non-toxic ATX Domain (ntATX-D2), as 531 bp segment, was devoid of a signal peptide sequence (residue 1-31) and the region corresponding to cytolytic, pore-forming domain of ATX (residue 203-232) (FIG. 1). The DNA sequence and the predicted amino acid sequence information related to ntATX-D1 and ntATX-D2 are given in FIG. 10. Genes encoding ntATX-D1 or ntATX-D2 were PCR amplified from the C. septicum str. B1 DNA using primers listed in Table 1. The PCR condition included 35 cycles of amplification with each cycle consisting of steps of denaturation (94° C. for 1 min), annealing (55° C. for 1 min), extension (72° C. for 1 min) and a final extension at 72° C. for 7 min. Amplified gene segments were then first cloned into the pCR 2.1 TA cloning vector (Invitrogen, Carlsbad, CA) followed by cloning into the pET14b expression vector (Novagen, Madison, WI) between the XhoI and BamHI restriction enzyme sites and transformed to E. coli DH5α host and then into E. coli BL21-DE3 expression host, as per the standard cloning protocol (TB055 pET System Manual). The N-terminal 6× histidine-tagged recombinant ntATX-D1 and ntATX-D2 proteins were purified using the ProBond Nickel-Chelating Resin columns (Invitrogen, Carlsbad, CA) and analyzed by SDS-PAGE. Immunoreactivity of these proteins to the anti-Histidine antibodies (Invitrogen, Carlsbad, CA) was confirmed by Western blot analysis. In instances when the protein purification yield was low due to the overexpression leading to the formation of inclusion bodies containing insoluble protein aggregates, solubilization and purification of ntATX-D1 and ntATX-D2 was carried out. The protein aggregates from E. coli expression host cell pellet were retrieved using inclusion body solubilization reagent (Pierce Protein Biology|Thermo Fisher Scientific, Rockford, IL) and the purity of the inclusion bodies was checked by SDS-PAGE before proteins were refolded via dialysis using Slide-A-Lyzer Cassette (ThermoFisherScientific). The dialyzed solution was concentrated using Amicon Ultra-15 Centrifugal filter units (Sigma-Aldrich) and the concentrated protein solution was run through the HisPur Ni-NTA columns (Thermo Scientific) to purify recombinant ntATX-D1 and ntATX-D2 proteins. The purification recovery of recombinant proteins from the columns was about 90%, which were then analyzed by gel electrophoresis, followed by confirming their immunoreactivity using Western blot analysis. In addition to immunoreactivity against anti-Histidine antibodies, purified ntATX-D1 and ntATX-D2 proteins were also reacted with serum collected CD-affected turkeys collected in the Western blot analysis. The primary serum antibody dilution of 1:100 followed by HRP-conjugated anti-turkey IgY antibody (Southern Biotech, Birmingham, AL) at a 1:500 dilution was used in immunoreactivity analysis.

To determine if the purified ntATX-D1 and ntATX-D2 were devoid of toxicity, the hemolytic activity of these proteins was evaluated using 5% defibrinated sheep blood agar plates. Briefly, about 5 mm diameter wells were punched into the agar followed by sealing the bottom with a thin layer of 1% agar. Wells were loaded with 30 μL of purified proteins (10 μg/well) and incubated at 37° C. overnight. The absence of zone of hemolysis was considered to determine the non-hemolytic activity of the proteins. As a positive control for hemolysis the purified C. perfringens hemolysin toxin, the alpha toxin also known as phospholipase C (Sigma-Aldrich, St. Louis, MO) was used.

Immunization, experimental challenge and sampling: Animal experimental protocols used in this research were approved by the North Carolina State University Institutional Animal Care and Use Committee (IACUC protocol 22-181-A). Six-week-old male turkeys were procured from Butterball Farms LLC. and were placed on fresh litter consisting of wood shavings in the animal rooms at the Laboratory Animal Research (Biosafety Level 2) facility of the College of Veterinary Medicine, North Carolina State University with unlimited access to water and non-medicated grower feed. The birds were individually identified with leg bands and were divided into four experimental groups with two control groups designated in relation to receiving or not receiving the C. septicum challenge. The first group termed as ‘negative control (NCx)’ had birds that did not receive the C. septicum challenge (n=10). However, this group included five birds that were immunized with ntATX-D1 protein and five birds immunized with ntATX-D2 protein. The second group termed as ‘positive control (PCx)’ had birds that received only the adjuvant but not the vaccine antigen but were challenged with C. septicum (n=13). The third group termed as ‘D1’ group had birds that were immunized with ntATX-D1 and challenged with C. septicum (n=15). The fourth group termed as ‘D2’ group had birds that were immunized with ntATX-D2 and challenged with C. septicum (n=15). Birds in the NCx, D1 and D2 groups received the 100 μg of ntATX-D1 or ntATX-D2 protein antigen via subcutaneous route in the inguinal skin fold on weeks 7, 8 and 9 of age along with the AddaVax (InvivoGen, San Diego, CA), an oil-in-water nano-emulsion vaccine adjuvant equivalent to MF59 at a ratio of 1:1 in a 0.2 mL volume. The NCx group birds were housed separately from the rest of the groups, while the PCx, D1 and D2 groups were housed in a room separated by an empty floor pen. The NCx group served as the control to evaluate the effect of C. septicum challenge in other groups as well as to determine immunization-related adverse effects, if any, while the PCx group provided a control to measure ntATX-D1 or ntATX-D2 vaccine-induced protective efficacy.

For experimental infection/challenge, C. septicum Str. B1 isolated from commercial turkeys affected with CD was used. C. septicum were grown in Reinforced Clostridial medium (Oxoid, Lenexa, KS) anaerobically at 37° C. for a period of 36-48 hours. Each bird in the infected group was given a dose of 2 mL (0.5×10⁸ CFU/mL) subcutaneously in the lower pectoral region with 1 mL on each side, while the unchallenged birds received growth medium only. Mortality was monitored for a period of 72 hours and the gross lesions at necropsy were scored as described below. Samples collected included skin, muscle and spleen for histology; skin, muscle, spleen and CT for immune gene expression; peripheral blood for immunophenotyping and serology.

Pathology: At necropsy, gross lesions characteristic of CD in the skin, muscle and spleen were examined and scored as 0=no lesions; 1=Dark red/purple-green discoloration, blisters on skin; 2=Cellulitis grade 1: moist/weepy skin with subcutaneous edema in the breast/thigh (lower abdomen) and lesions limited to smaller areas (1-3 cm) showing exudate with hemorrhagic foci; 3=Cellulitis grade 2: Larger areas (>3 cm) of subcutaneous lesions showing exudate accumulation accompanied by necrotic changes/emphysema/hemorrhage/gas-filled areas (crepitus); 4=Subcutaneous lesions extended to muscle with pale areas of discoloration and multifocal to coalescing hemorrhagic areas and necrotic changes; 5=Septicemia; Systemic organ involvement (spleen/liver/heart/others) showing marked discoloration, necrotic changes.

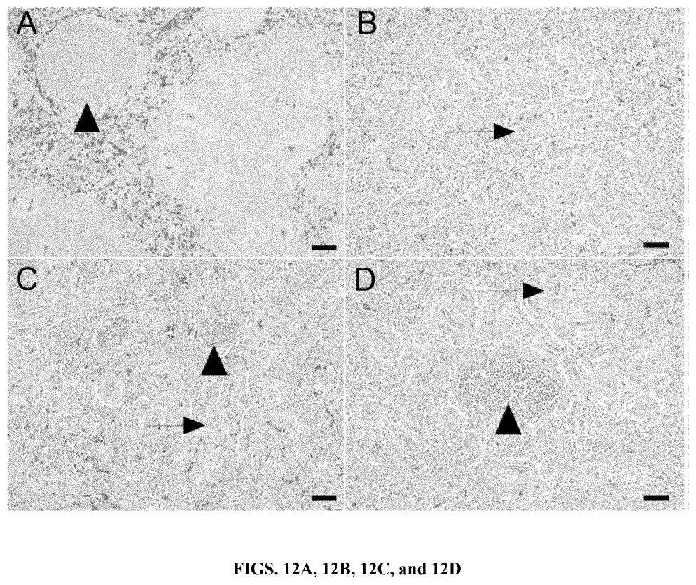

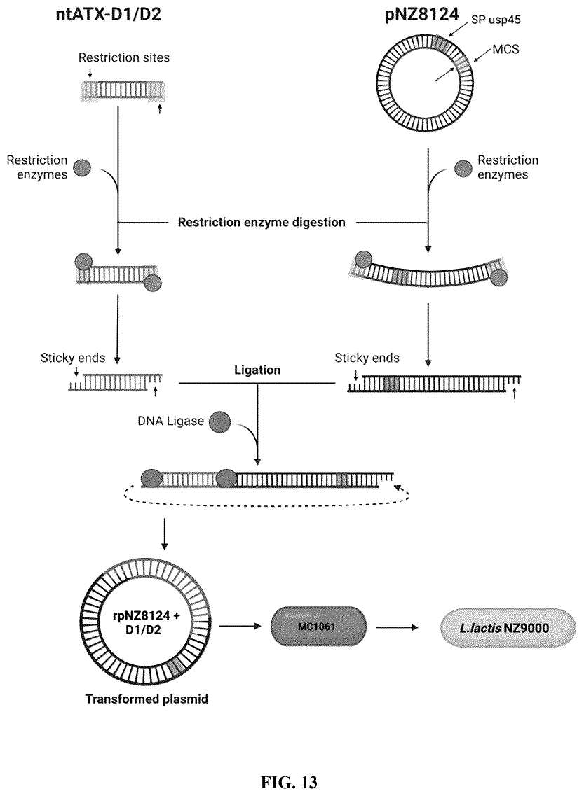

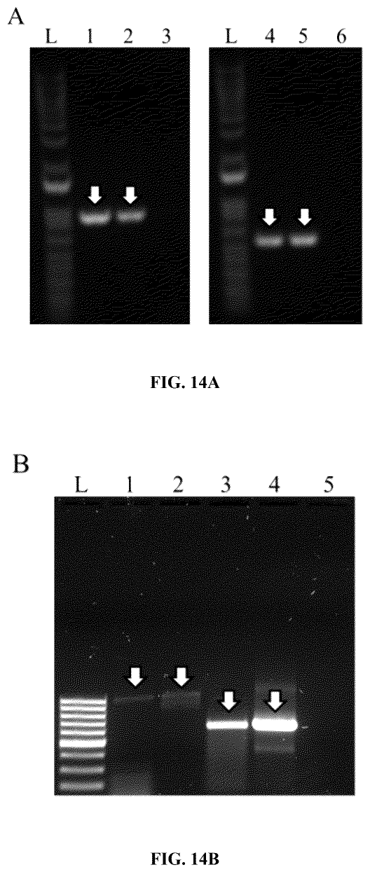

For histopathology, skin, skeletal muscle and spleen samples from grossly affected areas, if present, were examined from birds found moribund and euthanized during the clinical monitoring period of the study as well as those necropsied at termination. Tissues were fixed in 10% neutral buffered formalin for a minimum of 24 h, processed, sectioned at 5 μm, and stained with hematoxylin and eosin. Sections were examined and lesions were scored by the ACVP (American College of Veterinary Pathologists) boarded veterinary pathologists, and the select sections were additionally stained with Gram stain to identify Gram-positive Clostridium rods. Briefly, changes in the skin and muscle tissues were evaluated based on the criteria of inflammation (including fibrin/edema, heterophils, and lymphocytes/histocyte lesions), gangrenous dermatitis (including lesions of fibrin/edema, cell lysis, and bacteria) and caseous necrosis/granuloma lesions, while splenic changes were evaluated based on the necrosis and cell lysis, and splenic lymphoid depletion and hyperplasia. Each lesion was scored as 0=no lesion; 1=focal lesion and limited with <5% of tissue involved; 2=multifocal lesion scattered with 5-25% of tissue involved; 3=multifocal lesion extensive with 30-70% tissue involved; 5=diffuses lesions with >75% of tissue involved. Histology section images representing lesions scores in the skin, muscle and spleen are shown in FIGS. 11, 12, and 13, respectively.

Quantitative real-time PCR: Skin, muscle, cecal tonsils and spleen were collected in RNAlater solution (Invitrogen, Carlsbad, CA) from turkeys in all the groups (n=8) and stored at −80° C. until processing. Total RNA was extracted using a Bead Ruptor Elite Bead Mill Homogenizer (OMNI International, Kennesaw, GA) using 1.4 mm Ceramic Beads (OMNI International, Kennesaw, GA) suspended in TRIzol® reagent (Invitrogen, Carlsbad, CA) according to the manufacturer's protocol before being treated with a DNA-free Kit (Invitrogen, Carlsbad, CA). Subsequently, cDNA synthesis was performed with 500-1000 ng of purified RNA using a High-Capacity RNA-to-cDNA kit (Applied Biosystems, Waltham, MA) according to the manufacturer's recommended protocol. The resulting cDNA was subsequently diluted 1:10 in nuclease-free water for real-time PCR analysis.

Quantitative real-time PCR using SYBR Green was performed on diluted cDNA using a QuantStudio 6 Flex System and QuantStudio Real-Time PCR Software (Applied Biosystems, Waltham, MA) to quantitate the cellular expression of TLR-21, IL-1β, IL-6, IFNγ, IL-4, IL10 and TGF-β. Briefly, each reaction involved a pre-incubation period of 50° C. for 2 min followed by 95° C. for 2 min, followed by 40-50 cycles of 95° C. for 10 s, 55-64° C. for 5 s, depending on the primers binding suitability, and the elongation step was 72° C. for 10 s. Subsequent melt curve analysis was performed by heating to 95° C. for 15 s, cooling to 60° C. for 1 min, and heating to 95° C. for 15 s. Primers for the amplification of all genes were synthesized by Integrated DNA Technologies (Coralville, IA), and the primer sequences are given in Table 1. Relative expression levels of all target genes were calculated relative to the reference gene β-actin.

Flow cytometry: Blood samples (2 ml) were collected from each group and diluted 1:1 with Hank's Balanced Salt Solution (HBSS). Four ml of diluted blood was carefully layered on top of Histopaque-1077 (Sigma Aldrich, St. Louis, MO) in a 15 ml conical tube and then centrifuged at 400×g for 30 min at room temperature with breaks off. Peripheral blood mononuclear cells (PBMCs) from the interphase layer were carefully aspirated into a new tube and washed twice with HBSS at 1650 rpm for 10 min at 4° C. Then the pellet was resuspended in 1 ml HBSS. Following the cell count, the suspension was adjusted to 1×10⁷/mL and 100 μL of which was used for staining. Briefly, cells were plated on 96 well round-bottom plates with each well containing 10⁶ cells in 100 μL FACS buffer (PBS with 1% BSA). Primary antibodies were added to each well (0.5-1 μg/10⁶ cells). Cells were stained in two different panels of antibody staining due to the paucity of antibody reagents available in multi-color formats (Table 2). All monoclonal anti-chicken antibodies with suggested cross-reactivity to turkey cell markers were purchased from Southern Biotech Inc. (Birmingham, AL), which were of mouse origin, and their respective clones are given the parenthesis below. The first staining panel used antibodies against CD4 (CT-4), CD8 (CT-8 recognizing CD8α chain), CD28 (AV-7), and IgM (M-1), and the second panel used anti-CD4 (CT-4), CD8 (CT-8 recognizing CD8α chain), CD44 (AV-6), and MHC-II (2G11). In both panels, cell viability dye, Live/Dead near IR (Invitrogen, CA) was used to exclude dead cells. Stained cells were washed and fixed with 4% paraformaldehyde before data acquisition using LSR-II flow cytometer (BD Biosciences). The gating strategy included exclusion of doublet cells through forward and side scatters, height and width followed by gating on live cells. Live cell gating was furthermore used as a backbone population to obtain CD4+ and CD8+ cells, CD4+CD44+ cells, CD8+CD44+ cells, CD4+CD28+ cells, CD8+CD28+ cells, and MHC-II+ cells. Single stain and fluorescence minus one control were used for fluorochrome compensation and gating positive cell populations. Data analysis was carried out using the FlowJo software version 10.8.2 (Tree Star, Ashland, OR).

ELISA: The IgY antibody titers were determined by an indirect ELISA method. Microtiter plates were coated with the purified recombinant ntATX-D1 or ntATX-D2 antigens at 2.5 g/ml in 0.1 M carbonate buffer, pH 9.6 overnight at 4° C. After the coated plates were blocked for 60 min at 37° C. with PBS containing 1% BSA (Sigma, St. Louis, MI), sera from the immunized birds were added to the wells in doubling dilutions and incubated for 2 h with coated plates at room temperature. After the plates were washed with PBS with 0.1% Tween 20 (PBST), horseradish peroxidase (HRP)-conjugated goat anti-turkey IgY (heavy and light chains; Southern Biotech, Birmingham, AL) diluted to 1:10000 in PBST, 1% BSA was added to the microplates, and the mixture was incubated for 60 min at room temperature. After five repeated washing of the plates with PBST using a Bio-Tek plate washer (Winooski, VT), the color reaction was developed by using the HRP substrate solution (Pierce TMB substrate kit, Waltham, MA), following the manufacturer's instructions. The reaction was stopped by adding 50 ul of the stop solution (0.16 M Sulfuric acid) to each well and the absorbance was measured at 450 nm using a Bio-Tek microplate reader.

Statistical analysis: All the data related reported here were analyzed using GraphPad Prism V9.5.1 (GraphPad software, San Diego, CA, USA). Data related to gross, and histopathology were analyzed by the non-parametric Kruskal-Wallis test and the values were expressed as the median score. The gene expression and cellular data were first tested for normal distribution (Shapiro-Wilk test) followed by one-way ANOVA (parametric or non-parametric) test analyses. The normally distributed data were analyzed by Tukey's multiple comparison test, while the data not normally distributed were analyzed by the Kruskal-Wallis test followed by Dunn's multiple comparison test. The asterisks above the median range or standard error of mean bars denoted in the graphs indicate statistical significance; *P<0.05, **P<0.01 or ***P<0.001.

### Results

Expression and purification of recombinant proteins: Based on the sequence-based structure function relationship of C. septicum ATX, two non-toxic (nt) regions devoid of critical sites for toxicity were identified (FIG. 1A). The regions, ntATX-D1 (912 bp) devoid of proteolytic cleavage site (Arg-367-Ser-368) and ntATX-D2 (531 bp) devoid of a signal peptide sequence (residue 1-31) and the region corresponding to cytolytic, pore-forming domain of ATX (residue 203-232) were chosen for cloning. Genes encoding ntATX-D1/ntATX-D2 were amplified from C. septicum (FIG. 1B) and cloned into the TA cloning vector (pCR2.1) followed by expression using the vector, pET14b, as histidine-tagged recombinant ntATX-D1 (˜45 kDa) or ntATX-D2 (˜25 kDa) proteins. The purified recombinant proteins were confirmed by immunoreactivity with anti-His antibodies in Western blot assays, along with the empty vectored E. coli clones as the negative control, NCx (FIG. 1C). Furthermore, serum collected from turkeys experimentally infected with C. septicum in another study (unpublished) was also used in Western blot analysis and found that both ntATX-D1 and ntATX-D2 purified proteins showed immunoreactivity, as shown in FIG. 1D. Furthermore, the blood agar plate-based hemolytic assay showed that the purified proteins were devoid of toxicity (FIG. 1E).

Protection assessment: The ability of ntATX-D1 and ntATX-D2 proteins to induce protection against an experimental C. septicum challenge in turkeys, when administered parenterally, was assessed by monitoring mortality in birds post-challenge, and evaluating gross lesions and histopathological changes in both local (skin and muscle) and systemic (spleen) tissues. Two control groups, namely unchallenged/negative control (NCx) and adjuvant-only plus challenged/positive control (PCx) were used alongside of ntATX-D1 immunized (D1) and ntATX-D2 immunized (D2) groups of birds.

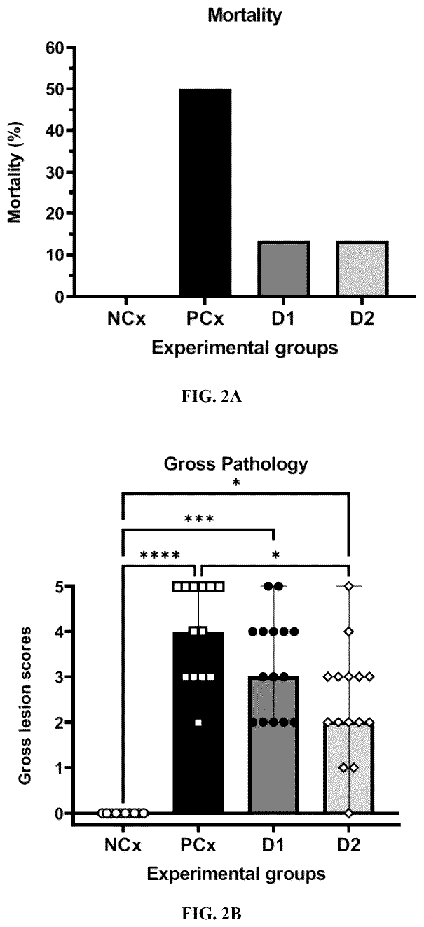

As shown in FIG. 2, a mortality of 50% was observed in PCx group, while the rate of mortality in D1 and D2—immunized groups was 13.3%. Gross pathology scores showed that all the challenged groups developed lesions when compared the uninfected NCx birds, while the D2—immunized turkeys had significantly reduced lesions than the PCx groups, indicating protection against experimental CD infection (FIG. 2). It was noteworthy that the total number of birds with lesions scored ≤2 in the D2—immunized group was 53.3% (8 of 15), when compared to PCx group that had 7% (1 out of 13), indicating that the D2-immunized birds were able to resist the progression of CD severity. Although the D1-immunization resulted in 33.33% (5 out of 15) of birds with an average score of ≤2, this group had 7 birds that developed severe lesions, scored ≥4, supporting a lack of statistically significant protection against CD in this group.

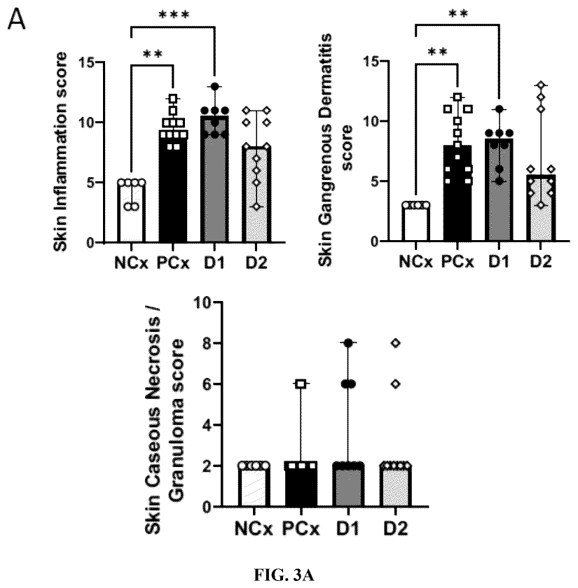

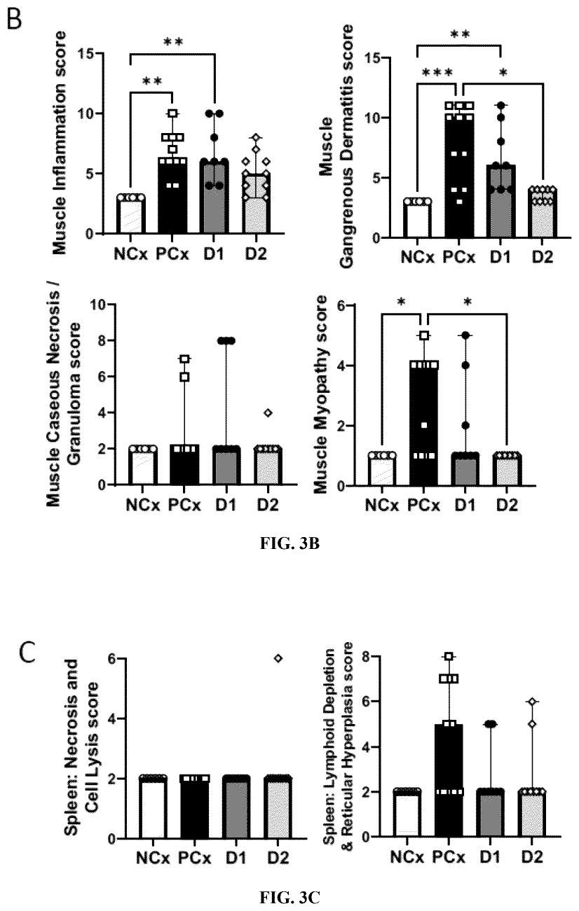

As shown in FIG. 3, the histopathological changes in the skin and muscle tissues were evaluated based on the criteria of inflammation, gangrenous dermatitis and caseous necrosis/granuloma lesions, while the splenic changes were evaluated based on the necrosis and cell lysis, and splenic lymphoid depletion and hyperplasia. The evaluation of skin tissues showed that the PCx and D1-immunized groups had significantly higher scores for lesions characterized under the categories of inflammation and gangrenous dermatitis, when compared to NCx group (FIG. 3A). However, no significant difference was observed between the D2 and NCx groups. No significant changes were observed in the skin caseous necrosis/granuloma lesion category between the groups. The muscle tissue examination revealed that the PCx group had significantly higher scores of lesions indicative of inflammation, gangrenous dermatitis and myopathy, when compared to NCx birds (FIG. 3B). Additionally, the D2-immunized birds also had significantly higher scores related to inflammation and gangrenous dermatitis lesions in comparison to NCx group of birds. However, the scores of lesions characteristic of gangrenous dermatitis and myopathy in the D2-immunized birds were significantly lower in comparison to those in the PCx group. No significant changes were observed in the muscle caseous necrosis/granuloma lesion category between the groups. Furthermore, no significant changes were observed in the splenic necrosis and cell lysis, or splenic lymphoid depletion and hyperplasia between the groups (FIG. 3C). However, the PCx group had a numerically increased score for the lesions characterized by splenic lymphoid depletion and hyperplasia.

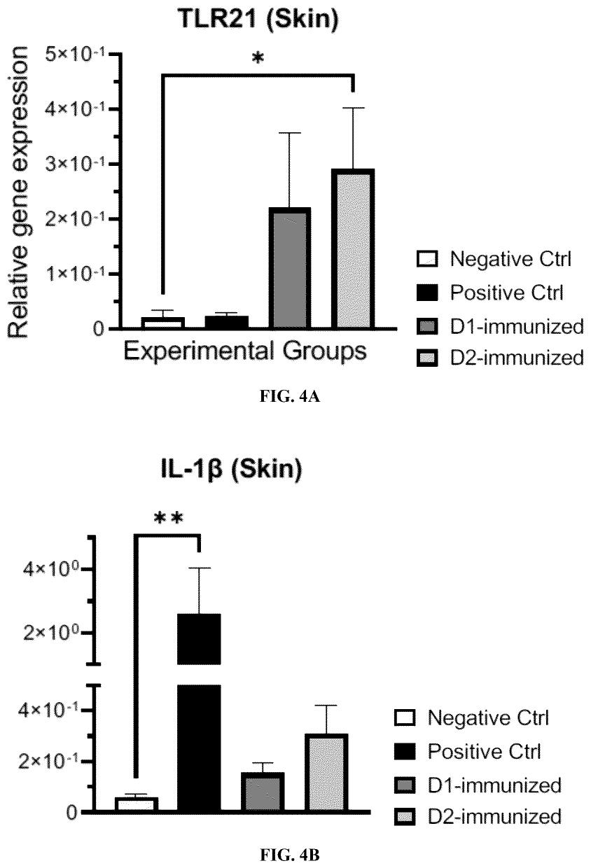

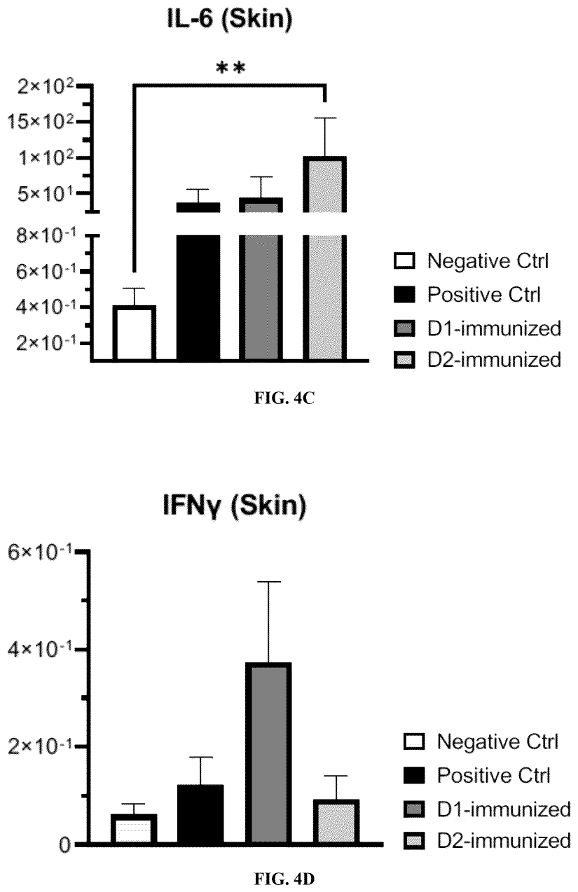

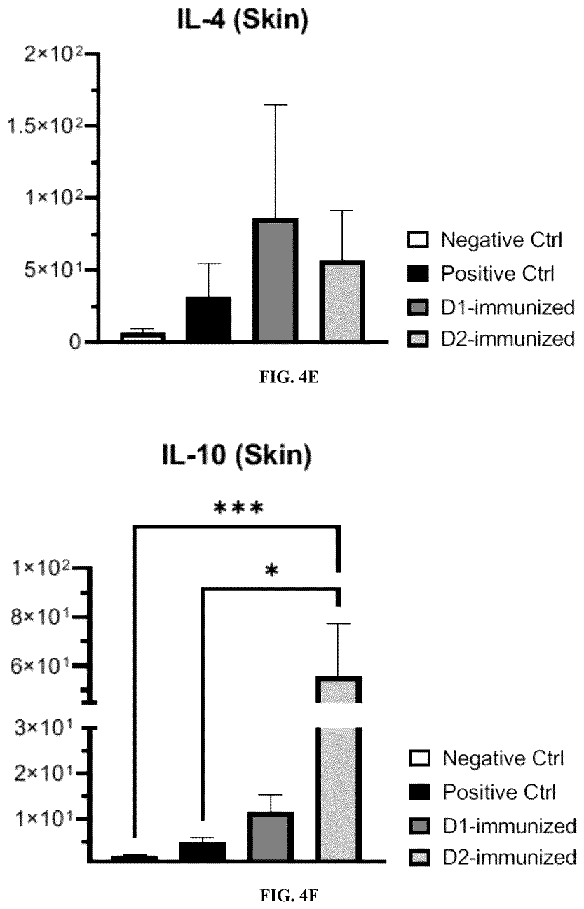

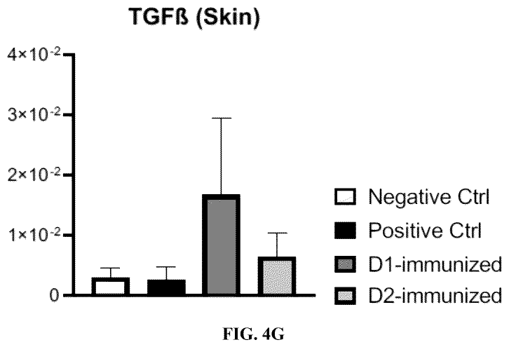

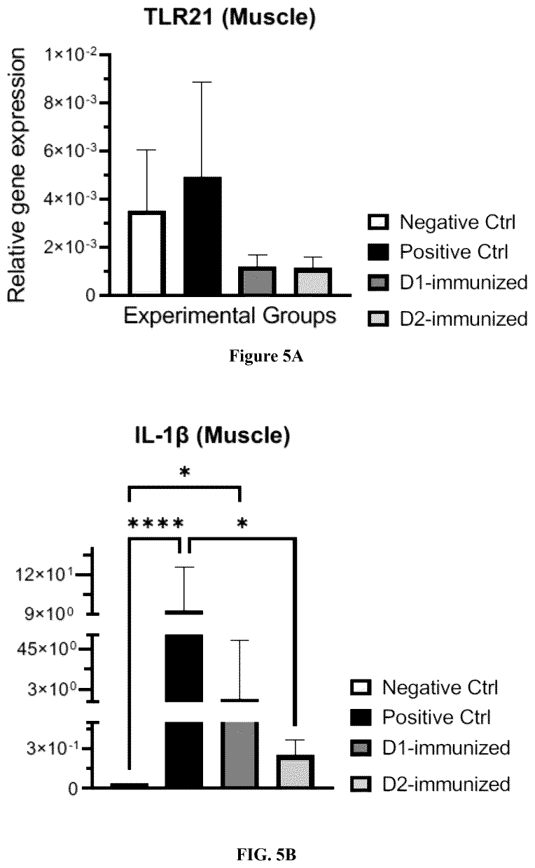

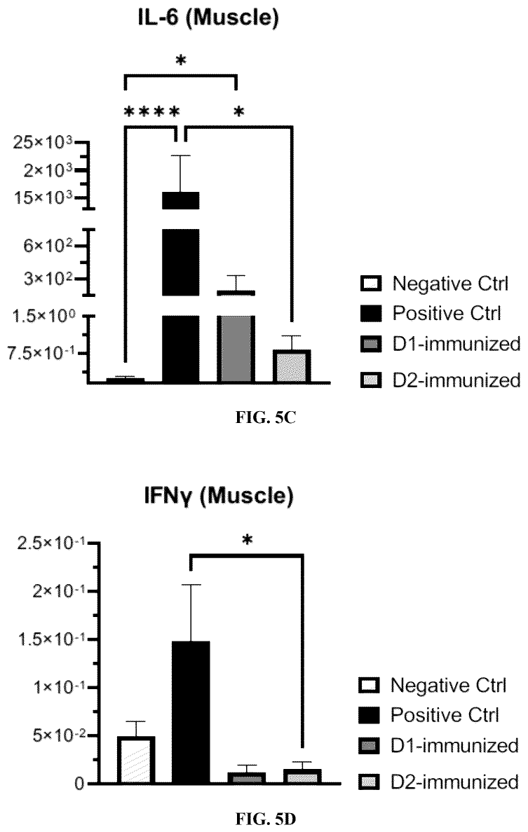

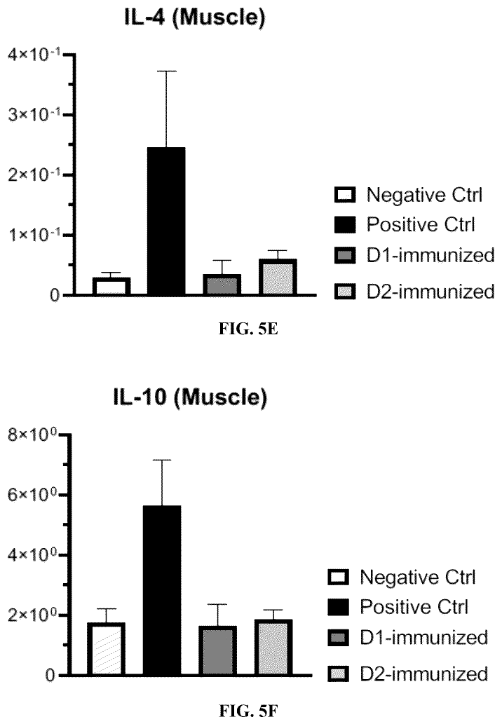

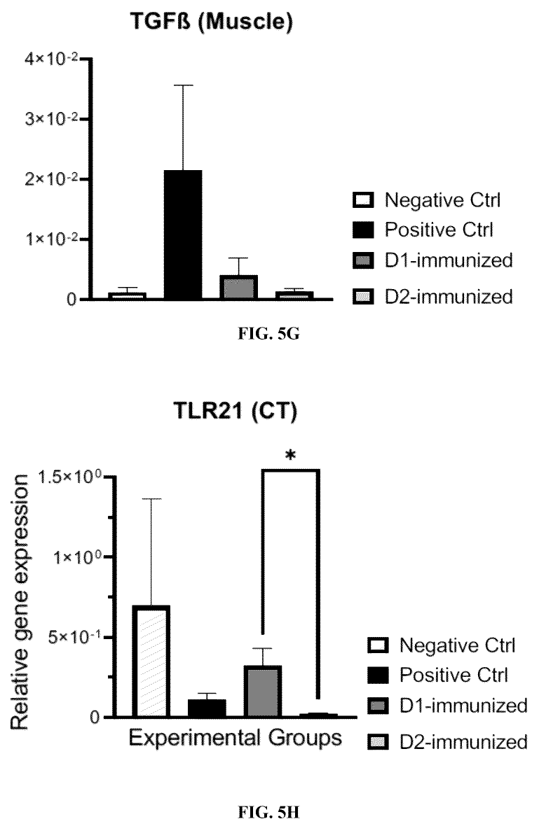

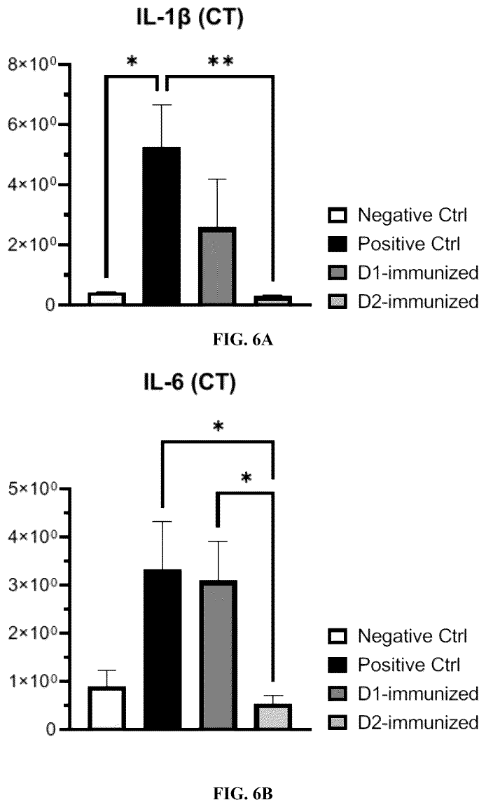

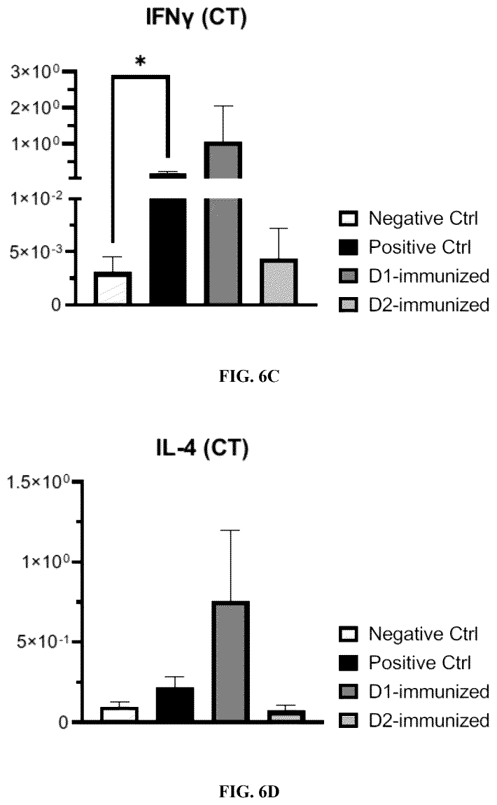

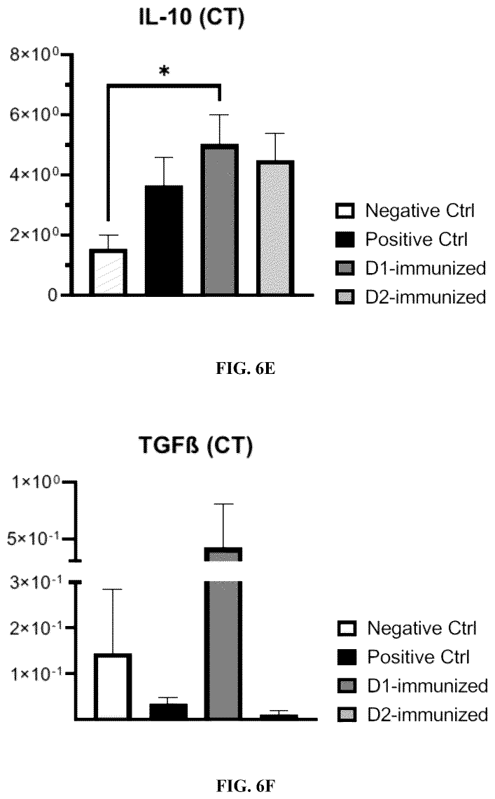

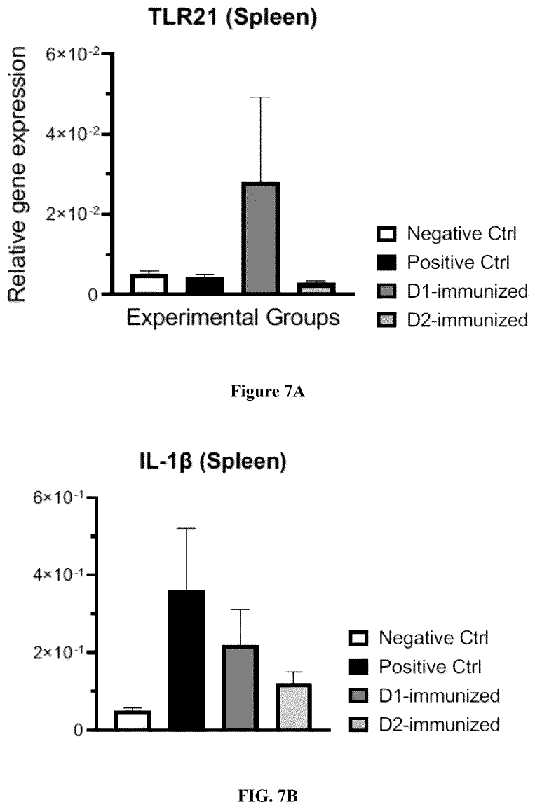

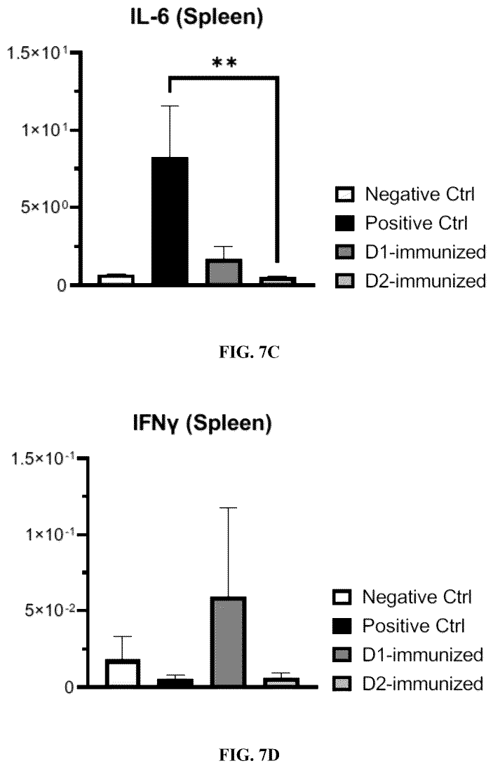

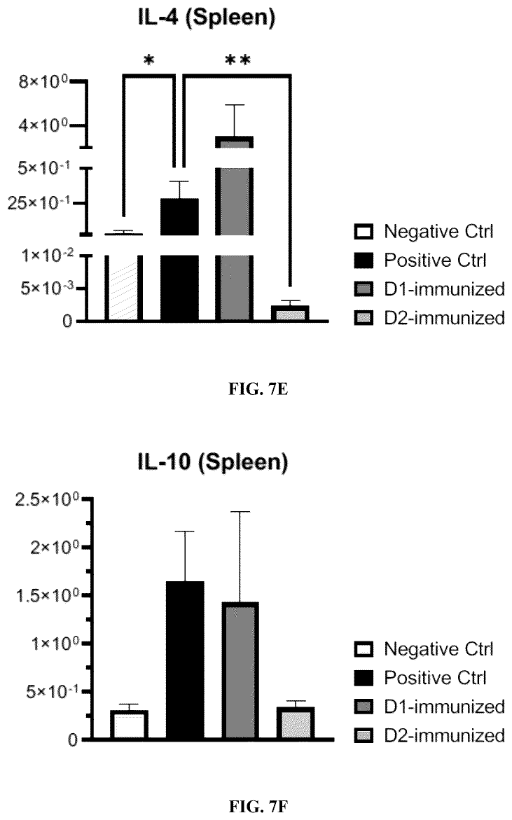

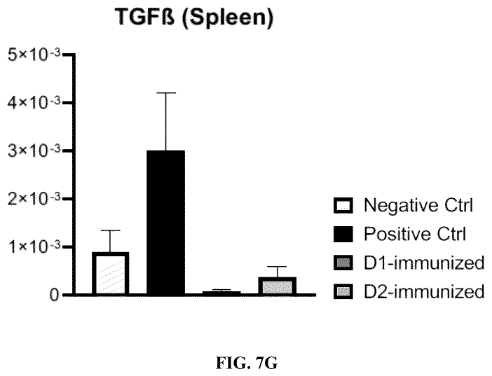

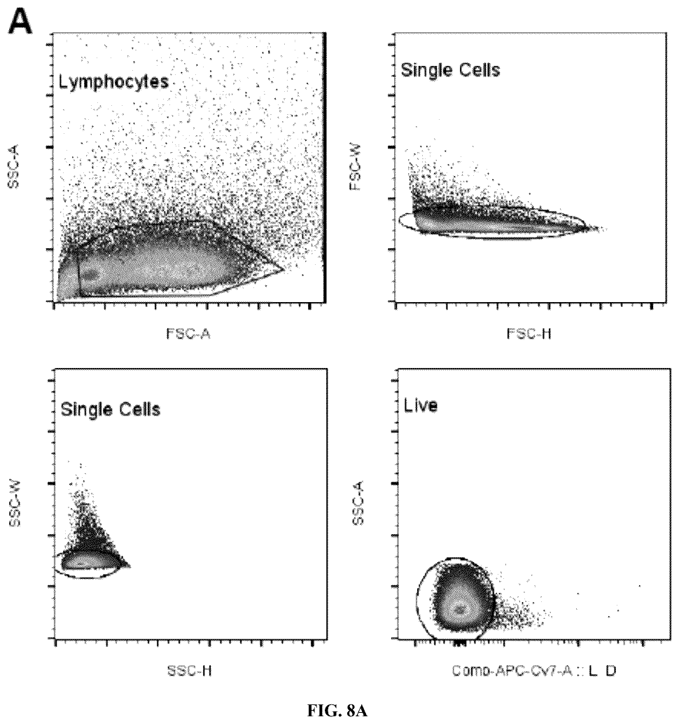

Immune gene expression: Expression of TLR-21, IL-1B, IL-6, IFNγ, IL-4, IL10 and TGF-β genes in the skin, muscle, cecal tonsil and spleen tissues from the D1 and D2-immunized groups along with the NCx and PCx controls was determined. In the skin (FIG. 4), transcription of IL-1β in the PCx birds was found significantly increased when compared to NCx group, while D2-immunized birds bad significantly higher expression of IL-6 and IL-10 genes when compared to NCx birds. Additionally, the IL-10 transcription in D2-immunized birds was also significantly higher than those in the PCx group. Furthermore, D2-immunized birds had significantly increased TLR21 gene expression when compared to NCx birds. However, no significant changes in the expression of IFNγ, IL-4 or TGF-β genes between groups was observed. In the muscle (FIG. 5), the expression of IL-1β and IL-6 genes in the PCx and DI groups was significantly increased when compared to NCx group, while the D2-immunized birds had significantly downregulated expression of these genes (IL-1β and IL-6) than the PCx birds. Furthermore, the transcription of IFNγ in the D2-immunized group was also significantly downregulated than the PCx group. No significant changes were observed in the expression of TLR21, IL-4, IL-10 or TGF-β genes between the groups. In CT (FIG. 6), the transcription of IL-1β and IFNγ genes in PCx was significantly higher compared to NCx, while the expression of IL-1β and IL-6 genes in D2-immunized birds was significantly downregulated than PCx. Moreover, expression of IL-6 and TLR21 genes in D1-immunized group was significantly upregulated compared to D2-immunized birds. Additionally, the transcription of IL-10 gene was significantly higher in D1-immunized birds than the NCx group. However, no significant changes observed in the expression of IL-4 and TGFβ genes between the groups. In spleen (FIG. 7), IL-4 transcription in PCx group was significantly higher than the NCx group, while the expression of IL-6 and IL-4 genes in the D2 immunized birds was significantly downregulated compared to PCx group. No significant changes observed in the transcription of IL-1β, IFNγ TLR21, IL-10 or TGFβ genes between the treatment groups.

In addition to the figures presented here, a summary highlighting the statistically significant (P<0.05) changes the immune gene expression in positive control (PCx), D1-immunized and D2-immunized groups in comparison to the unchallenged negative control (NCx) is given in Table 3.

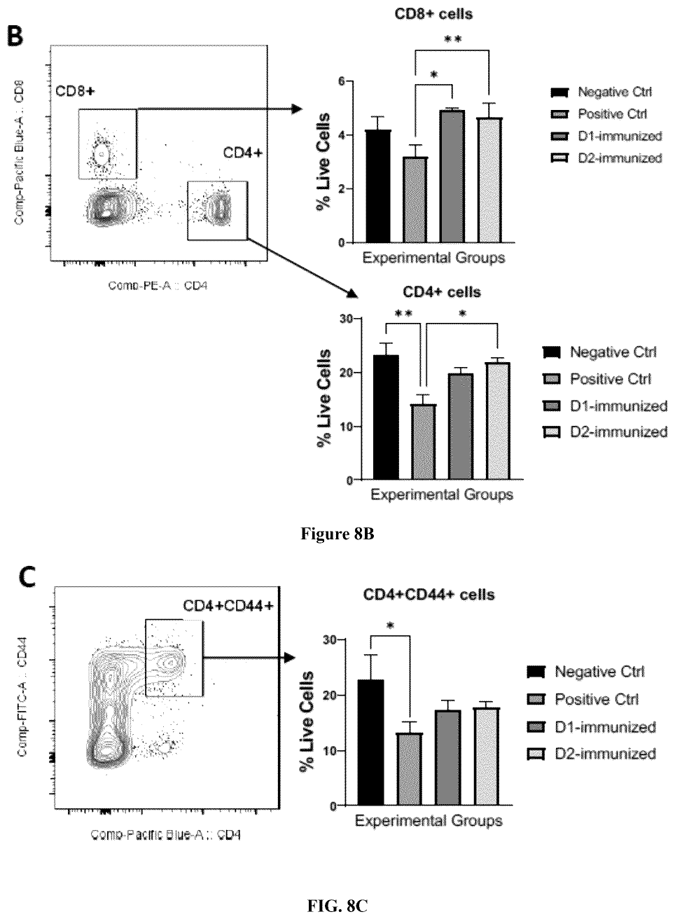

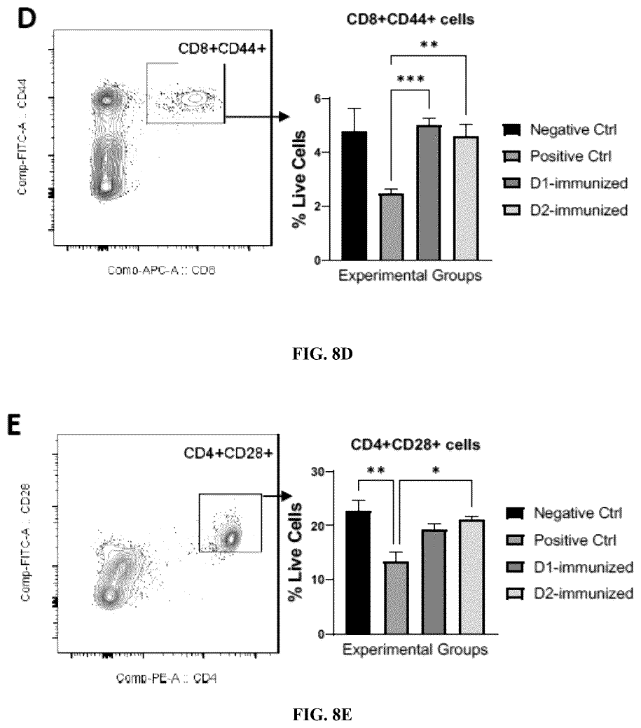

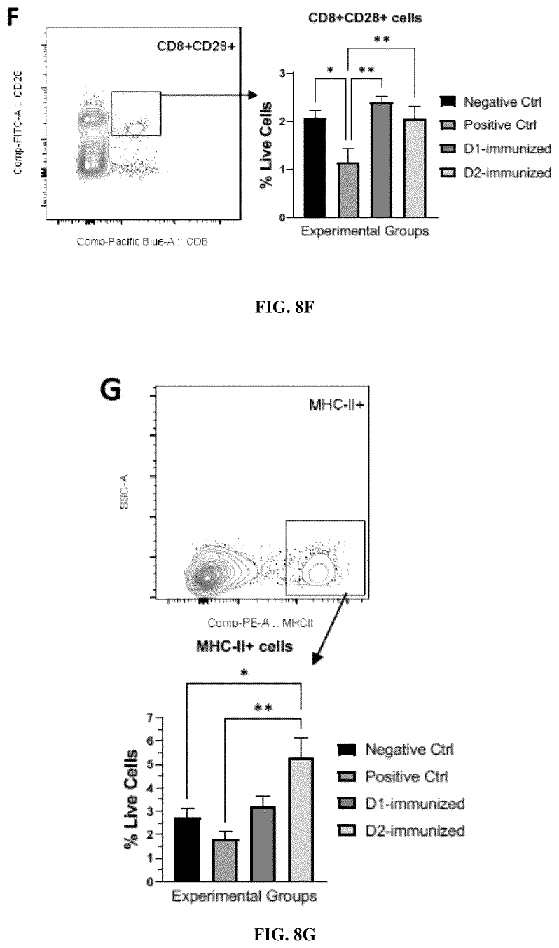

Immunophenotyping: To evaluate the immunization-induced cellular responses, the frequencies of CD4+, CD4+CD28+ and CD4+CD44+, CD8+, CD8+CD28+ and CD8+CD44+, as well as MHC-II+ cell populations in the PBMCs were analyzed using flow cytometry. Due to the unavailability of turkey-specific antibody reagents for use in flow cytometry staining, a pilot study was conducted to identify anti-chicken antibodies with reported cross-reactivity to turkey cell markers as well as to titrate optimal antibody binding (data not shown). Although the anti-IgM antibody was included in one of the two staining panels, its binding to turkey cells in obtaining a distinct positive cell population was unsuccessful; hence, it was excluded from the present study analysis. The gating strategy used to allow an accurate analysis of these cell types is shown in FIG. 8A. It is noteworthy that the lack of anti-chicken CD3 cross-reactivity with turkey CD3 led us to using the ‘live’ cells as the backbone population for gating the CD4+/CD8+ cells. The frequency of CD4+ cells (FIG. 8B), CD4+CD44+ cells (FIG. 8C) and CD4+CD28+ cells (FIG. 8E) were significantly decreased in PCx birds compared to NCx group. However, the D2-immunized birds were found to have significantly increased populations of CD4+ cells (FIG. 8B), and CD4+CD28+ cells (FIG. 8E) when compared to PCx group. No significant changes in the CD4+ cells (FIG. 8B) and CD4+CD44+ (FIG. 8C) frequencies were observed in D1 and D2 group when compared with the NCx group. The frequency of CD8+ cells (FIG. 8B), CD8+CD44+ cells (FIG. 8D) and CD8+CD28+ cells (FIG. 8F) cells were significantly increased in D1 and D2 immunized birds than the PCx group. Additionally, the birds in the PCx group were found to have significantly decreased populations of CD8+CD28+ cells (FIG. 8F) in comparison to those in the NCx group. Furthermore, the frequency of MHC-II+ cells was significantly higher in D2-immunized group when compared to the NCx and PCx groups.

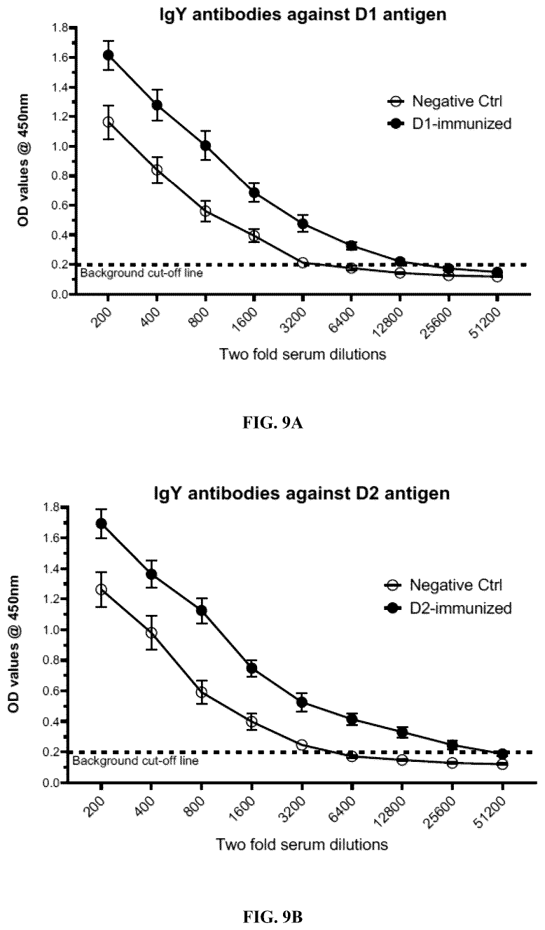

Antibody evaluation: The sera collected, prior to the C. septicum challenge, from immunized turkeys along with negative control were evaluated to determine antigen (ntATX-D1 or ntATX-D2)-specific IgY levels. As shown in FIG. 9, the immunized birds showed IgY-mediated seroconversion to the ntATX-D1 or ntATX-D2 antigen, as determined by their OD values over the course of 9-point serum doubling dilutions.

Discussion: Clostridial dermatitis, caused by the Clostridial pathogens, specifically C. septicum being often implicated in commercial outbreaks, has posed a major economic challenge to the poultry industry, particularly the turkey sector. The economic losses due to CD include high mortality and necrotic lesions in the skin and muscle, leading to carcass condemnations. Although the therapeutic antibiotics can control CD, their availability and the effectiveness coupled with the increasing risk of antimicrobial resistance warrant immediate antibiotic alternative strategies such as vaccines. To this end, C. septicum alpha toxin (ATX) is the focus herein to identify two non-toxic domains, namely the ntATX-D1 and ntATX-D2 and develop subunit protein-based vaccines for immunizing turkeys against CD using a virulent C. septicum challenge model. The results found that while both proteins were able to effectively prevent mortality, the ntATX-D2 provided a more robust protective immunity, as determined by gross and histopathology findings as well as immunological parameters.

Clostridium septicum is a Gram-positive, anaerobic, spore forming and toxin-producing bacterium with ATX being the most cytolytic necrotizing toxin, implicated as the key virulence factor in the CD pathogenesis. It was reported that C. septicum induces a robust inflammatory response in local as well as systemic tissues during CD in turkeys, and much of it is attributable to the ATX-mediated damage. Herein, it was found that the ntATX domains, specifically the ntATX-D2, can significantly prevent mortality and reduce CD-induced pathology shows that ATX is a key virulence determinant and that vaccines exploring the immunogenic and protective potential of this toxin can yield effective CD control solution. Furthermore, the ntATX-D1 and ntATX-D2 immunizations leading to an antigen-specific seroconversion indicates that antibodies may play an important role in protection against CD in turkeys. Previous work using the C. septicum bacterin toxoid for immunizing turkeys against CD have implied the importance of antibodies against ATX in alleviating CD-induced mortality and disease severity. For example, previous work evaluating the safety and efficacy of a C. septicum bacterin-toxoid vaccine in commercial turkeys found that birds vaccinated subcutaneously showed no adverse reactions and developed bacterin-specific serum antibodies (See, Thachil AJ, McComb B, Kromm M, Nagaraja KV. Vaccination of turkeys with Clostridium septicum bacterin-toxoid: evaluation of protection against clostridial dermatitis. Avian diseases. 2013;57(2):214-9. Epub 2014 Apr. 3. doi: 10.1637/10421-101512-Reg.1. PubMed PMID: 24689176). Additionally, the vaccinated farms were found to have a reduced morbidity and mortality, showing that toxoid-based vaccination strategy can offer a viable platform for CD control in turkeys. More recently, another study showed that adjuvantation of C. septicum bacterin-toxoid with a water-in-oil Montanide emulsion administered turkeys subcutaneously in commercial farm setting can reduce CD morbidity and mortality along with increased serum antibodies. Furthermore, a study by Lancto et.al., (2014) evaluated the efficacy of a noncytolytic C. septicum alpha-toxin (NCAT) peptide as a potential vaccine candidate in turkeys against CD when administered with or without C. septicum bacterin+alpha-toxoid. The NCAT was constructed by excluding the 28-aminoacid cytolytic domain from the native ATX, for use as a vaccine when administered subcutaneously. Results found that while the NCAT immunization was able to reduce mortality, the birds receiving bacterin+alpha-toxoid showed the highest protective efficacy when compared to other groups. Although these studies infer that ATX offers protective immunogenicity, the findings herein provide specific information related to the ATX domains, specifically the ntATX-D2 region, that play a key role in CD immunity. It was also noteworthy that the clinical protection observed in the ntATX-D2 immunized groups was not only supported by the reduced mortality but also by the significant reduction gross and histopathology lesion scores. A case in point was the histopathological observation that ntATX-D2 immunized birds had significantly reduced muscle lesions associated with gangrenous dermatitis and myopathy compared to the unimmunized (PCx) group, while no inflammatory changes were observed in the skin of these birds. These findings showed that ntATX-D2 offers superior ability over ntATX-D1 in inducing protective immunity in turkeys, and thus, can be an efficacious vaccine candidate for use in vaccine research applications.

The present disclosure not only demonstrates the protective effect of ATX but also provides insights into the underlying protection mechanisms related to immune gene expression and T-cell expression, including CD4 and CD8. These findings highlight the role of ATX in protective immunogenicity and the mechanisms by which it functions are highly significant.

### Example 2: Recombinant Lactococcus lactis-Based Oral Vaccine Expressing Non-Toxic Alpha Toxin Domains of Clostridium septicum Can Offer Protection Against Experimental Clostridial Dermatitis in Turkeys

Clostridial dermatitis (CD), caused by the anaerobic spore-forming Clostridium septicum bacteria, is an important emerging disease of turkeys. Despite its economic burden on the poultry industry, there are no efficacious vaccines currently available for CD control in turkeys. Two non-toxic domains of C. septicum alpha toxin (ATX), namely ntATX-D1 and ntATX-D2, were identified and showed that subcutaneous immunization of turkeys with purified recombinant subunit ntATX proteins can offer protection against CD. In the present study, the pNZ8124-NICE vector®-based Lactococcus lactis (Str. NZ9000) cloning system was used to express ntATX-D1 and ntATX-D2 proteins, and immunized turkeys orally at 7, 8 and 9 weeks of age followed by a virulent C. septicum challenge at one-week post-last immunization. Results showed that while both ntATX-D1 and ntATX-D2 vectored-L. lactis vaccines could effectively prevent mortality, the ntATX-D2 carrying vaccine conferred significantly stronger protective immunity, as determined by gross and histopathological evaluations. Additionally, the immunized birds were found to have antigen-specific serum IgY antibodies. Furthermore, the L. lactis-ntATX-D2 vaccinated+C. septicum-challenged turkeys had significantly reduced expression of pro-inflammatory cytokine (Interleukin-1β, IL-6 and Interferon-γ) genes in the skin, muscle, spleen and cecal tonsil tissues when compared to unvaccinated+challenged group. These findings show that a L. lactis-based oral recombinant probiotic vaccine expressing ntATX-D2 of C. septicum alpha-toxin provides protective immunity against CD in turkeys.

Introduction: Clostridial dermatitis (CD), also known as gangrenous dermatitis or cellulitis, is an emerging economically devastating disease of poultry, specifically the turkeys. It is reported that CD affects about 40-50% of turkey grower farms in the United States causing economic losses due to poor production rates and sudden spikes in mortality. Clinically, CD in turkeys usually peaks around 13-18 weeks of age and is characterized by necrotic dermatitis with edema and/or emphysema in the underlying subcutaneous tissues and sudden death. The two most known Clostridial pathogens causing CD are C. septicum and C. perfringens, while C. septicum has more often been isolated and identified as the primary etiological agent for CD in turkeys. Several managemental and environmental predisposing factors are also implicated in the development of CD in turkeys, which include overcrowding, poor ventilation, inadequate hygiene, built-in/deep litter production system and immunosuppression. Although the therapeutic antibiotics can control CD, prompt availability of effective antibiotics coupled with increased risks associated with the spread of antimicrobial resistance (AMR) warrant an urgent need for alternatives to antibiotics, such as vaccines.

The CD pathogenesis is proposed to involve two theories based on the host entry of C. septicum, which are Gram-positive, anaerobic, spore-forming and toxin-producing bacteria with alpha-toxin (ATX) implicated as the pathogen's key virulence factor. The “Outside-in” theory suggests pathogen's entry through skin culminating in tissue damage and septicemia, while the “Inside-out” theory suggests the pathogen entering from the intestine into circulation and eventually reaching the skin to cause dermatitis. In support of the former theory, it has been previously shown that turkeys inoculated with C. septicum subcutaneously can cause clinical CD, characterized by typical dermatitis lesions and related mortality. Irrespective of these proposed theories, the C. septicum ATX, which is a cytolytic, pore-forming, lethal and necrotizing toxin has been the focus of developing CD vaccines. It has recently been shown that C. septicum infection in turkeys, involves a robust inflammatory immune response, both locally and systemically, and much of the tissue damage seem to have been attributed ATX. Antibodies against ATX have been shown, by us and others, to reduce morbidity and mortality in turkey affected with CD. Considering the critical role of ATX in CD pathogenesis and immunity, a previous study by Lancto et al. (2014) has shown that subcutaneous immunization of turkeys with a noncytolytic C. septicum alpha-toxin (NCAT) peptide devoid of the 28-amino acid cytolytic domain from the native ATX can provide partial protection against CD. More recently, two non-toxic domains of C. septicum ATX, referred to as ntATX-D1 and ntATX-D2, were identified and cloned and expressed in Escherichia coli as recombinant subunit proteins to immunize turkeys subcutaneously. The findings showed that immunization with ntATX proteins, specifically the ntATX-D2, can significantly protect turkeys against CD, suggesting ntATX-D1 and ntATX-D2 can be promising vaccine candidates.

Considering the parenteral routes for vaccine delivery being unsuitable for commercial poultry applications, the present study involved developing Lactococcus lactis (food-grade probiotic)-based oral vaccines expressing ntATX-D1 and ntATX-D2. Turkeys were administered orally with recombinant vectored vaccines followed by assessing the immunogenicity and protective efficacy using an experimental C. septicum infection-induced CD model. The clinical and immune parameters assessed for protection evaluation included (1) Mortality and Gross pathology, (2) Histopathological examination of skin, skeletal muscle, and spleen tissues, (3) Immune gene expression in skin, muscle, spleen, and cecal tonsil (CT) tissues, and (4) Serology.

Overall, the study demonstrated that the oral administration of L. lactis-based vaccines expressing ntATX-D1 and ntATX-D2 elicits strong immune responses in turkeys and provides significant protection against CD, highlighting that they are effective and practical vaccines for commercial poultry applications.

### Materials and Methods

Bacterial Strains and Plasmids: Clostridium septicum Str. B1, previously isolated from commercial turkeys affected with CD, served as the focal strain for this investigation. Cultures of C. septicum were grown anaerobically in Reinforced Clostridial medium (Oxoid, Lenexa, KS) at 37° C. for 36-48 hours. Nontoxic domains of Alpha toxin (ntATX) were amplified from C. septicum str. B1 DNA using primers detailed in Table 1. Following amplification, the genes were incorporated into the pCR 2.1 TA cloning vector (Invitrogen, Carlsbad, CA) and subsequently introduced into TOP10′ E. coli competent cells. These cells were then cultivated at 37° C. in Luria-Bertani (LB) broth/agar supplemented with the appropriate antibiotic and 40 μl of 40 mg/ml X-Gal. The ntATX domains were further cloned into the pNZ8124 NICE vector (MoBiTec) designed for protein secretion, utilizing the signal sequence of the L. lactis major secreted protein Usp45. The resulting constructs were transformed into the rec A+ E. coli strain MC106 and cultured in LB broth/agar supplemented with 10 g/ml chloramphenicol. Additionally, the recombinant pNZ8124 (rpNZ8124) was introduced into Lactococcus lactis subsp. cremoris strain NZ9000, a derivative of L. lactis subsp. cremoris MG1363, which harbors the regulatory genes nisR and nisk integrated into the pepN gene of MG1363. Cultures of transformed cells were grown in M17 broth (Gibco, CA) and maintained anaerobically at 30° C. without agitation, facilitated by a gas pack system.

Cloning and Nisin Induction of Gene expression in Lactococcus lactis: Based on the sequence information of ATX two regions were identified and predicted to be non-toxic when expressed as recombinant proteins. The initial region, termed non-toxic ATX Domain1 (ntATX-D1), a 912 bp segment and lacks the crucial proteolytic cleavage site (Arg-367-Ser-368), necessary for the toxin's activity. The second region identified as non-toxic ATX Domain 2 (ntATX-D2) consists of 531 bp segment devoid of both the signal peptide sequence (residues 1-31) and the domain responsible for cytolytic pore-formation (residues 203-232) in ATX. The PCR condition included 35 cycles of amplification with each cycle consisting of steps of denaturation at 94° C. for 1minute, annealing at 55° C. for 1 minute, extension at 72° C. for 1 minute and a final extension at 72° C. for 7 min. Subsequently, the amplified gene segments were initially ligated between the XhoI and BamHI restriction enzyme sites into the pCR 2.1 TA cloning vector (Invitrogen, Carlsbad, CA) and transformed into TOP10′ E. coli competent cells. After Sanger sequencing, the D1 and D2 domains were cloned into the pNZ8124 (MoBiTec), NICE vector for protein secretion with the signal sequence of the lactococcal major secreted protein Usp45, and then transformed into the rec A+ E. coli strain MC106. L. lactis NZ9000 glycerol stocks were prepared for electroporation after culturing in G/L-SGM17B medium (M17 Broth with 0.5M sucrose, 2.5% glycine and 0.5% glucose) for 2 days. Transformation of L.lactis NZ9000 cells was conducted using the rpNZ8124 plasmid via electroporation, using 0.2 cm Gene Pulser Electroporation Cuvettes (Bio-Rad), following the manufacturer's recommendations. Parameters included 2000V, 25 μF, and 200Ω, resulting in a pulse duration of 4.5-5 msec. After electroporation, cells were incubated on ice for 5 minutes following the addition of 1 ml of G/L-SGM17B medium supplemented with 20 mM MgCl2 and 2 mM CaCl2, then subsequently incubated for 1-1.5 hours at 30° C. Transformed cells were then plated onto M17 agar with glucose (G+M17) and 10 μg/mL of chloramphenicol, and colonies were allowed to grow for 48 hours at 30° C. Confirmation of transformants was carried out through colony PCR, plasmid DNA isolation and Sanger sequencing.

Transformed L. lactis were passaged for a couple of days to ensure that the bacteria were stable in the presence of antibiotic (10 μg/ml Chloramphenicol). 1/25^{th} of the overnight culture were added to fresh medium and grown until the OD600 ˜ 0.5. Induced the culture with 7-9 ng/ml nisin and incubated for another 20-24 hrs. Then collected cells by centrifugation and resuspended cells in sterile water and cell free extract was concentrated using Amicon Ultra-15 Centrifugal filter units (Sigma-Aldrich) and were tested for protein production by SDS-PAGE. Immunoreactivity of the recombinant ntATX-D1 and ntATX-D2 proteins in the supernatant was confirmed by Western Blot analysis. The primary serum from turkeys affected by CD, collected in a previous study, was used with an antibody dilution of 1:200 followed by HRP-conjugated IgM antibody at a 1:200 dilution was used in immunoreactivity analysis.

To confirm the transcription, ntATX-D1/D2+pNZ8124+L. lactis was inoculated in G+M17 media and incubated anaerobically overnight at 37° C. Cells were harvested by centrifugation and stored at −80° C. Pelleted cells were treated with TRIzol® and homogenized with 0.1 mm glass beads at high speed for 5 minutes, with intervals of one minute on ice between cycles. Cell debris was removed by centrifugation, and the supernatant was transferred to an RNase-free tube. Total RNA was extracted and subjected to DNase I treatment following the manufacturer's protocol (Zymo Research). Subsequently, cDNA synthesis was performed using 500-1000 ng of purified RNA with a High-Capacity RNA-to-cDNA kit (Applied Biosystems, Waltham, MA), according to the manufacturer's instructions. The resulting cDNA was used as a template for PCR with specific internal primers to assess the transcription of ntATX-D1/D2 in L. lactis.

For protein expression, transformed L. lactis were passaged for two days to ensure that the bacteria were stable in the presence of antibiotic (10 ug/ml chloramphenicol), diluted to 1:25 of the overnight culture to fresh medium (30° C.) and grown to OD 600˜0.5. Cultures were induced with 7-9 ng/ml nisin and incubated for another 20-24 hr. Then cells were collected and resuspended in sterile water and cell free extract was concentrated using Amicon Ultra-15 Centrifugal filter units (Sigma-Aldrich). Protein expression was assessed by SDS-PAGE and immunoreactivity of ntATX-D1 and ntATX-D2 was confirmed by Western Blot using primary serum from turkeys affected by CD, collected in a previous study, and followed by HRP-conjugated IgM antibody at 1:200 dilutions for immunoreactivity analysis.

Immunization and Challenge: The animal experimental procedures utilized in this investigation were granted approval by the Institutional Animal Care and Use Committee of North Carolina State University (IACUC protocol 22-181-A). Six-week-old male turkeys were obtained from Butterball Farms LLC. (Goldsboro, NC) and housed in the Laboratory Animal Research (Biosafety Level 2) facility of the College of Veterinary Medicine, North Carolina State University. They were provided with fresh wood shavings as litter, along with ad libitum access to water and non-medicated grower feed. Each bird was individually identified with leg bands and allocated into four experimental groups, including two control groups, with one group subjected to C. septicum challenge and the other not. The negative control (NCx) group (n=10) consisted of birds that did not receive the C. septicum challenge. However, this group included five birds that were orally immunized with L. lactis+pNZ8124+ntATX-D1, and five birds received L. lactis+pNZ8124+ntATX-D2. The positive control (PCx) group (n=11) received pNZ814+Lactococcus lactis in G+M17 media and were subsequently challenged with C. septicum. The experimental groups, termed ‘D1’ (n=13) and ‘D2’ (n=12), immunized orally with L. lactis+pNZ8124+ntATX-D1 or ntATX-D2 protein, respectively, followed by challenge with C. septicum. The birds in the NCx group were housed in isolation from the other groups, whereas the PCx, D1, and D2 groups shared a common room, albeit with segregation facilitated by vacant floor pens. The NCx group served as the control to assess the impact of the C. septicum challenge on the other groups and to identify any potential adverse effects associated with immunization. Concurrently, the PCx group served as a control to quantify the protective efficacy of the administered immunizations.

The birds underwent a vaccination regimen, at week 7, 8 and 9 of age, consisting of three doses in total. Initially, each bird received a 3 ml (1×10⁹ CFU/mL) of oral immunization containing a mixture of L. lactis+pNZ8124+ntATX-D1 or ntATX-D2 protein, in G+M17 media. For the second dose, the volume administered was increased to 4 ml per bird. Finally, the third and last dose was given at a volume of 5 ml per bird. This progressive increase in dose volume was aimed to potentially enhance the immune response and efficacy of the vaccination protocol.

For the experimental infection/challenge at one week following the last immunization, C. septicum Strain B1, isolated from commercial turkeys afflicted with Clostridial dermatitis were employed in a previous study. C. septicum cultures were cultivated anaerobically at 37° C. for 36-48 hours in Reinforced Clostridial medium (Oxoid, Lenexa, KS). Each bird in the infected group received a subcutaneous dose of 0.5×10⁸ CFU/mL in the lower pectoral region, with 0.75 mL administered on each side, while the unchallenged group of birds were administered growth medium only. Birds were monitored for clinical signs and mortality for a period of 72 hours, and gross lesions were scored during necropsy or at euthanasia for those that died prior to study termination. Samples (skin, skeletal muscle, and spleen) were collected for histopathological analysis, while for immune gene expression analysis, the skin, skeletal muscle, spleen, and cecal tonsil tissues were collected.

To recover L. lactis from the intestines of immunized birds, the contents from ileum and ceca were collected at necropsy. The contents were serially diluted in 1×PBS, and the diluted samples were plated on M17 supplemented with 0.5% Glucose and 10 ug of Chloramphenicol to obtain individual colonies of L. lactis. The agar plates were then anaerobically incubated overnight at 30° C. To confirm the identity of isolated colonies as L. lactis, 16S rRNA PCR was performed using specific L. lactis 16S primers (Table 1). PCR conditions were as follows: an initial cycle at 94° C./3 min, 55° C./45 s, 70° C./1 min; followed by 30 cycles of 94° C./45 s, 55° C./45 s, 70° C./1 min; and a final cycle at 94° C./45 s, 55° C./45 s, 70° C./5 min. PCR products were examined by electrophoresis on a 1% agarose gel. Subsequently, to recover the vaccine strain of L. lactis from the intestine, colony PCRs were performed using ntATX-D1 and ntATX-D2 specific primers.

Pathology: The gross lesions were evaluated based on a scoring system developed previously. The lesions were assessed in the skin, muscle, and spleen. Lesions were graded as follows: 0 for no observable lesions, 1 for the presence of dark red/purple-green discoloration and blisters on the skin, 2 for cellulitis (Grade 1) characterized by moist or weepy skin with subcutaneous edema, localized in smaller areas (1-3 cm) exhibiting exudative hemorrhagic foci, 3 for cellulitis (Grade 2) characterized by larger areas (>3 cm) with exudate accumulation, hemorrhagic regions, and gas-filled (crepitus) areas, 4 for subcutaneous lesions extending to muscle, showing pale red or tan discoloration, multifocal to coalescing hemorrhage, and necrosis, and 5 for septicemia, indicating systemic organ involvement with spleen, liver, heart, or other organs displaying organomegaly along with extensive multifocal tan foci and hemorrhage.

Histopathological examination was conducted on skin, skeletal muscle, and spleen samples obtained from birds exhibiting grossly apparent symptoms, both during the clinical monitoring period and upon necropsy at the conclusion of the study. Tissues were fixed in 10% neutral buffered formalin for a minimum of 24 hours, followed by processing, sectioning at 5 um thickness, and staining with hematoxylin and eosin. Subsequently, sections were analyzed by board-certified veterinary pathologists for lesions, with blinded scoring. Additionally, select sections were subjected to Gram staining to detect Gram-positive Clostridium rods. In the evaluation of skin and skeletal muscle, criteria such as inflammation (including fibrin or edema, heterophils, and lymphocytic or histocytic lesions), gangrenous dermatitis (comprising fibrin or edema, cell lysis, and bacterial presence), and caseous necrosis or granuloma formation were considered. Splenic changes were assessed based on parameters including necrosis, cell lysis, and alterations in splenic lymphoid depletion and hyperplasia. Lesions were scored on a scale ranging from 0 to 5, with 0 indicating the absence of lesions, 1 representing focal lesions affecting less than 5% of the tissue section, 2 denoting multifocal lesions scattered across 5-25% of the tissue, 3 indicating extensive multifocal lesions involving 30-70% of the tissue, and 5 indicating diffuse lesions affecting more than 75% of the tissue. Photomicrographs representing histopathological lesions in the skin, muscle and spleen are shown in FIG. 23, FIG. 24 and FIG. 25, respectively.

Quantitative real-time PCR: Skin, muscle, spleen, and Cecal Tonsil samples were collected from turkeys across all experimental groups (n=8-10) and preserved in RNAlater solution (Invitrogen, Carlsbad, CA) before being stored at −80° C. until further processing. Total RNA extraction was carried out using a Bead Ruptor Elite Bead Mill Homogenizer (OMNI International, Kennesaw, GA) with 1.4 mm Ceramic Beads (OMNI International, Kennesaw, GA) in TRIzol® reagent (Invitrogen, Carlsbad, CA), following the manufacturer's protocol. The extracted RNA was then treated with a DNA-free Kit (Invitrogen, Carlsbad, CA). Subsequently, cDNA synthesis was conducted using a High-Capacity RNA-to-cDNA kit (Applied Biosystems, Waltham, MA) with 500-1000 ng of purified RNA, following the manufacturer's instructions. The resulting cDNA was diluted at a 1:10 ratio in nuclease-free water for subsequent real-time PCR analysis. Quantitative real-time PCR employing SYBR Green was carried out on the diluted cDNA using a QuantStudio 6 Flex System and QuantStudio Real-Time PCR Software (Applied Biosystems, Waltham, MA) to measure the cellular expression levels of TLR21, IL-1β, IL-6, IL-4, and IL-10. Each PCR reaction included a pre-incubation period of 2 min at 50° C. followed by 2 min at 95° C. Cycling conditions comprised 50-55 cycles of denaturation at 95° C. for 10 s, annealing at 55-64° C. for 5 s (depending on primer binding suitability), and extension at 72° C. for 10 s. Subsequently, melt curve analysis was performed by heating to 95° C. for 15 s, cooling to 60° C. for 1 min, and reheating to 95° C. for 15 s. Primers for amplifying the target genes were synthesized by Integrated DNA Technologies (Coralville, IA), and the primer sequences are provided in Table 1. Relative expression levels of all target genes were calculated in reference to the β-actin gene.

ELISA: The IgY antibody titers were determined by an indirect ELISA assay. Microtiter plates were coated with the purified recombinant ntATX-D1 or ntATX-D2 antigens at 5 μg/mL in 0.1 M carbonate buffer, pH 9.6 overnight at 4° C. After the coated plates were blocked for 60 min at 37° C. with PBS containing 1% BSA (Sigma, St. Louis, MI), sera (n=8) from the immunized birds were added to the wells in doubling dilutions starting at 1:200 dilution of the whole sera samples and incubated for 2 h with coated plates at room temperature. After the plates were washed with PBS with 0.1% Tween 20 (PBST), horseradish peroxidase (HRP)-conjugated goat anti-turkey IgY (heavy and light chains; Southern Biotech, Birmingham, AL) diluted to 1:10000 in PBST, 1% BSA was added to the microplates, and the mixture was incubated for 60 min at room temperature. After five repeated washing of the plates with PBST using a Bio-Tek plate washer (Winooski, VT), the color reaction was developed by using the HRP substrate solution (Pierce TMB substrate kit, Waltham, MA), following the manufacturer's instructions. The reaction was stopped by adding 50 μl of the stop solution (0.16 M Sulfuric acid) to each well and the absorbance was measured at an optical density (OD) of 450 nm using a Bio-Tek microplate reader.

Statistical analysis: All data were analyzed using GraphPad Prism V9.5.1 (GraphPad Software, San Diego, CA, USA). Mortality data was analyzed by simple survival analysis (Kaplan-Meier) followed by Mantel-Cox as well as Gehan-Breslow-Wilcoxon tests. Gross pathology and histopathology data were assessed using the non-parametric Kruskal-Wallis test, with results presented as median scores. Gene expression and cellular data were initially checked for normal distribution using the Shapiro-Wilk test, followed by one-way ANOVA (parametric or non-parametric) analyses. Normally distributed data underwent Tukey's multiple comparison test, while non-normally distributed data were analyzed using the Kruskal-Wallis test followed by Dunn's multiple comparison test. ELISA data were analyzed by comparing the average OD values of immunized and control samples at each dilution using two-way ANOVA, applying Sidak's pairwise multiple comparison test. Statistical significance was denoted by asterisks above the median range or standard error of mean bars in the graphs: *P<0.05, **P<0.01, or ***P<0.001.

### Results

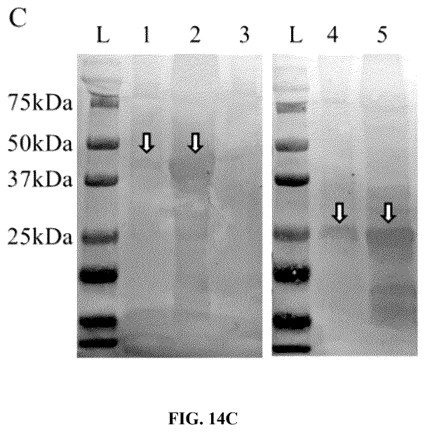

Cloning and expression of ntATX-D1 and ntATX-D2 in L. lactis: Based on sequence-structure-function analysis, two non-toxic domains within the C. septicum alpha-toxin (ATX) have been identified. The domains, designated as ntATX-DI (residues 1-304) and ntATX-D2 (residues 235-412), were selected for cloning due to the absence of critical sites for toxicity. Specifically, ntATX-D1 lacks a proteolytic cleavage site, while ntATX-D2 is devoid of both a signal peptide sequence and the cytolytic, pore-forming region of ATX. Genes encoding these domains were amplified from C. septicum and initially cloned into the pCR2.1-TA cloning vector. Subsequent expression was achieved using the pNZ8124 vector, which incorporates the Usp45 signal sequence for cell surface expression. The recombinant pNZ8124 vectors were transformed into L. lactis NZ9000 (FIG. 14A). Surface expression of ntATX-D1 (˜45 kDa) and ntATX-D2 (˜25 kDa) on L. lactis was confirmed via SDS-PAGE analysis. The concentrated recombinant proteins' immunoreactivity was confirmed by Western blot assays, using dialyzed and concentrated ntATX-D1/D2 as positive control and empty pNZ8124 in L.lactis NZ9000 as the negative control. Both the ntATX D1 and ntATX D2 supernatants showed immunoreactivity, as shown in FIG. 14C.

Transcription of ntATX-D1/D2: RNA extracted from the transformed L. lactis cells was reverse transcribed into cDNA. PCR was subsequently performed using specific primers to verify the transcription of the ntATX domains. The presence of both nontoxic domains, D1 and D2, was confirmed by their appearance on the agarose gel following PCR (FIG. 14B), indicating successful transcription of both domains.

Intestinal Colonization and Recovery of vaccine strain of L. lactis from the birds' gut: Isolated colonies of Lactococcus lactis from intestinal content were observed on the selective agar plate. To confirm the strain of these colonies, 16S rRNA PCR was performed, producing a 348 bp amplicon on the agarose gel, which confirmed the strain as L. lactis (FIG. 22C). Additionally, colony PCR was conducted on these isolated L. lactis colonies to recover the vaccine strain. Both domains, ntATX-D1 (FIG. 22A) and ntATX-D2 (FIG. 22B), were successfully amplified from these strains.

Efficacy of Lactococcus lactis (food-grade probiotic)-based oral vaccines against Clostridium septicum challenge: The protective effects of ntATX-D1 and ntATX-D2 proteins against an experimental C. septicum challenge in turkeys were evaluated in several ways. Mortality and clinical signs in birds were monitored post-challenge. Additionally, gross lesions and histopathological changes were examined in both local tissues (skin and muscle) and systemic tissues (spleen). The study included two control groups: an unchallenged negative control (NCx), a positive control (PCx), which received pNZ814 (empty vector without insert)+Lactococcus lactis in G+M17 media and were subsequently challenged with C. septicum. Alongside these controls, groups of birds immunized orally with L. lactis+pNZ8124+ntATX-D1 or ntATX-D2 protein, followed by challenge with C. septicum were also assessed.

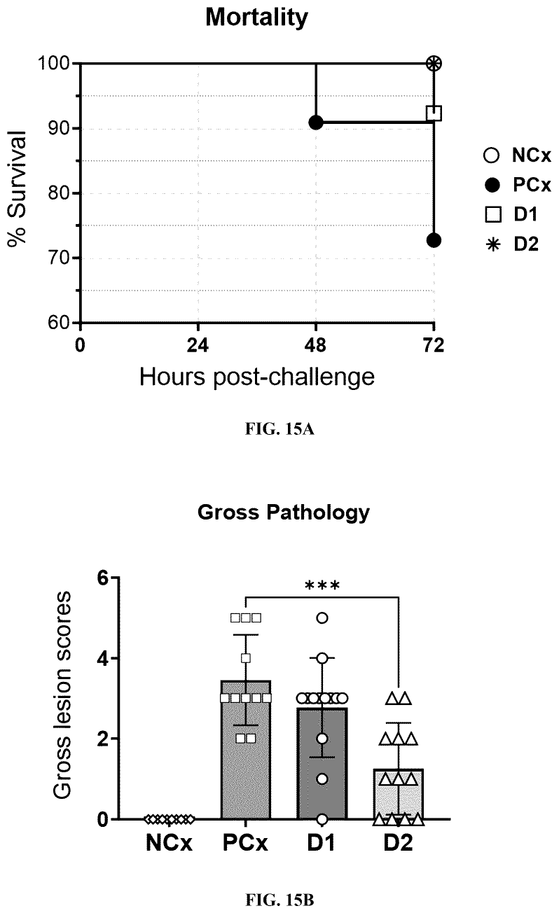

As illustrated in FIG. 15, the PCx group exhibited a mortality rate of 27% (3 out of 11 birds). In contrast, the D1-immunized group showed a delayed mortality onset, with the total mortality significantly reduced to 7.7% (1 out of 13 birds) by the end of the 72-hour observation period. Notably, the D2-immunized birds demonstrated complete protection against the C. septicum challenge, with no mortalities reported in this group throughout the study. Gross pathology scores indicated that all challenged groups developed lesions compared to the uninfected NCx birds. However, the D2-immunized turkeys exhibited significantly reduced lesion severity than the PCx group, suggesting effective protection against experimental CD infection (FIG. 15). Above all, none of the birds in the D2-immunized group had lesion scores>3, and 42% (5 out of 12) had a score of 0. In contrast, 82% (9 out of 11) of the PCx group exhibited lesion score≥3, highlighting the reduced progression of CD severity in the D2-immunized birds. The D1-immunized group, however, had 10 birds with severe lesions (scores≥3), indicating a lack of statistically significant protection against CD in this group.

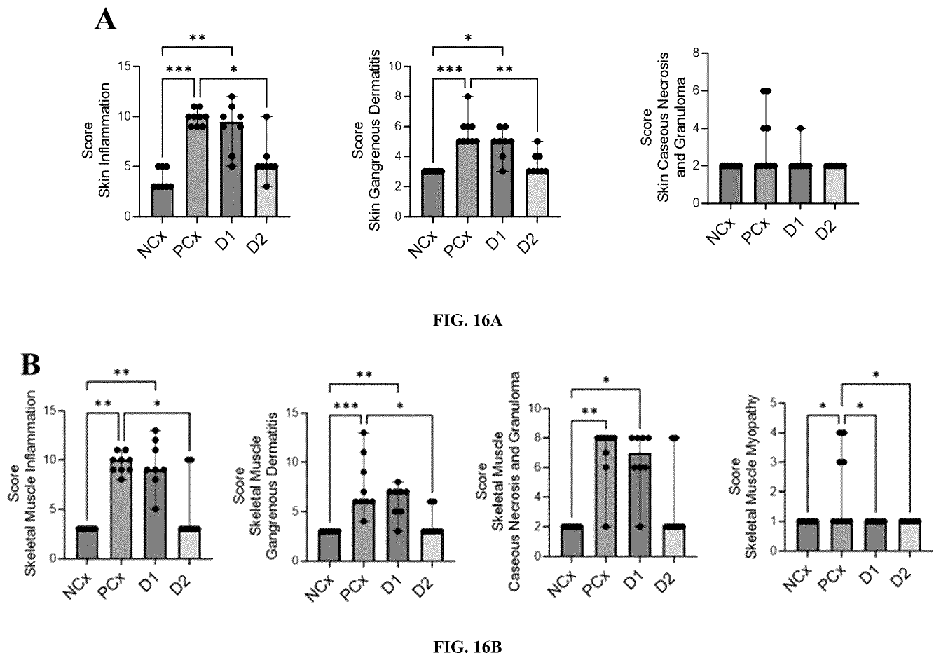

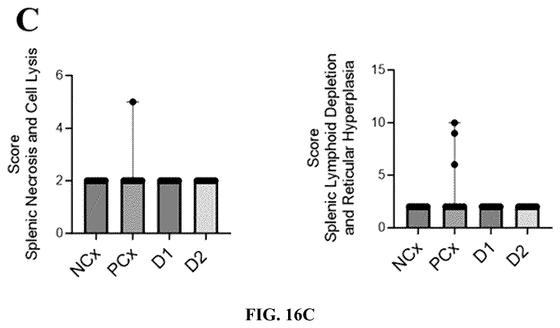

As shown in FIG. 16, histopathological alterations in skin tissues were assessed based on criteria including inflammation, gangrenous dermatitis, and caseous necrosis/granuloma lesions. Muscle tissues were evaluated using the criteria applied to skin tissues as well as myopathy. Splenic changes were analyzed according to the presence of necrosis, cell lysis, lymphoid depletion and hyperplasia. The PCx and D1-immunized groups exhibited significantly higher scores for skin lesions, particularly in the categories of inflammation and gangrenous dermatitis, compared to the NCx group (FIG. 16A). In contrast, the D2-immunized birds demonstrated significantly lower scores compared to the PCx group, with no significant differences observed between the D2 and NCx groups. Furthermore, there were no significant changes observed in the skin caseous necrosis and granuloma lesion categories across all groups. In skeletal muscle tissue, the PCx group exhibited significantly higher lesion scores indicative of inflammation, gangrenous dermatitis, caseous necrosis/granuloma, and myopathy compared to the NCx birds (FIG. 16B). Similarly, the D1-immunized birds showed significantly increased lesion scores for inflammation, gangrenous dermatitis, and caseous necrosis/granuloma relative to the NCx group. Conversely, the D2-immunized birds demonstrated significantly lower lesion scores across all categories except caseous necrosis/granuloma compared to the PCx group. Additionally, the D1 group exhibited significantly lower lesion scores in muscle myopathy compared to the PCx group. In spleen tissue, no significant differences were observed in necrosis, cell lysis, or lymphoid depletion and hyperplasia between the groups (FIG. 16C). However, the PCx group exhibited numerically higher scores for lesions characterized by necrosis and cell lysis, as well as lymphoid depletion and hyperplasia.

Immune Gene Expression: The expression levels of TLR21, IL-1β, IL-6, IL-4, and IL-10 genes were assessed in the skin, muscle, spleen, and cecal tonsil tissues from both D1-and D2-immunized groups, as well as from NCx and PCx control groups.

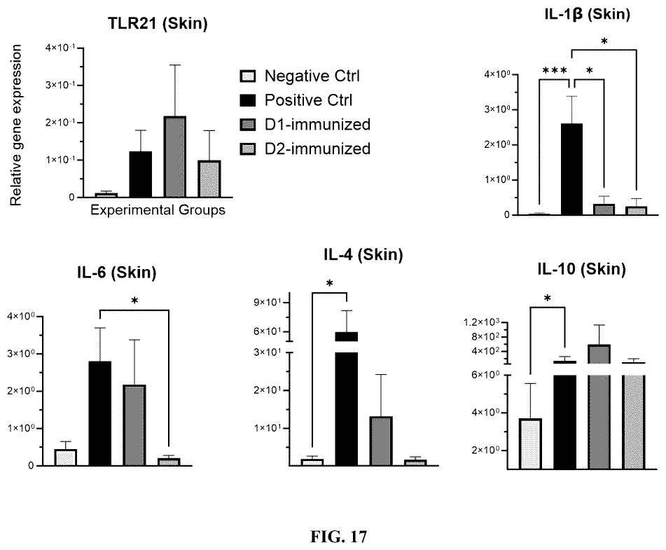

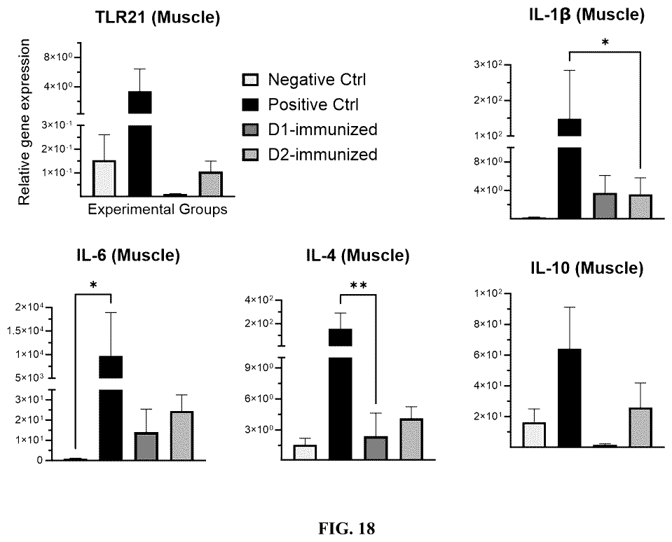

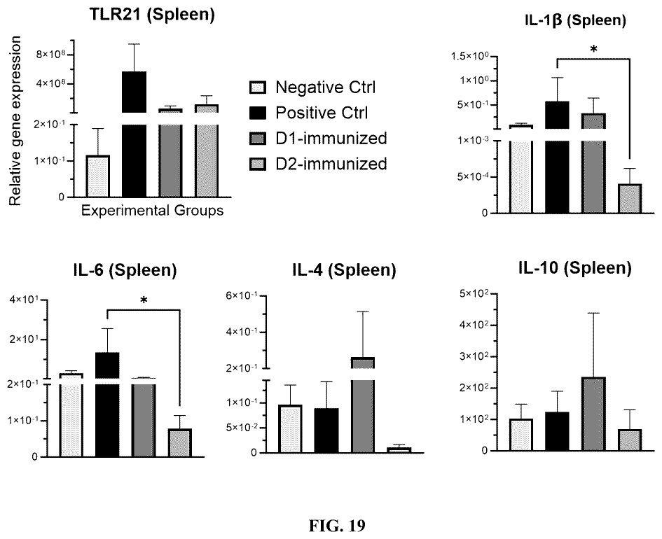

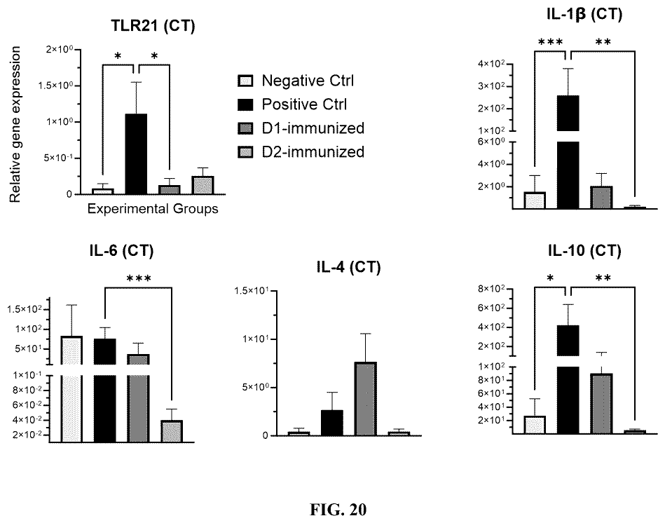

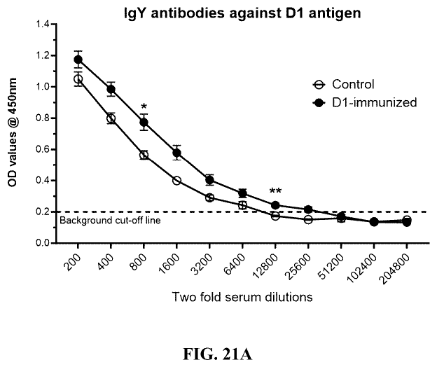

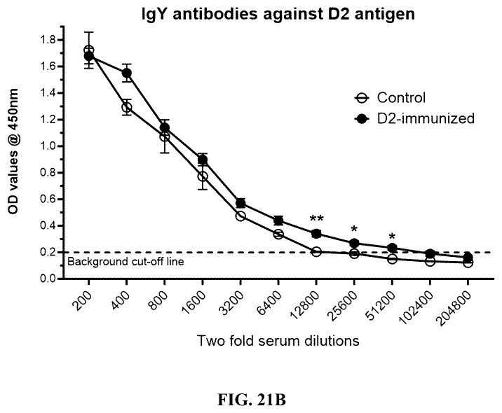

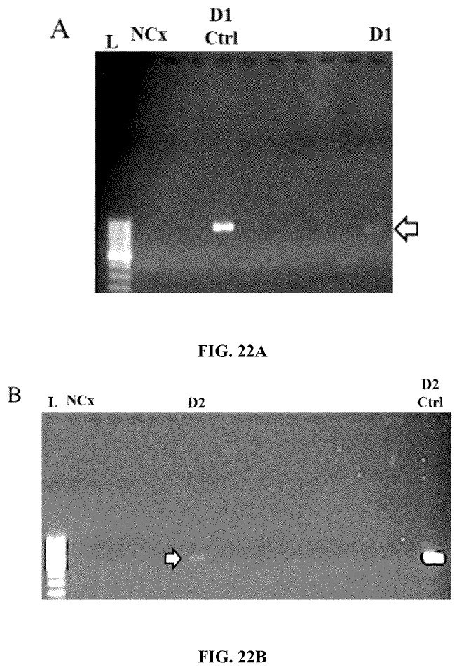

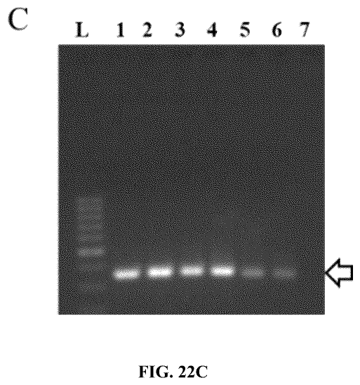

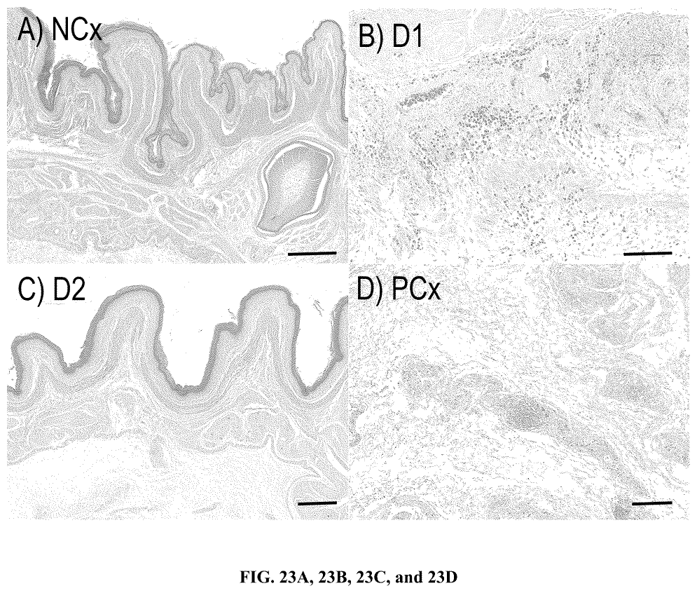

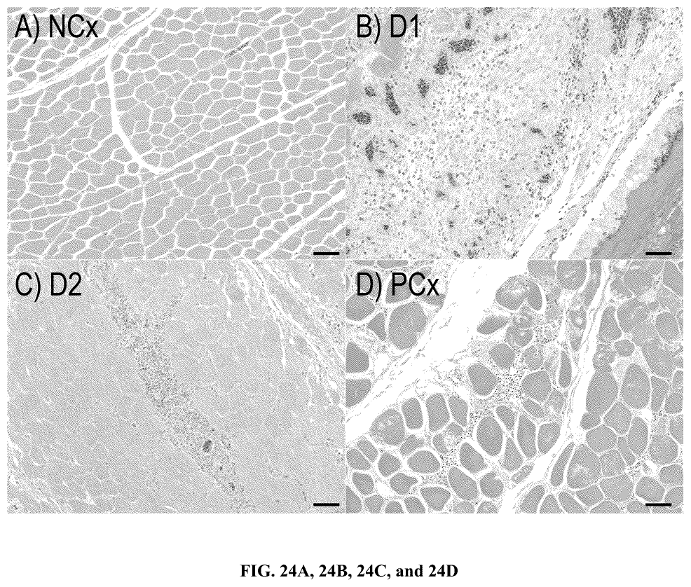

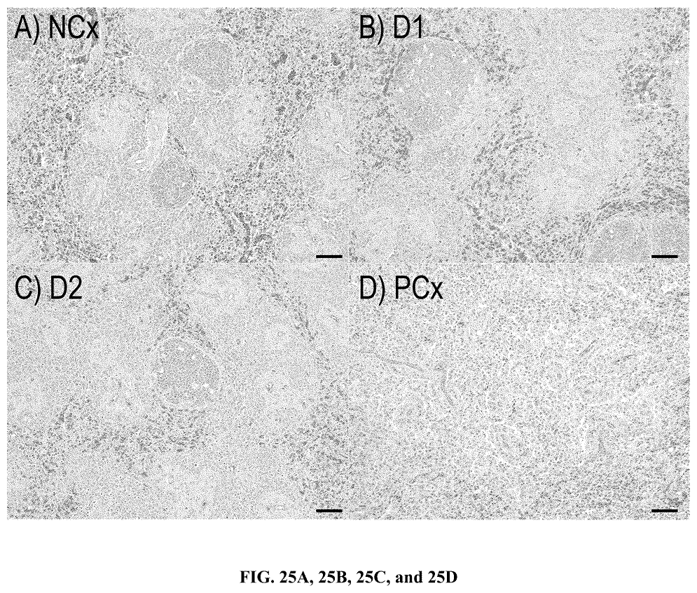

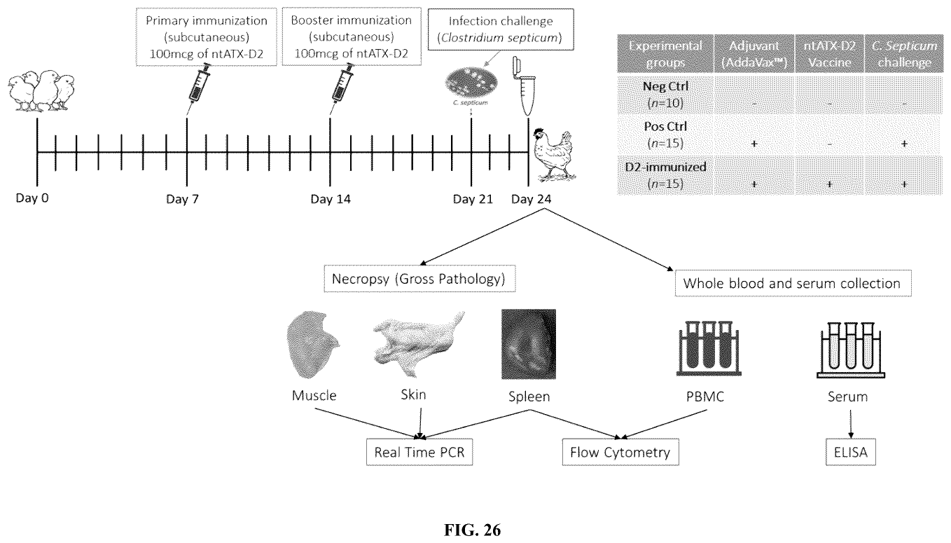

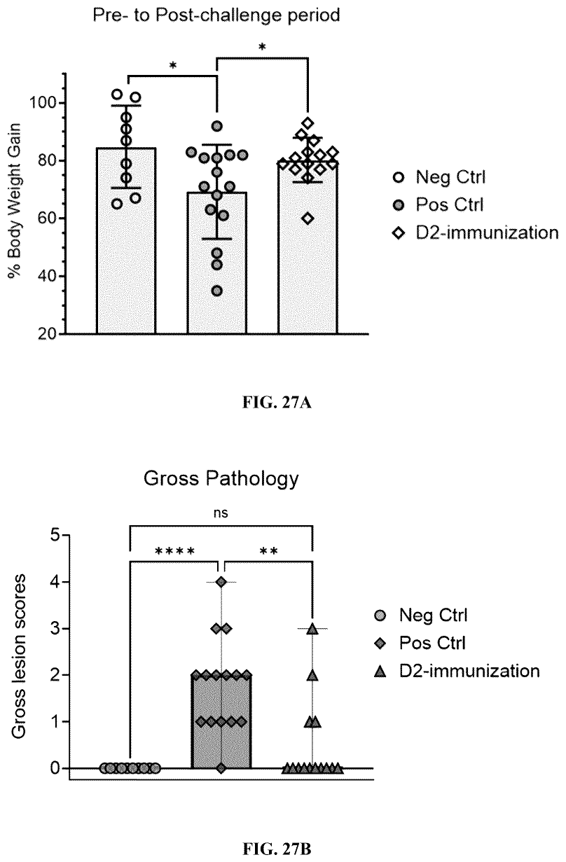

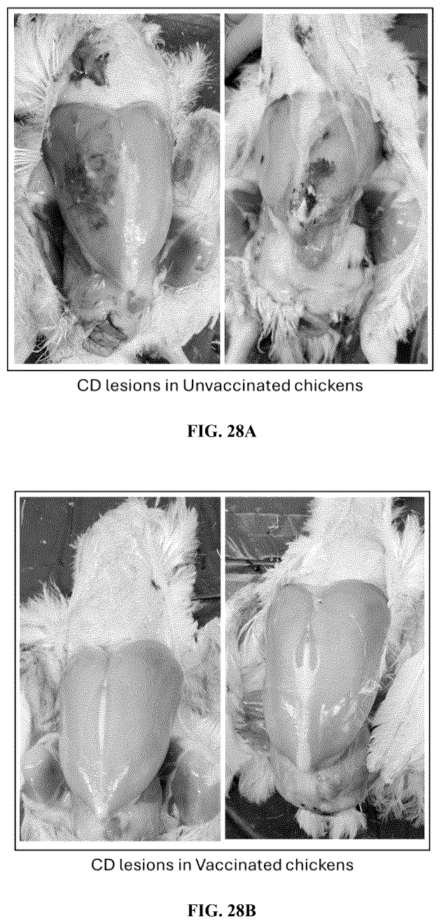

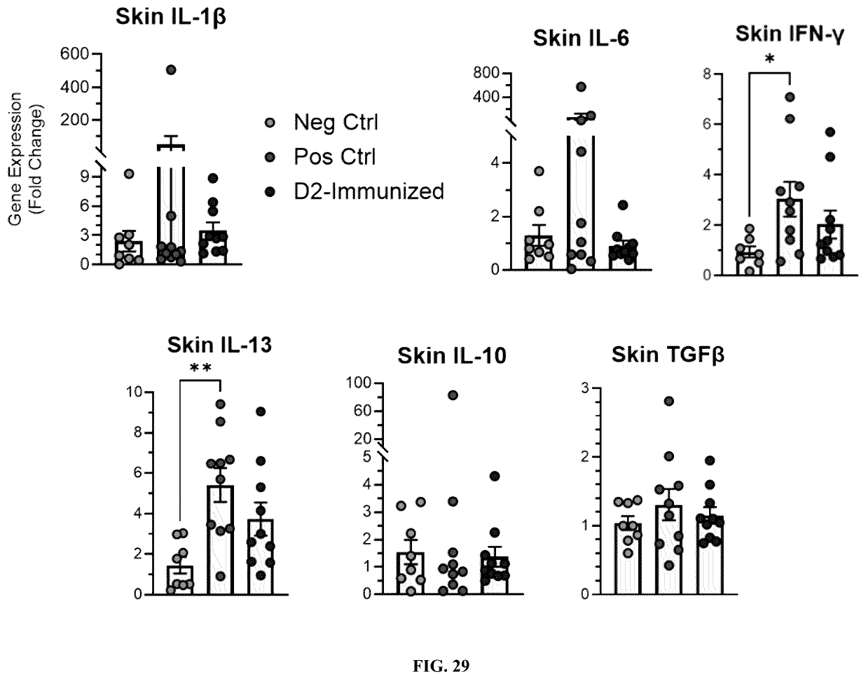

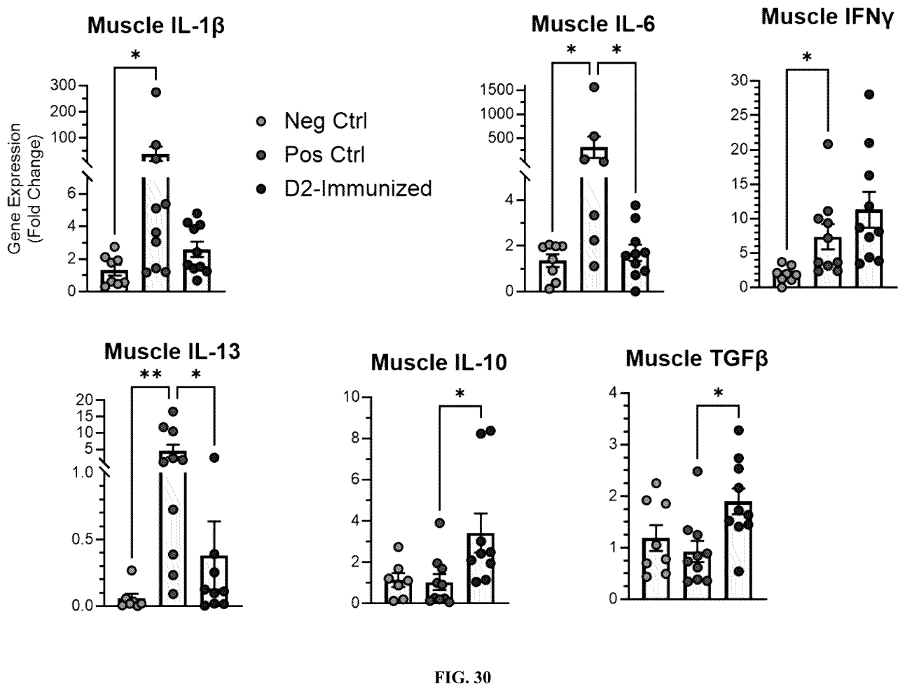

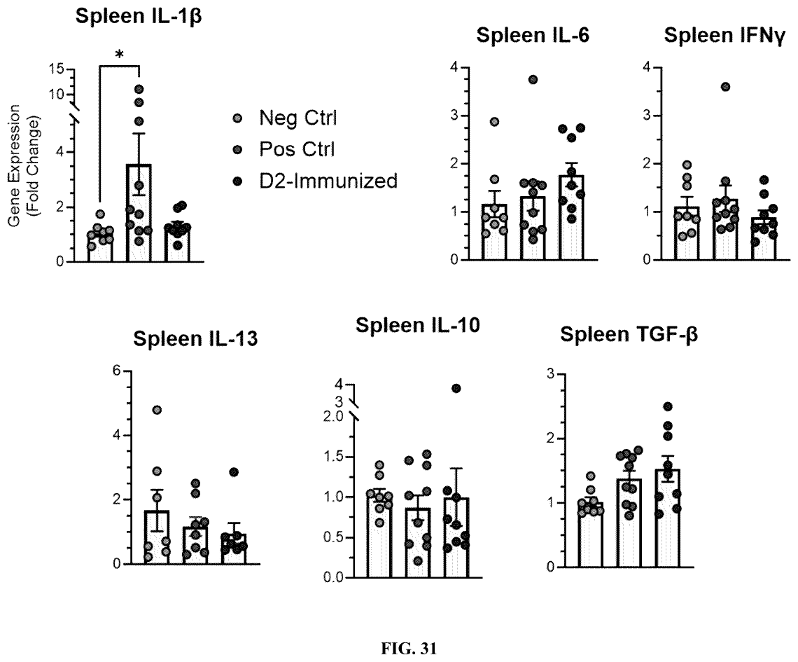

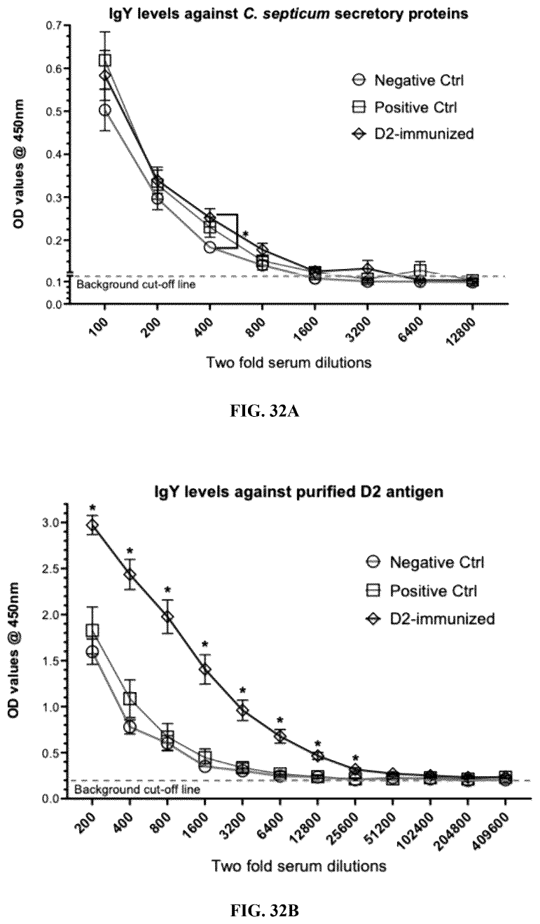

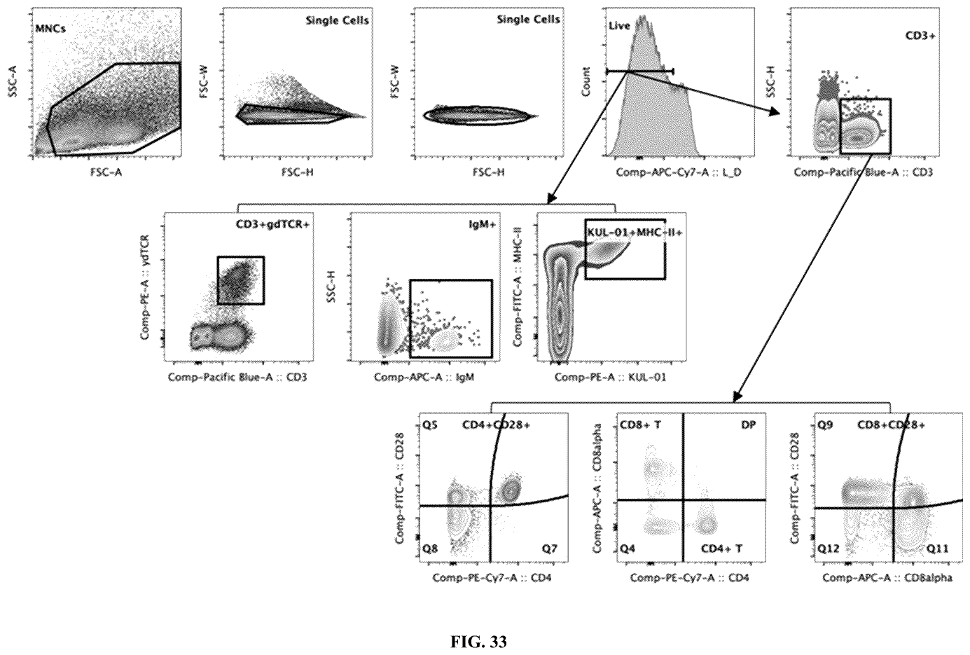

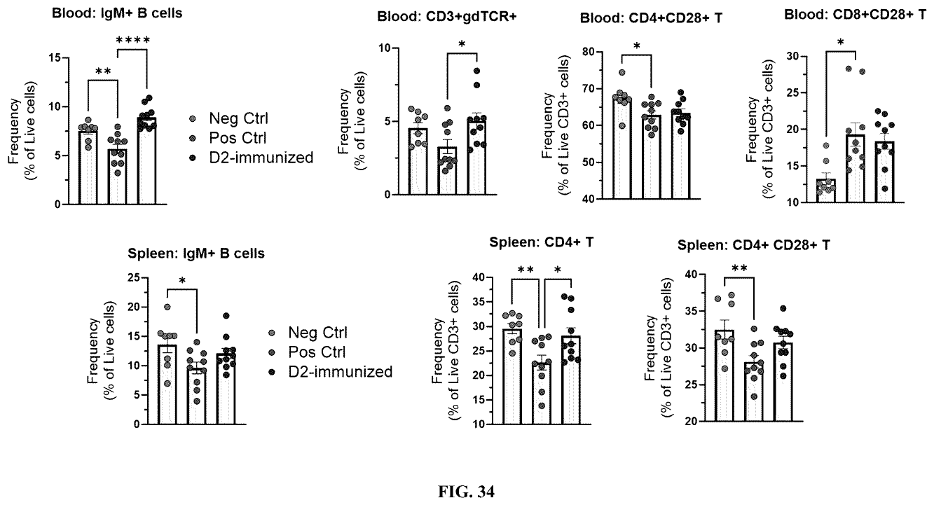

In the skin (FIG. 17), the transcription levels of IL-1β, IL-4, and IL-10 were significantly elevated in the PCx birds compared to the NCx group. Conversely, D2-immunized birds exhibited significantly reduced expression of IL-1β and IL-6 compared to the PCx group. Additionally, IL-1β transcription was significantly downregulated in D1-immunized birds relative to the PCx group. No significant differences in TLR21 gene expression were observed between the groups.

In the muscle (FIG. 18), IL-6 expression was significantly higher in the PCx group compared to the NCx group, while IL-1β expression was significantly downregulated in D2-immunized birds compared to the PCx group. Additionally, IL-4 transcription was significantly downregulated in the D1-immunized group compared to the PCx group. No significant differences were observed in the expression of TLR21 and IL-10 genes between the groups.

In the spleen (FIG. 19), IL-1β and IL-6 transcription levels were significantly higher in the D2-immunized birds compared to the PCx group. No significant differences were observed in the transcription of TLR21, IL-4, and IL-10 genes between the treatment groups.

In the cecal tonsil (CT) (FIG. 20), the transcription levels of TLR21, IL-1β, and IL-10 genes were significantly higher in the PCx group compared to the NCx group. Conversely, the expression of IL-1β, IL-6, and IL-10 genes was significantly downregulated in D2-immunized birds compared to the PCx group. Additionally, TLR21 transcription was significantly lower in D1-immunized birds compared to the PCx group. No significant differences were observed in the expression of the IL-4 gene between the groups.

Immunogenicity: Before the C. septicum challenge, serum samples from immunized turkeys were analyzed to assess levels of IgY antibodies specific to the antigens ntATX-D1 or ntATX-D2. FIG. 21 demonstrates that immunized birds had significantly higher (P<0.05) antigen-specific IgY levels compared to the control group. The rise in the antibody levels was determined through their optical density (OD) values obtained from a series of 9-point serum doubling dilutions.

### Discussion

Clostridial Dermatitis, caused by C. septicum, has posed a serious health and economic threat to the poultry industry, specifically the turkey sector. While therapeutic antibiotics can control CD, their limited availability and declining efficacy coupled with the rising risk of antimicrobial resistance have pushed the research community to find non-antibiotic disease control strategies, such as vaccines. Previously, two non-toxic domains of C. septicum alpha toxin, namely the ntATX-D1 and ntATX-D2, were identified, which were highly antigenic in turkeys by eliciting a protective immunity against CD. However, to facilitate commercial poultry vaccine application, this study aimed to develop an oral recombinant L. lactis probiotic-based vaccines expressing ntATX-D1 and ntATX-D2 proteins. These findings demonstrate that vaccination with the ntATX-D1 or ntATX-D2 vectored L. lactis vaccines were able to reduce CD associated mortality. This was supported by induction of a robust protective immunity against CD based on significantly their mucosal antibody production and limited lesion severity in turkeys.

Lactic acid bacteria, known for their probiotic properties, play a crucial role in enhancing gut health and reducing pathogen burden in the host. Amongst these, L. lactis, a Gram-positive food-grade probiotic bacterium, has been widely used to inhibit the growth of enteric foodborne pathogens due to their properties of producing bacteriocins and antimicrobial peptides. For example, certain bacteriocinogenic strains of L. lactis, namely UL719, UL720 and UL730, can reduce Listeria monocytogenes, Staphylococcus aureus, and C. perfringens infections. Importantly, L. lactis can offer an advanced genetic engineering tool with the development of a versatile and tightly controlled gene expression system based on the autoregulation mechanism of the bacteriocin nisin such as NICE®-vector system. This system has enabled the creation of vectors designed to deliver therapeutic proteins to mucosal tissues. Recently, it was shown that chickens given L. lactis NZ9000 during their early life (1, 7, 14 and 21 days of age) cannot only modulate intestinal immune responses but also can partially reduce C. perfringens-induced Necrotic Enteritis (NE). Several studies have also reported the use of L. lactis host in combination with NICE®-vectored system-based expression of heterologous antigens for reducing the burden of C. difficile in a hamster model and Campylobacter jejuni in chickens, including modulation of host inflammatory responses. In the present study, the L. lactis NZ9000/pNZ8124 host-vector expression system was used to develop recombinant vaccines expressing ntATX-D1 or ntATX-D2 to show that turkeys immunized orally with L. lactis-ntATX-D2 vaccine can be protected against CD.

The C. septicum-induced CD pathogenesis involves severe tissue damage, which is largely attributed to ATX, a cytotoxic necrotizing toxin identified as the key virulence factor. It was recently reported that C. septicum induces a local and systemic inflammatory response during CD in turkeys. These findings indicate that C. septicum field strains exhibit variability in virulence, with disease severity depending on the specific strain. The pathogenesis of the disease is primarily attributed to ATX-mediated tissue and cellular damage. In support of ATX role in CD pathogenesis and immunity, It has been previously shown that antibodies against ATX can play a critical role in preventing CD in turkeys. Previous work by us and others found that commercial turkeys immunized subcutaneously with a C. septicum bacterin-toxoid vaccine developed bacterin-specific serum antibodies and the vaccinated farms had a reduced morbidity and mortality. Lancto et.al., (2014) identified a noncytolytic C. septicum alpha-toxin (NCAT) peptide as a potential vaccine candidate in turkeys and showed that subcutaneous NCAT immunization can provide a partial protection against CD. More recently, through a series of in vitro and in vivo experiments, identified within the C. septicum ATX were the ntATX-D1 and ntATX-D2 regions within the C. septicum ATX that were devoid of toxicity, which were expressed in E. coli as purified recombinant subunit proteins. Subcutaneous immunization of turkeys with the recombinant ntATX-D1 and ntATX-D2 proteins resulted in a significant reduction in mortality, while the birds receiving ntATX-D2 protein vaccine showed a robust protection against CD, as determined by a significant reduction in lesions. In the D2-immunized group, 53.3% of birds (8 out of 15) had lesions with a score of 2 or lower, compared to only 7% (1 out of 13) in the PCx group, indicating that the D2-immunized birds experienced less severe progression of CD. In the present study, a recombinant oral L. lactis vaccines expressing the ntATX-D1 or ntATX-D2 as an oral vaccine against CD in turkeys, was developed. The protective efficacy, following immunization with the L. lactis-ntATX-D2 vaccine, was defined based on complete prevention of mortality and significant reduction in the disease pathology. A case in point was that about only 16% of the L. lactis-ntATX-D2 immunized birds developed severe gross lesions (score of >2), while the unimmunized group (PCx) had 82% of birds with severe gross pathology and ˜30% mortality. Furthermore, the turkeys receiving L. lactis-ntATX-D2 vaccine had significantly reduced histopathology as determined by the inflammatory and gangrenous dermatitis changes in the skin and muscle tissues, suggesting the protective efficacy of ntATX-D2 vaccine antigen when delivered orally. In support of this, previous studies employing subcutaneous delivery of these ntATX antigens had also found that ntATX-D2 was relatively a superior protective antigen than the ntATX-D1. Although the recovery of L. lactis-carrying ntATX-D1 or ntATX-D2 genes from the intestinal content of birds collected at necropsy suggested enteric colonization by the vaccine vector, the differences in protective efficacies observed between the two antigens could be due to variations in their antigen uptake and presentation by immune cells or the immunogenic characteristics. Further work to encapsulate these vaccine vectors into nano- or micro-particles to enhance vaccine delivery and thus, their protective efficacies is required.

The immune gene expression analysis revealed that CD in turkeys involves a marked inflammatory response in the skin, muscle and CT tissues, as noted by an elevated expression of pro-inflammatory cytokine (IL-1β and/or IL-6) genes in the unimmunized+C. septicum-challenged (PCx group) birds. This observation is consistent with previous studies indicating that host inflammatory response against virulent C. septicum seems to contribute to immunopathology, which perhaps may favor the pathogen in disease production. Similarly, studies on gangrenous dermatitis outbreaks in chickens have also shown that host responses included an increased transcription of pro-inflammatory cytokine and chemokine genes in skin tissues, along with augmented frequencies of inflammatory cells in affected birds. These findings collectively suggest that interventions such as vaccines capable of reducing local and/or systemic inflammation could effectively mitigate the progression of CD in turkeys. In the present study, it was found that the L. lactis-ntATX-D2 immunized turkeys had a significant downregulation of IL-1β and/or IL-6 transcription in local (skin and muscle), systemic (spleen), and mucosal lymphoid (CT) tissues, suggesting a positive association between regulation of inflammation and protection against CD. Of note was IL-6, which is a pleotropic cytokine whose dysregulated continual production is known to enhance inflammation and immunopathology. The vaccine-induced dampening of host inflammatory response could be due to a direct effect of probiotic L. lactis colonization or indirectly operating through the induction of pathogen or ATX-neutralizing antibodies. In support of the latter, the immunized birds in the present study were found develop antigen-specific antibodies, while their neutralizing property needs further determination. Another notable observation was that the TLR21 expression in CT tissue was found increased in the unimmunized+challenged group when compared to uninfected control as well as the immunized+challenged groups. It was previously observed that an increased TLR21 transcription in lymphoid tissues of C. perfringens-infected chickens and C. septicum-infected turkeys, suggesting a possible TLR21-mediated pathogen recognition and downstream macrophage-mediated inflammatory response. To this effect, it is reasonable to speculate that vaccine-mediated protective effects resulting in reduced inflammation and the infection load may have caused these TLR21 transcriptional changes in the CT of immunized turkeys in the present study. However, further investigation is needed to elucidate precise mechanisms.

In conclusion, this study demonstrated that L. lactis-based probiotic oral vaccine delivering the ntATX-D2 antigen provides protective immunity against CD in turkeys. The protection mechanisms include induction of antigen-specific IgY antibodies and downregulation of host inflammatory responses.

### Example 3: Immunization of Chickens with Recombinant Clostridium septicum Non-Toxic Alpha Toxin Domain-2 Confers Protective Immunity Against clostridium Dermatitis

Clostridium Dermatitis (CD) in poultry is an economically important disease caused by Clostridium septicum bacteria. In the current era of ‘no-antibiotics-ever’ farming, CD incidences are on the rise, and unfortunately, there are no effective vaccines currently available to prevent CD. Previously, a non-toxic domain #2 of C. septicum alpha-toxin (ntATX-D2) was identified as a vaccine candidate for turkeys that showed protection against CD. Here, the protective efficacy of recombinant ntATX-D2 immunization (via subcutaneous route) of broiler chickens against a C. septicum challenge was evaluated. The results showed that the immunized chickens had significantly higher body weight gain and reduced gross pathology (disease severity) when compared to unimmunized birds, indicating protection against CD. Additional investigations into protective mechanisms showed that the ntATX-D2 immunization led to; 1) A significantly higher levels of antigen-specific serum IgY antibodies, and 2) modulation inflammatory responses in the skin and muscle tissues, as indicated by the significant transcriptional downregulation of proinflammatory cytokine (IL-1β, IL-6, and IFNγ) and upregulation of anti-inflammatory cytokine (IL-10 and TGFβ) genes, when compared to unimmunized control. Furthermore, innate and adaptive cellular mechanisms of protection are currently underway. Collectively, these findings indicate that ntATX-D2 vaccination in broiler chickens can provide protection against CD and the mechanisms of protection seem to operate through anti-ATX antibodies coupled with modulation of local and systemic inflammatory responses.

### Results:

1. Changes in body weight gain and gross pathology (Results in FIGS. 27 and 28).
Findings:
   i) The unimmunized+C. septicum-challenged birds (‘Pos Ctrl’) had significantly reduced body weight gain compared to unimmunized-unchallenged (‘Neg Ctrl’) and ntATX-D2 immunized+C. septicum-challenged (‘D2-immunization’) groups.
   ii) The Pos Ctrl group had significantly higher gross pathology lesions compared to Neg Ctrl and D2-immunization groups.
   iii) No significant changes were observed between the D2-immunization and Neg Ctrl groups.
Conclusion: The ntATX-D2 immunization resulted in significantly reduced pathology with the body weight gains similar to that of Neg Ctrl group birds.
2. Immune gene expression in tissues: Skin (FIG. 29), Muscle (FIG. 30) and Spleen (FIG. 31) tissues.
Findings:
   i) The unimmunized+C. septicum-challenged birds (‘Pos Ctrl’) had significantly higher transcription of Interleukin (IL)-1β in the skin, IL-1β, IL-6 and interferon (IFN)γ in the muscle, and IL-1β genes in the spleen tissues when compared to unimmunized-unchallenged (‘Neg Ctrl’) birds.
   ii) The ntATX-D2 immunized+C. septicum-challenged (‘D2-immunization’) group had significantly higher IL-10 and transforming growth factor (TGF)β in the muscle tissue compared to Pos Ctrl group.
   iii) No significant changes were observed between the D2-immunization and Neg Ctrl groups.
Conclusion: While the unimmunized Pos Ctrl group induced higher inflammation (IL-1β, IL-6 and IFNγ pro-inflammatory cytokine gene expression) in all the tissues compared to Neg Ctrl group, the D2-immunization resulted in significantly reduced IL-6 and increased anti-inflammatory cytokine (IL-10 and TGFβ) gene expression in the muscle compared to Pos Ctrl group.
3. Serum antibody response in immunized chickens. FIG. 32A and FIG. 32B Findings:
   i) Antibody responses were evaluated against two types of proteins; a) C. septicum secreted proteins (which include native alpha-toxin), and b) purified ntATX-D2 protein.
   ii) The D2-immunized birds had significantly higher levels of IgY antibodies compared to Neg Ctrl or Pos Ctrl groups.
Conclusion: The D2-immunization resulted in higher antibody production.
4. Cellular responses in immunized chickens (FIGS. 33 and 34, and Table 5). Findings:
   i) The unimmunized+C. septicum-challenged birds (‘Pos Ctrl’) had significantly lower frequencies of splenic and circulatory CD4+ T cells, CD4+CD28+ T cells and B cells when compared to unimmunized-unchallenged (‘Neg Ctrl’) birds.
   ii) The D2-immunized chickens had significantly higher frequency of splenic CD4+ T cells and circulatory γδ T cells and B cells compared to Pos Ctrl group.
Conclusion: The D2-immunization resulted in increased frequencies of innate γδ T cells, and adaptive CD4+ T cells and B cells when compared to Pos Ctrl group of chickens.

### Overall Conclusions:

1) C. septicum infection induced robust inflammation in both local (skin, FIG. 29 and muscle, FIG. 30) and systemic (spleen, FIG. 31) tissues, as indicated by the increased transcription of pro-inflammatory cytokine (IL-1β, IL-6 and IFNγ) genes.
2) Immunization of broiler chickens using the ntATX-D2 antigen can offer protective immunity against C. septicum-induced CD based on the following clinical parameters.
   a) Reduced gross pathology (FIGS. 27 and 28) and no changes in the body weight gain (FIG. 27).
3) Protection mechanisms seem to include:
   a) Increased expression of anti-inflammatory cytokine (IL-10 and TGFβ) genes in the muscle tissue (FIG. 30).
   b) Increased antibody production (FIG. 32), and induction of higher frequencies of innate and adaptive immune cells.
   c) Table 5 below summarizes the statistically significant findings (FIG. 34) related to cellular responses in the spleen and peripheral blood of D2-immunized and Neg Ctrl groups compared to Pos Ctrl.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
Primers sequences used inConventional
and Real-Time PCR assay.

| | Primer | Annealing | GenBank |
|---|---|---|---|
| Target | Sequence | Temp | Accession |
| Gene | (5′-3′) | (° C.) | # |
| ntATX-D1 | F- | 55 | This work |
| | AAATTCAGTGTGCG | | |
| | GCAGTAGTA | | |
| | (SEQ ID NO: 5) | | |
| | R- | | |
| | CCGTTCCTCAGCAC | | |
| | TCATACCGT | | |
| | (SEQ ID NO: 6) | | |
| ntATX-D2 | F- | 55 | This work |
| | AAATACTGCAACAG | | |
| | TTTCTCCACA | | |
| | (SEQ ID NO: 7) | | |
| | R- | | |
| | CCGTCTAAATCCTG | | |
| | GAACATCTTGTGT | | |
| | (SEQ ID NO: 8) | | |
| IL-1β | F- | 64 | AJ009800 |
| | GTGAGGCTCAACAT | | |
| | TGCGCTGTA | | |
| | (SEQ ID NO: 9) | | |
| | R- | | |
| | GTCCAGGCGGTAGA | | |
| | AGATGAAG | | |
| | (SEQ ID NO: 10) | | |
| IL-6 | F- | 60 | NM_204628.1 |
| | CGTGTGCGAGAACA | | |
| | GCATGGAGA | | |
| | (SEQ ID NO: 11) | | |
| | R- | | |
| | TCAGGCATTTCTCC | | |
| | TCGTCGAAGC | | |
| | (SEQ ID NO: 12) | | |
| IL-10 | F- | 55 | AJ621614 |
| | AGCAGATCAAGGAG | | |
| | ACGTTC | | |
| | (SEQ ID NO: 13) | | |
| | R- | | |
| | ATCAGCAGGTACTC | | |
| | CTCGAT | | |
| | (SEQ ID NO: 14) | | |
| IFNγ | F- | 60 | X99774 |
| | ACACTGACAAGTCA | | |
| | AAGCCGCACA | | |
| | (SEQ ID NO: 15) | | |
| | R- | | |
| | AGTCGTTCATCGGG | | |
| | AGCTTGGC | | |
| | (SEQ ID NO: 16) | | |
| TGFβ | F- | 60 | M31160.1 |
| | CGGCCGACGATGAG | | |
| | TGGCTC | | |
| | (SEQ ID NO: 17) | | |
| | R- | | |
| | CGGGGCCCATCTCA | | |
| | CAGGGA | | |
| | (SEQ ID NO: 18) | | |
| IL-4 | F- | 64 | GU 119892 |
| | TGTGCCCACGCTGT | | |
| | GCTTACA | | |
| | (SEQ ID NO: 19) | | |
| | R- | | |
| | CTTGTGGCAGTGCT | | |
| | GGCTCT | | |
| | (SEQ ID NO: 20) | | |
| IL-13 | F- | 60 | AJ621250 |
| | ACTTGTCCAAGCTG | | |
| | AAGCTGTC | | |
| | (SEQ ID NO: 21) | | |
| | R- | | |
| | TCTTGCAGTCGGTC | | |
| | ATGTTGTC | | |
| | (SEQ ID NO: 22) | | |
| TLR 21 | F- | 60 | NM_001030558.1 |
| | CCTGCGCAAGTGTC | | |
| | CGCTCA | | |
| | (SEQ ID NO: 23) | | |
| | R- | | |
| | GCCCCAGGTCCAGG | | |
| | AAGCAG | | |
| | (SEQ ID NO: 24) | | |
| β-actin | F- | 58 | X00182 |
| | CAACACAGTGCTGT | | |
| | CTGGTGGTA | | |
| | (SEQ ID NO: 25) | | |
| | R- | | |
| | ATCGTACTCCTGCT | | |
| | TGCTGATCC | | |
| | (SEQ ID NO: 26) | | |

TABLE 2
Staining panels used in immunophenotyping.

| | Marker | Conjugate |
|---|---|---|
| Panel 1 | CD4 | PE |
| | CD8 | Pacific Blue |
| | CD28 | FITC |
| | IgM | APC |
| | Live/Dead viable dye | APC-Cy7 |
| Panel 2 | CD4 | Pacific Blue |
| | CD8 | APC |
| | CD44 | FITC |
| | MHC II | PE |
| | Live/Dead viable dye | APC-Cy7 |

TABLE 3
Summary of changes in the expression of immune genes in
treatment groups compared to unchallenged control group.

| | Skin | | | Muscle | | | Cecal tonsil | | | Spleen | | |
|---|---|---|---|---|---|---|---|---|---|---|---|---|
| Genes | PCx | D1 | D2 | PCx | D1 | D2 | PCx | D1 | D2 | PCx | D1 | D2 |
| TLR21 | = | = | + | = | = | = | = | = | = | = | = | = |
| IL-1β | + | = | = | + | + | = | + | = | = | = | = | = |
| IL-6 | = | = | + | + | + | = | = | = | = | = | = | = |
| IFNγ | = | = | = | = | = | = | + | = | = | = | = | = |
| IL-4 | = | = | = | = | = | = | = | = | = | + | = | = |
| IL-10 | = | = | + | = | = | = | = | + | = | = | = | = |

+ denotes increased (P < 0.05) and
− for decreased (P < 0.05),
while = indicates no significant changes in the target gene expression in groups when compared to unchallenged negative control (NCx).
PCx-positive control (unimmunized and challenged),
D1-immunized with ntATX-D1 antigen and challenged and
D2-immunized with ntATX-D1 antigen and challenged.

TABLE 4
Summary of changes in the expression of immune
genes in immunized groups compared to unimmunized group.

| | Skin | | Muscle | | Cecal tonsil | | Spleen | |
|---|---|---|---|---|---|---|---|---|
| Genes | D1 | D2 | D1 | D2 | D1 | D2 | D1 | D2 |
| TLR21 | = | = | = | = | = | = | = | = |
| IL-1β | = | = | = | − | = | − | = | = |
| IL-6 | = | = | = | − | = | − | = | − |
| IFNγ | = | = | = | − | = | = | = | = |
| IL-4 | = | = | = | = | = | = | = | − |
| IL-10 | = | + | = | = | = | = | = | = |

+ denotes increased (P < 0.05) and
− for decreased (P < 0.05),
while = indicates no significant changes in the target gene expression in groups when compared to unimmunized and challenged positive control (PCx).
D1-immunized with ntATX-D1 antigen and challenged and
D2-immunized with ntATX-D1 antigen and challenged.

TABLE 5
Statistical changes when compared to Pos Ctrl group
(unvaccinated and C. septicum challenged)

| | Spleen | | Blood | |
|---|---|---|---|---|
| | | D2- | | D2- |
| Cel-type | Neg Ctrl | immunized | Neg Ctrl | immunized |
| CD4+ T | + | + | NS | NS |
| CD8+ T | NS | NS | NS | NS |
| DP-T | NS | NS | NS | NS |
| CD4+CD28+ T | + | NS | + | NS |
| CD8+CD28+ T | NS | NS | − | NS |
| γδ T | NS | NS | NS | + |
| IgM + B | + | NS | + | + |
| Macrophage | NS | NS | NS | NS |

+ increase (P < 0.05);
− decrease (P < 0.05);
NS—non-significant changes

### REFERENCES

1. ADDIN EN.REFLIST Clark S, Porter R, McComb B, Lipper R, Olson S, Nohner S, et al. Clostridial dermatitis and cellulitis: an emerging disease of turkeys. Avian diseases. 2010;54:788-94.
2. Gornatti-Churria CD, Crispo M, Shivaprasad HL, Uzal FA. Gangrenous dermatitis in chickens and turkeys. J Vet Diagn Invest. 2018;30:188-96.
3. Lancto CA, Foster LK, Kromm MM, McComb B, Williams J, Luke J, et al. A noncytolytic alpha toxin recombinant protein protects turkeys against Clostridium septicum challenge. Avian diseases. 2014;58:566-71.
4. Thachil AJ, McComb B, Andersen MM, Shaw DP, Halvorson DA, Nagaraja KV. Role of Clostridium perfringens and Clostridium septicum in causing turkey cellulitis. Avian diseases. 2010;54:795-801.
5. Clark SRF, L. Current Health and Industry Issues Facing the US Turkey Industry. Proceedings 125th Annual Meeting of the USAHA. 2021.
6. Criollo V, Gaghan C, John F, Orozco E, Thachil A, Crespo R, et al. Immune Response Evaluation in Commercial Turkeys Affected with Clostridial Dermatitis. Avian diseases. 2023;67:1-9.
7. Tellez G, Pumford NR, Morgan MJ, Wolfenden AD, Hargis BM. Evidence for Clostridium septicum as a primary cause of cellulitis in commercial turkeys. J Vet Diagn Invest. 2009;21:374-7.
8. Thachil AJ, Shaw DP, Nagaraja KV. Effects of dexamethasone immunosuppression on turkey clostridial dermatitis. Avian diseases. 2014;58:433-6.
9. Tweten RK. Clostridium perfringens beta toxin and Clostridium septicum alpha toxin: their mechanisms and possible role in pathogenesis. Veterinary microbiology. 2001;82:1-9.
10. Criollo V, John FA, Gaghan C, Fletcher OJ, Thachil A, Crespo R, et al. Characterization of immune responses and immunopathology in turkeys experimentally infected with clostridial dermatitis-producing strains of Clostridium septicum. Veterinary immunology and immunopathology. 2024:110717.
11. Forga A, Robbins K, Smith A, Coles M, Tellez-Isaias G, Vuong CN, et al. Evaluation of Clostridium septicum hemolytic activity, administration route, and dosage volume of a clostridial dermatitis (cellulitis) bacterin-toxoid on humoral immune response in commercial turkeys. Poultry science. 2023; 102:102873.
12. Graham BD, Robbins KM, Teague KD, Graham LE, Merino-Guzman R, Tellez G, et al. Evaluation of the efficacy of a candidate turkey cellulitis/dermatitis oil emulsion vaccine on immune response and mortality under laboratory and commercial conditions. Journal of Applied Poultry Research. 2019;28:818-25.
13. Thachil AJ, McComb B, Kromm M, Nagaraja KV. Vaccination of turkeys with Clostridium septicum bacterin-toxoid: evaluation of protection against clostridial dermatitis. Avian diseases. 2013;57:214-9.
14. John FA, Criollo V, Gaghan C, Armwood A, Holmes J, Thachil AJ, et al. Immunization of turkeys with Clostridium septicum alpha toxin-based recombinant subunit proteins can confer protection against experimental Clostridial dermatitis. PloS one. 2024; 19: e0302555.
15. Mierau I, Kleerebezem M. 10 years of the nisin-controlled gene expression system (NICE) in Lactococcus lactis. Appl Microbiol Biotechnol. 2005;68:705-17.
16. Bermudez-Humaran LG. Lactococcus lactis as a live vector for mucosal delivery of therapeutic proteins. Human vaccines. 2009;5:264-7.
17. Yang XQ, Zhao YG, Chen XQ, Jiang B, Sun DY. The protective effect of recombinant Lactococcus lactis oral vaccine on a Clostridium difficile-infected animal model. BMC Gastroenterol. 2013; 13:117.
18. Kobierecka PA, Olech B, Ksiazek M, Derlatka K, Adamska I, Majewski PM, et al. Cell Wall Anchoring of the Campylobacter Antigens to Lactococcus lactis. Front Microbiol. 2016;7:165.
19. Wang C, Zhou H, Guo F, Yang B, Su X, Lin J, et al. Oral Immunization of Chickens with Lactococcus lactis Expressing cjaA Temporarily Reduces Campylobacter jejuni Colonization. Foodborne pathogens and disease. 2020;17:366-72.

1. A vaccine composition comprising a non-toxic domain of an alpha toxin (ATX) protein and a pharmaceutically acceptable carrier, wherein the non-toxic domain comprises ntATX-D1 or ntATX-D2, or a combination thereof.

2. The vaccine composition of claim 1, wherein the ntATX-D1 comprises at least 80% sequence identity to SEQ ID NO: 2.

3. (canceled)

4. The vaccine composition of claim 1, wherein the ntATX-D2 comprises at least 80% sequence identity to SEQ ID NO: 4.

5. (canceled)

6. The vaccine composition of claim 1, wherein the vaccine composition comprises at least one immunogenic fragment of SEQ ID NO: 2 and SEQ ID NO: 4.

7. The vaccine composition of claim 1, further comprising an additional toxin, or fragments thereof.

8. The vaccine composition of claim 1, further comprising an adjuvant.

9. (canceled)

10. A vector comprising a nucleic acid sequence encoding a non-toxic domain of an alpha toxin (ATX) protein, wherein the non-toxic domain comprises ntATX-D1 or ntATX-D2, or a combination thereof, or an immunogenic fragment thereof.

11. The vector of claim 10, wherein ntATX-D1, ntATX-D2, or a combination thereof are expressed on the same vector or different vectors.

12. The vector of claim 10, wherein the nucleic acid sequence encoding ntATX-D1 comprises at least 80% sequence identity to SEQ ID NO: 1.

13. (canceled)

14. The vector of claim 10, wherein the nucleic acid sequence encoding ntATX-D2 comprises at least 80% sequence identity to SEQ ID NO: 3

15.-18. (canceled)

19. The vector of claim 10, wherein the vector serves as a vaccine.

20. A method of preventing clostridial dermatitis (CD) in a subject, the method comprises administering to the subject a pharmaceutically effective amount of a vaccine composition, wherein the vaccine composition comprises a non-toxic domain of an alpha toxin (ATX) protein and a pharmaceutically acceptable carrier, wherein the non-toxic domain comprises ntATX-D1, ntATX-D2, or a combination thereof.

21. The method of claim 20, wherein the vaccine composition comprises a vector, wherein the vector comprises a nucleic acid sequence encoding a non-toxic domain of an alpha toxin (ATX) protein, wherein the non-toxic domain comprises ntATX-D1, ntATX-D2, or a combination thereof.

22. (canceled)

23. The method of claim 20, wherein the vaccine composition is administered parenterally.

24.-44. (canceled)


SEQUENCE LISTING (26 sequences)

SEQ ID NO: 1 - DNA, 912 bp, Homo sapiens
  misc_feature 1..912: note = Description of sequence: ntATX-D1: Domain 1 gene sequence
  source 1..912: mol_type = genomic DNA
attcagtgtgcggcagtagtaccacatgtacaagcttatgcacttacaaatcttgaagaggggggatatgcaaatcataataatgcttcttcaattaaaatatttggatatgaagacaatgaagatttaaaagctaaaattattcaagatccagagtttataagaaattgggcaaatgtagctcattcattaggatttggatggtgcggtggaacggctaatccaaacgttggacaaggttttgaatttaaaagagaagttggggcaggtggaaaagtatcttatttattatctgctagatacaatccaaatgatccttatgcaagtgggtatcgtgcaaaagatagactttctatgaaaatatcaaatgttagatttgttattgataatgattctataaaattaggtacacctaaagtgaaaaaattagcacctttaaactctgctagttttgatttaataaatgaaagtaaaactgagtctaaattatcaaaaacatttaattatacaacttctaaaacagtttctaaaacagataactttaaatttggagaaaaaataggagtaaaaacatcatttaaagtaggtcttgaagctatagctgacagtaaagttgagacaagctttgaatttaatgcagaacaaggttggtcaaatacaaatagtactactgaaactaaacaagaaagtactacatatactgcaacagtttctccacaaactaaaaagagattattcctagatgtgttaggatcacaaattgatattccttatgaaggaaaaatatatatggaatacgacatagaattaatgggatttttaagatatacaggaaatgctcgtgaagatcatactgaagatagaccaacagttaaacttaaatttggtaaaaacggtatgagtgctgaggaa

SEQ ID NO: 2 - protein, 304 aa, Homo sapiens
  REGION 1..304: note = Description of sequence: ntATX-D1: amino acid sequence
IQCAAVVPHVQAYALTNLEEGGYANHNNASSIKIFGYEDNEDLKAKIIQDPEFIRNWANVAHSLGFGWCGGTANPNVGQGFEFKREVGAGGKVSYLLSARYNPNDPYASGYRAKDRLSMKISNVRFVIDNDSIKLGTPKVKKLAPLNSASFDLINESKTESKLSKTFNYTTSKTVSKTDNFKFGEKIGVKTSFKVGLEAIADSKVETSFEFNAEQGWSNTNSTTETKQESTTYTATVSPQTKKRLFLDVLGSQIDIPYEGKIYMEYDIELMGFLRYTGNAREDHTEDRPTVKLKFGKNGMSAEE

SEQ ID NO: 3 - DNA, 531 bp, Homo sapiens
  misc_feature 1..531: note = Description of sequence: ntATX-D2: Domain 2 gene sequence
  source 1..531: mol_type = genomic DNA
atactgcaacagtttctccacaaactaaaaagagattattcctagatgtgttaggatcacaaattgatattccttatgaaggaaaaatatatatggaatacgacatagaattaatgggatttttaagatatacaggaaatgctcgtgaagatcatactgaagatagaccaacagttaaacttaaatttggtaaaaacggtatgagtgctgaggaacatcttaaagatttatatagtcataagaatattaatggatattcagaatgggattggaaatgggtagatgagaaatttggttatttatttaaaaattcatacgatgctcttactagtagaaaattaggaggaataataaaaggctcatttactaacattaatggaacaaaaatagtaattagagaaggtaaagaaattccacttcctgataagaagagaagaggaaaacgttcagtagattctttagatgctagattacaaaatgaaggtattagaatagaaaatattgaaacacaagatgttccaggatttagac

SEQ ID NO: 4 - protein, 176 aa, Homo sapiens
  REGION 1..176: note = Description of sequence: ntATX-D2: amino acid sequence
TATVSPQTKKRLFLDVLGSQIDIPYEGKIYMEYDIELMGFLRYTGNAREDHTEDRPTVKLKFGKNGMSAEEHLKDLYSHKNINGYSEWDWKWVDEKFGYLFKNSYDALTSRKLGGIIKGSFTNINGTKIVIREGKEIPLPDKKRRGKRSVDSLDARLQNEGIRIENIETQDVPGFR

SEQ ID NO: 5 - DNA, 23 bp, synthetic construct
  misc_feature 1..23: note = Description of sequence: ntATX-D1 Forward primer
  source 1..23: mol_type = other DNA
aaattcagtgtgcggcagtagta

SEQ ID NO: 6 - DNA, 23 bp, synthetic construct
  misc_feature 1..23: note = Description of sequence: ntATX-D1 Reverse primer
  source 1..23: mol_type = other DNA
ccgttcctcagcactcataccgt

SEQ ID NO: 7 - DNA, 24 bp, synthetic construct
  misc_feature 1..24: note = Description of sequence: ntATX-D2 Forward primer
  source 1..24: mol_type = other DNA
aaatactgcaacagtttctccaca

SEQ ID NO: 8 - DNA, 27 bp, synthetic construct
  misc_feature 1..27: note = Description of sequence: ntATX-D2 Reverse primer
  source 1..27: mol_type = other DNA
ccgtctaaatcctggaacatcttgtgt

SEQ ID NO: 9 - DNA, 23 bp, synthetic construct
  misc_feature 1..23: note = Description of sequence: IL-1-beta Forward primer
  source 1..23: mol_type = other DNA
gtgaggctcaacattgcgctgta

SEQ ID NO: 10 - DNA, 22 bp, synthetic construct
  misc_feature 1..22: note = Description of sequence: IL-1-beta Reverse primer
  source 1..22: mol_type = other DNA
gtccaggcggtagaagatgaag

SEQ ID NO: 11 - DNA, 23 bp, synthetic construct
  misc_feature 1..23: note = Description of sequence: IL-6 Forward primer
  source 1..23: mol_type = other DNA
cgtgtgcgagaacagcatggaga

SEQ ID NO: 12 - DNA, 24 bp, synthetic construct
  misc_feature 1..24: note = Description of sequence: IL-6 Reverse primer
  source 1..24: mol_type = other DNA
tcaggcatttctcctcgtcgaagc

SEQ ID NO: 13 - DNA, 20 bp, synthetic construct
  misc_feature 1..20: note = Description of sequence: IL-10 Forward primer
  source 1..20: mol_type = other DNA
agcagatcaaggagacgttc

SEQ ID NO: 14 - DNA, 20 bp, synthetic construct
  misc_feature 1..20: note = Description of sequence: IL-10 Reverse primer
  source 1..20: mol_type = other DNA
atcagcaggtactcctcgat

SEQ ID NO: 15 - DNA, 24 bp, synthetic construct
  misc_feature 1..24: note = Description of sequence: IFN-gamma Forward primer
  source 1..24: mol_type = other DNA
acactgacaagtcaaagccgcaca

SEQ ID NO: 16 - DNA, 22 bp, synthetic construct
  misc_feature 1..22: note = Description of sequence: IFN-gamma Reverse primer
  source 1..22: mol_type = other DNA
agtcgttcatcgggagcttggc

SEQ ID NO: 17 - DNA, 20 bp, synthetic construct
  misc_feature 1..20: note = Description of sequence: TGF-beta Forward primer
  source 1..20: mol_type = other DNA
cggccgacgatgagtggctc

SEQ ID NO: 18 - DNA, 20 bp, synthetic construct
  misc_feature 1..20: note = Description of sequence: TGF-beta Reverse primer
  source 1..20: mol_type = other DNA
cggggcccatctcacaggga

SEQ ID NO: 19 - DNA, 21 bp, synthetic construct
  misc_feature 1..21: note = Description of sequence: IL-4 Forward primer
  source 1..21: mol_type = other DNA
tgtgcccacgctgtgcttaca

SEQ ID NO: 20 - DNA, 20 bp, synthetic construct
  misc_feature 1..20: note = Description of sequence: IL-4 Reverse primer
  source 1..20: mol_type = other DNA
cttgtggcagtgctggctct

SEQ ID NO: 21 - DNA, 22 bp, synthetic construct
  misc_feature 1..22: note = Description of sequence: IL-13 Forward primer
  source 1..22: mol_type = other DNA
acttgtccaagctgaagctgtc

SEQ ID NO: 22 - DNA, 22 bp, synthetic construct
  misc_feature 1..22: note = Description of sequence: IL-13 Reverse primer
  source 1..22: mol_type = other DNA
tcttgcagtcggtcatgttgtc

SEQ ID NO: 23 - DNA, 20 bp, synthetic construct
  misc_feature 1..20: note = Description of sequence: TLR 21 Forward primer
  source 1..20: mol_type = other DNA
cctgcgcaagtgtccgctca

SEQ ID NO: 24 - DNA, 20 bp, synthetic construct
  misc_feature 1..20: note = Description of sequence: TLR 21 Reverse primer
  source 1..20: mol_type = other DNA
gccccaggtccaggaagcag

SEQ ID NO: 25 - DNA, 23 bp, synthetic construct
  misc_feature 1..23: note = Description of sequence: Beta-actin Forward primer
  source 1..23: mol_type = other DNA
caacacagtgctgtctggtggta

SEQ ID NO: 26 - DNA, 23 bp, synthetic construct
  misc_feature 1..23: note = Description of sequence: Beta-actin Reverse primer
  source 1..23: mol_type = other DNA
atcgtactcctgcttgctgatcc